(19) United States
(12) Patent Application Publication
(10) Pub. No.: US 2006/0089335 A1
(43) Pub. Date: Apr. 27, 2006

(54) Compositions and methods for enhancing cognitive function and synaptic plasticity

(76) Inventors: Guosong Liu, Needham, MA (US); Inna Slutsky, Chestnut Hill, MA (US)

Correspondence Address: Monica R. Gerber, M.D., Ph.D.;Choate, Hall & Stewart, Exchange Place, 53 State Street, Boston, MA, 02109, US

(21) Appl. No.: 10/965,451

(22) Filed: Oct. 14, 2004

Related U.S. Application Data
(60) Provisional application No. 60/510,945, filed on Oct. 14, 2003.

(51) Int. Cl. A61K 31/195; A61K 31/66

(52) U.S. Cl. 514/114; 514/561; 514/567

(57) ABSTRACT

The present invention provides compositions and methods for enhancing cognitive function and synaptic plasticity. According to the method, Ca⁺⁺ influx into excitatory neurons (nerve cells) is decreased by treatment with a number of different agents including divalent cations (e.g., Mg⁺⁺), GABA_{B} agonists, GABA_{A} agonists, calcium channel blockers, and/or compounds that decrease action potential firing such as sodium channel blockers. Decreasing Ca⁺⁺ influx results in increased synaptic plasticity and enhanced cognitive function. In particular, decreasing Ca⁺⁺ influx associated with uncorrelated neural activity results in long-lasting increases in synaptic plasticity and cognitive function. This is achieved by administration of agents that cause a voltage-dependent block of NMDA receptors (e.g., divalent cations such as Mg⁺⁺) or by administration of GABA_{B} agonists such as baclofen. The invention further provides screening methods useful in identifying compounds that enhance synaptic plasticity and cognitive function.

### CROSS-REFERENCE TO RELATED APPLICATION

This application claims the benefit of priority to U.S. Provisional Patent Application 60/510,945, filed Oct. 14, 2003, the contents of which are incorporated herein by reference.

### GOVERNMENT SUPPORT

This invention was made with Government Support under Grant No. 1-p30-MH5880-2, awarded by the National Institute of Health. The Government has certain rights in the invention.

### BACKGROUND OF THE INVENTION

The ability to learn and store new information and to recall information over long periods of time is of crucial importance to virtually all aspects of the human experience. Understanding the mechanisms that underlie learning and memory is of immense interest from a scientific, philosophical, and intensely practical point of view. Cognitive impairment associated with memory loss of varying degrees of severity is one of the most common conditions that occurs in the elderly population. Such impairment may include a reduction in the ability to learn new information and/or to retrieve information that has previously been learned. While memory loss has frequently been considered to be an aspect of normal aging, it is also a key feature of Alzheimer's disease (AD), a debilitating condition that affects an estimated 1.9 to 4 million persons in the United States [Clark 2003]. This number is projected to double in the next decade unless a cure or prevention is found [Clark 2003]. Millions more individuals worldwide suffer from the disease, and as average lifespan lengthens it is likely that increases in the prevalence of Alzheimer's disease will occur.

Age-associated decreases in memory have been given a variety of names, including “benign senescent forgetfulness”, “age-associated memory impairment”, “age-associated cognitive decline”, etc. [Petersen 2001; Burns 2002]. These terms are intended to reflect the extremes associated with normal aging rather than a precursor to pathologic forms of memory impairment. For example, age-associated memory impairment has been described as requiring performance at least one standard deviation below the performance of young adults on certain tests indicative of memory function. Attention has recently focused on a condition referred to as “mild cognitive impairment” (or, more specifically, “amnestic mild cognitive impairment”). This term describes individuals with memory impairment more severe than those associated with normal aging but who do not meet the criteria for diagnosis of clinically probable AD. These individuals progress to clinically probable AD at an accelerated rate compared with healthy, age-matched controls [Petersen 2001].

At present there is no accepted therapy for the decline in memory that typically occurs with aging. Current therapies for Alzheimer's disease include acetylcholinesterase inhibitors such as donepezil, rivastagmine, and galantamine. However, these drugs provide only modest benefit in improvement of symptoms, and there is little evidence to suggest efficacy in terms of slowing progression of the disease. In addition, the mechanism by which these drugs produce beneficial effects in Alzheimer's disease remains obscure, since knowledge regarding the role of the cholinergic system in the disease is limited. There is only limited understanding of the relationship between mild cognitive impairment and the later development of Alzheimer's disease. Given the current lack of evidence as to whether treatment for AD can alter the rate or likelihood of progression from mild cognitive impairment to AD, the advisability of instituting such therapy prior to a definitive diagnosis of AD remains unclear [Burns 2002].

It is evident that a considerable need exists in the art for improved methods of enhancing cognitive function, including the prevention and treatment of impairments in learning and memory of all types. In addition, there exists a considerable need for improved understanding of the neurological and neurochemical bases underlying learning and memory, in order to aid rational development of therapies that would enhance normal learning and memory and/or enhance learning and memory in individuals suffering from deficits in these areas. Since learning and memory ultimately depend on synaptic plasticity, there also exists a need in the art for improved methods for studying synaptic plasticity on a cellular and organismic level. Finally, there exists a need in the art for new screening strategies to identify compounds that enhance cognitive function and/or that modulate one or more aspects of synaptic plasticity.

### SUMMARY OF THE INVENTION

The present invention addresses the foregoing needs, among others. The invention provides a fundamentally new understanding of the molecular basis of synaptic plasticity, a phenomenon that is widely considered to be the key mechanism by which memories are encoded and stored in the central nervous system. The inventors have discovered that the major signal that controls synaptic plasticity in a neural network is the background Ca⁺⁺ flux into excitatory neurons in the network. Based on this discovery the invention provides a variety of methods and compositions that enhance cognitive function and synaptic plasticity by decreasing Ca⁺⁺ flux. In particular, it has been discovered that long lasting increases in cognitive function and synaptic plasticity are achieved by treatment with agents that selectively reduce Ca⁺⁺ influx associated with uncorrelated neural activity into excitatory synapses in the network. Such agents include compounds that impose a voltage-dependent block on NMDA receptors (NMDARs), e.g., divalent cations such as Mg⁺⁺. Preferably the block is readily reversible. Preferably the agent is able to impose the block under physiological conditions. Other effective agents alter the release properties of presynaptic terminals. Such agents include GABA_{B} receptor activators, e.g., baclofen.

In one aspect, the invention provides a method for enhancing synaptic plasticity in a neural network comprising the steps of: (i) providing a neural network in which it is desired to enhance synaptic plasticity; and (ii) exposing the neural network to a composition comprising a compound that reduces Ca⁺⁺ flux into excitatory synapses in the neural network. The method may further comprise the step of measuring synaptic plasticity. Synaptic plasticity may be measured before exposing the neural network to the composition, after exposure, or both before and after, e.g., in order to determine the change in synaptic plasticity caused by the compound. In a preferred embodiment, the invention provides a method for enhancing long term synaptic plasticity in a neural network comprising (i) providing a neural network in which it is desired to enhance synaptic plasticity; and (ii) exposing the neural network to a composition comprising a compound that reduces Ca⁺⁺ flux into excitatory synapses in the neural network, wherein the Ca⁺⁺ flux is associated with uncorrelated neural activity.

The invention further provides a method of enhancing cognitive function in a subject comprising steps of: (i) identifying a subject in need of enhancement of cognitive function; and (ii) administering to the subject a composition comprising a compound that selectively reduces Ca⁺⁺ influx associated with uncorrelated neural activity. In certain embodiments of the invention the compound imposes a voltage-dependent block on NMDA receptors. In a preferred embodiment of the invention the compound is a divalent cation such as Mg⁺⁺. In other embodiments of the invention the composition comprises a GABA_{B} receptor activator. In a preferred embodiment the compound is a GABA_{B} receptor agonist such as baclofen. The invention thus provides a method of enhancing cognitive function in a subject comprising steps of: (i) identifying a subject in need of enhancement of cognitive function; and (ii) administering to the subject a composition comprising a compound that imposes a voltage-dependent block on NMDA receptors. The invention further provides a method of enhancing cognitive function in a subject comprising steps of: (i) identifying a subject in need of enhancement of cognitive function; and (ii) administering to the subject a composition comprising a GABA_{B} receptor activator. The subject may be a human being, e.g., a human being suffering from or at risk of a disease or condition such as age-associated memory loss, mild cognitive impairment, or Alzheimer's disease.

The inventors have recognized at least ten pathways through which Ca⁺⁺ flux into excitatory neurons in a neural network may be decreased. Identification of these pathways allows the selection of compounds and compound combinations for enhancing cognitive function and synaptic plasticity. According to certain embodiments the compound is from a class selected from the group consisting of: divalent cations, NMDA receptor inhibitors, AMPA receptor inhibitors, mGluR1 and/or mGluR5 activators, GABA_{B} receptor activators, GABA_{A} receptor activators, muscarinic ACh receptor activators including AChE inhibitors, A1 adenosine receptor activators, voltage-gated Ca⁺⁺ channel inhibitors, and voltage-gated Na⁺ channel inhibitors. Combinations of the foregoing compounds may also be used. The compounds may be administered together or may be administered individually. According to certain embodiments of the invention multiple compounds, which may be from the same class or from different classes, are used. In preferred embodiments, in order to achieve long lasting enhancement of synaptic plasticity and cognitive function, compounds that selectively reduce Ca⁺⁺ influx associated with uncorrelated neural activity are used. Such compounds include agents that impose a voltage-dependent block on NMDA receptors, e.g., divalent cations such as Mg⁺⁺, and agents that increase the activity of GABA_{B} receptors (e.g., GABA_{B} agonists). Other agents mentioned herein can be used for short term enhancement of synaptic plasticity and/or cognitive function.

In another aspect, the invention provides a variety of compositions for enhancing cognitive function and synaptic plasticity. For example, the invention provides a composition comprising at least two compounds, wherein the compounds are members of compound classes selected the group consisting of: divalent cations, NMDA receptor inhibitors, AMPA receptor inhibitors, mGluR1 and/or mGluR5 activators, GABA_{B} receptor activators, GABA_{A} receptor activators, muscarinic ACh receptor activators including AChE inhibitors, A1 adenosine receptor activators, voltage-gated Ca⁺⁺ channel inhibitors, and voltage-gated Na⁺ channel inhibitors, and wherein at least two of the compounds are members of different compound classes.

The invention also provides screening methods that may be used to identify compounds of use for enhancing synaptic plasticity and/or for enhancing cognitive function (e.g., learning and/or memory). The compounds are of use for treating and/or preventing memory impairment. In particular, the invention provides a method of screening a compound comprising steps of: (i) exposing neurons in a cultured neural network to a detectable substance, wherein the substance is taken up by presynaptic terminals that release neurotransmitter; (ii) exposing neurons in the neural network to the compound; (iii) administering a pattern of stimulus to the neurons in the network; (iv) measuring synaptic plasticity; and (v) identifying the substance as an enhancer of cognitive function if the measured synaptic plasticity increases following exposure to the compound. The invention also provides a method of screening a compound comprising steps of: (i) exposing neurons in a cultured neural network to a detectable substance, wherein the substance is taken up by presynaptic terminals that release neurotransmitter; (ii) exposing neurons in the neural network to the compound; (iii) administering a pattern of stimulus to the neurons in the network; (iv) measuring synaptic plasticity; and (v) identifying the substance as an enhancer of synaptic plasticity if the measured synaptic plasticity increases following exposure to the compound. In the afore-mentioned methods, synaptic plasticity can be measured by detecting presynaptic terminals that have taken up the detectable substance and comparing the synaptic strength before and after a stimulus such as theta-burst stimulation.

Where figures either in the Drawing or in the specification or claims depict molecules, or where molecules are referred to in the specification or claims, it is to be understood that the protonation state of various atoms may differ depending on factors such as the pH, as will be understood by one of ordinary skill in the art. All ionized and nonionized forms are included in various embodiments of the invention, and the depiction of a molecule with particular atoms in a charged or uncharged, protonated or unprotonated state is not intended to indicate that the molecules are necessarily in such a state. Furthermore, salts of the compounds are included, as further discussed below.

Unless otherwise stated, structures depicted or named herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted or named herein are also meant to include compounds that differ only in the presence of one or more isotopically enriched atoms. For example, compounds having the present structures except for the replacement of hydrogen by deuterium or tritium, the replacement of a carbon by a ¹³C- or ¹⁴C-enriched carbon, the replacement of nitrogen, phosphorus, or sulfur with an isotope thereof, etc., are within the scope of this invention. Such compounds are useful, for example, as analytical tools or probes in biological assays.

This application refers to various patents and publications. The contents of all of these are incorporated by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Kandel, E., Schwartz, J. H., Jessell, T. M., (eds.), Principles of Neural Science, 4^{th} ed., McGraw Hill, 2000; Cowan, W. M., Südhof, T. C., and Stevens, C. F., (eds.), Synapses, The Johns Hopkins University Press, Baltimore and London, 2001; and Hardman, J., Limbird. E., Gilman, A. (Eds.), Goodman and Gilman's The Pharmacological Basis of Therapeutics, 10^{th} Ed. McGraw Hill, 2001 (referred to herein as Goodman and Gilman). In case of a conflict between the instant specification and one or more of the incorporated references, the specification shall control.

### BRIEF DESCRIPTION OF THE DRAWING

FIG. 1A is a schematic depicting the protocol of stimulation, application of FM 1-43 dye, application of ADVASEP-7, and timing of image acquisition in a hippocampal neuron culture.

FIG. 1B shows fluorescence images of synapses after action potential stimulated loading with the fluorescent dye FM 1-43 (upper panel) and after action potential stimulated exocytosis (lower panel). The bar at the right shows the correlation between color and amount of dye.

FIG. 1C shows a fluorescence images of the same set of synapses loaded with the fluorescent dye AM 1-43 (left), stained for synapsin I (middle), and merged images of the foregoing (right).

FIG. 1D is a bar graph showing the density of synapses that showed activity in response to applied action potentials (AP) compared with the density of synapses identified structurally using staining for synapsin I (structural) and the density of synapses that showed activity in response to stimulation with high K⁺ (functional).

FIG. 2A is a schematic depicting the protocol of theta-burst stimulation, application of FM 1-43 dye, and timing of image acquisition in a hippocampal neuron culture.

FIG. 2B shows fluorescence images of synapses before (left) and after (middle) application of a theta-burst stimulation protocol. The panel at the right shows the same synapses superimposed with a differential interference contrast (DIC) image. The bar at the left shows the correlation between color and amount of dye.

FIG. 2C is a plot comparing the probability of release from individual synapses prior to (F₁) and after (F₂) application of a theta-burst stimulation protocol. Each circle represents an individual synapse.

FIG. 2D is a bar graph plotting (i) the ratio of average fluorescence of individual synapses in a neural network after versus before application of a theta-burst stimulation protocol, where fluorescence indicates release of neurotransmitter (black bar); (ii) the ratio change in the number of synapses at which neurotransmitter is released after versus before application of a theta-burst stimulation protocol (gray bar); (iii) the ratio of synaptic strength in the neural network after versus before application of a theta-burst stimulation protocol (white bar).

FIG. 3A shows fluorescence images of synapses before (left) and after (middle) application of a theta-burst stimulation protocol in a hippocampal neuron culture that was pretreated with TTX, as assessed by uptake of FM 1-43 dye. The left panel shows the same synapses superimposed with a DIC image. The bar at the left shows the correlation between color and amount of dye.

FIG. 3B is a plot showing the number of synapses that released neurotransmitter before and after theta-burst stimulation in a hippocampal neuron culture that was pretreated with TTX, as assessed by uptake of FM 1-43 dye.

FIG. 3C is a plot showing the change in Pr (F₂/F₁) as a function of the initial Pr for individual synapses subjected to a theta-burst stimulation protocol after pretreatment with TTX. Each circle represents an individual synapse.

FIG. 3D is a bar graph showing that the NMDAR antagonist AP-5 inhibits (i) the increase in average release probability for individual synapses (F₂/F₁) in response to stimulation; (ii) the number of synapses that release transmitter in response to stimulation (N₂/N₁); and (iii) the overall strength of the synaptic network (S₂/S₁) in a hippocampal neuron culture that was pretreated with TTX.

FIG. 4A shows the evoked current response over time at a representative postsynaptic location in response to iontophoretic glutamate application in a control culture (0.8 mM Mg⁺⁺).

FIG. 4B shows representative traces of the raw data that was used to construct the plot in FIG. 4A. The peak value of such traces is represented by the circles in FIG. 4A.

FIG. 4C shows the evoked current response over time at a representative postsynaptic location in response to iontophoretic glutamate application in a culture that was pretreated with increased Mg⁺⁺ (1.2 mM) to reduce overall neural activity. The figure shows increased current amplitude over time, indicating insertion of AMPA receptors into the membrane at the postsynaptic location.

FIG. 4D shows that the NDMA receptor antagonist AP5 prevents the increase in postsynaptic current in response to application of glutamate and that washout of the compound reverses this effect. The period during which AP5 was applied is indicated with a black bar at the top of the figure. The data indicates that the increase in postsynaptic current is NDMA receptor dependent, confirming that it is likely to occur via the same mechanism that is thought to underlie LTP.

FIG. 5A and FIG. 5B show fluorescence images of several thousand synapses before (left) and after (right) application of a theta-burst stimulation protocol in a hippocampal neuron culture that was pretreated with baclofen, as assessed by uptake of FM 1-43 dye.

FIG. 5C is a bar graph showing that various treatments, all of which decrease overall activity of a neural network (i) increase the Pr for individual synaptic terminals (F₂/F₁; white rectangles), (ii) increase the number of active presynaptic terminals (N₂/N₁; gray rectangles) and (iii) increase the overall synaptic strength of the network (S₂/S₁; black rectangles). Cultures were treated with the indicated compounds for 2-12 hours.

FIG. 5D is a bar graph showing that various treatments that reduce Ca⁺⁺ flux into postsynaptic locations (i) increase the Pr for individual synaptic terminals (F₂/F₁; white rectangles), (ii) increase the number of active presynaptic terminals (N₂/N₁; gray rectangles) and (iii) increase the overall synaptic strength of the network (S₂/S₁; black rectangles). Cultures were treated with increased Mg⁺⁺ or with Zn⁺⁺ for two weeks or with the Ca⁺⁺ channel blocker nimodipine for 2-12 hours.

FIG. 5E shows effects of AP-5 (20 μM), nimodipine (10 μM), NBQX (1 μM), flunitrazepam (5 μM), and TTX (100 nM) on neuronal activity, expressed as an integral of EPSCs at −60 mV.

FIG. 5F shows that synaptic terminals lose plasticity after prolonged reduction of neural activity with TTX (N=5), NBQX (N=5) and flunitrazepam (N=8).

FIG. 5G shows that chronic incubation for 48 hours with AP-5 (N=6) and nimodipine (N=4) does not trigger an increase of synaptic plasticity.

FIG. 5H shows a comparison of presynaptic strength before (S₁) and after (S₂) TBS in control, 4 and 48 hours TTX-treated cultures. Short-term (4 hours) application of TTX did not significantly change initial presynaptic strength (S₁) (p>0.5, N=5). Long-term (48 hours) incubation induced 1.5 (±0.1)-fold increase in S₁ (p<0.005, N=6), which is significantly lower than the maximal TBS-induced potentiation (S₂=4.9±0.4, N=6). S values were normalized by S₁ of control cultures.

FIGS. 6A-6G show that long-term elevation of [Mg²⁺]ₒ enables synapses to remain highly plastic (A) Representative fluorescent images before (a) and 30 minutes after (b) TBS in Mg²⁺-treated (for 2 weeks) hippocampal culture. (B) Quantification of changes induced by TBS stimulation: (F₂/F₁=2.3±0.1 (gray bar), N₂/N₁=2.1±0.1 (white bar), S₂/S₁=5.5±0.2 (red bar), N=15). (C) Time-course of enhancement of synaptic plasticity induced by 0.4 mM Mg²⁺ (∘), 5 μM AP-5 (●) and 20 μM AP-5 (▪).(D) Mg²⁺-induced enhancement of plasticity is reversible. Elevation of [Mg²⁺]ₒ from 0.8 to 1.2 mM for 48 hours induced the enhancement of synaptic plasticity (the same data as in FIG. 6C) and subsequent reduction of [Mg²⁺]ₒ back to 0.8 mM for 48 hours returned the terminals to a non-plastic state (S₂/S₁=1.1±0.2, N=3, p>0.2). ((E) The peak amplitude of fEPSC before and after LTP induction by TBS. Black circle: recording from neuron in 0.8 mM [Mg²⁺]ₒ cultures; Red circle: in 1.2 mM [Mg²⁺]ₒ cultures. (F) Representative traces of EPSC before and 30 minutes after TBS induction in 0.8 (black traces) and 1.2 (red traces) mM [Mg²⁺]ₒ cultures. (G) Average results of TBS-induced modification of EPSC: fEPSC₂/fEPSC₁ is 1.0±0.1 (N=4) and 2.7±0.6 (N=5), in 0.8 (black bar) and 1.2 (red bar) mM [Mg²⁺]ₒ cultures respectively.

FIG. 6H is a graph showing the dependence on the increase in synaptic strength on extracellular Mg⁺⁺ concentration. The plot shows the ratio of synaptic strength (S) in a neural network at various Mg⁺⁺ concentrations relative to the strength of the network under control conditions (0.8 mM Mg⁺⁺). The graph suggests that there may be an optimum Mg⁺⁺ concentration for inducing synaptic plasticity. Cultures were treated at the indicated concentrations for 2 weeks.

FIG. 6I is a graph showing the dependence on the increase in synaptic strength on extracellular Zn⁺⁺ concentration. The plot shows the ratio of synaptic strength (S) in a neural network at various Zn⁺⁺ concentrations relative to the strength of the network under control conditions (no extra Zn⁺⁺ added to medium). The graph suggests that there may be an optimum Zn⁺⁺ concentration for inducing synaptic plasticity. Cultures were treated at the indicated concentrations for 2 weeks.

FIG. 6J is a graph showing the dependence on the increase in synaptic strength on TTX concentration. The plot shows the ratio of synaptic strength (S) in a neural network at various TTX concentrations relative to the strength of the network under control conditions (no TTX added to medium). The graph suggests that there may be an optimum TTX concentration for inducing synaptic plasticity. Cultures were treated at the indicated concentrations for 2 days.

FIGS. 7A-7D show the effects of baclofen on the EPSC_{NMDA}. FIG. 7A shows EPSC_{NMDA} recorded from neurons in a control culture (FIG. 7A, left) and in a culture treated with baclofen (FIG. 7A, right). The lower tracing in each part of the figure is a recording done in the presense of the selective NR2B blocker ifenprodil. FIG. 7B shows peak eNMDA current recorded from control (open circles) and baclofen-treated (filled circles) neurons in response to a series of stimuli. The left portion of the figure shows currents in the absence of ifenprodil. The right portion of the figure shows currents in the presence of ifenprodil. FIG. 7C is a bar graph showing the percent inhibition of charge transfer by ifenprodil in control and baclofen-treated neurons. The result indicates that baclofen-treated cultures are more sensitive to ifenprodil, indicating that they contain a higher proportion of NMDA receptors containing NR2B subunits. FIG. 7D is a plot showing the percent inhibition of charge transfer by ifenprodil in a control culture (filled circles) and a baclofen-treated culture (open circles) as a function of the decay time constant (τ_{decay}) of the EPSC_{NMDA}.

FIGS. 7E-7L show the long-term effects of elevation of [Mg²⁺]ₒ on the EPSC_{NMDA}. (E) Recordings of evoked EPSCs at −70 mV and +40 mV (in the presence of 50 μM picrotoxin) between paired of neurons in control (0.8), 1.2 and 2 mM Mg²⁺ cultures. (F) The decay time-constant (τ_{decay}) of EPSC_{NMDA} as function of [Mg²⁺]ₒ. (G) N/A ratio (calculated as an integral of G_{NMDA}/G_{AMPA}) at different [Mg²⁺]ₒ. (H) The charge transfer of quantal EPSC_{NMDA} (Q_{NMDA(-70 mV)}) as a function of [Mg ²⁺]. (I and J) Recordings of EPSC_{NMDA} (in the presence of 1 μM NBQX and 50 μM picrotoxin) in control conditions and after application of 3 μM of ifenprodil in neurons from 0.8 and 1.2 mM Mg²⁺ cultures. The peak amplitudes of EPSC during control recordings were normalized to allow for the comparison of drug sensitivity. (K) Ifenprodil has stronger inhibitory effects on EPSC_{NMDA} in neurons from 1.2 mM [Mg²⁺]ₒ cultures (0.8 mM: 45±2%, N=6, p<0.01; 1.2 mM: 80%±4, N=5, p<0.001). (L) 1 μM ifenprodil (IC₅₀) reduced TBS-induced presynaptic potentiation by 48±7% in 1.2 mM Mg²⁺ cultures (N=4, p<0.005).

FIGS. 8A-8F show effects of long term treatment with Mg⁺⁺ in vitro and in vivo. Increase of [Mg] in drinking water increase expression level of NR2B and GLUR1. (A) Addition of 6 mg/kg/day of MgCl₂ to drinking water for 4 months resulted in increase of [Mg] level in CSF (P=0.02) from 1.06±0.03 mM (control, N=8) to 1.19±0.05 (Mg-treated, N=8). (B) Elevation of [Mg] in hippocampal cultures from 1 to 1.2 mM for 1 month resulted in 2.4-fold increase of plasticity of presynaptic terminals (S₂/S₁). (C) Representative Western blotting of NMDAR subunits in control and Mg-treated group. (D) Average results of Western blot analysis of (C) (N=8 for each group). (E) Representative Western blotting of AMPAR subunits and PSD-95 in control and Mg-treated group. (F) Average results of Western blot analysis of (E) (N=8 for each group).

FIGS. 9A-9E are bar graphs showing the effect of magnesium (6 mg/kg/day, added to water) on memory in a novel object recognition task in rats. (A) Percentage of time exploring any of the two identical objects during training. (B) Exploratory preference (percentage of time exploring the novel object) in a short-term memory test carried out 10 min after training. (C) Exploratory preference in a long-term memory test carried out 24 h after training. Only the Mg-treated group showed significant exploratory preference towards the novel object in both short-term (P<0.001) and long-term memory trials (P=0.03). (D) Exploratory preference in a short-term memory test carried out 10 min after double-trial training. (E) Exploratory preference in a long-term memory test carried out 24 hours after double-trial training. Only Mg-treated group showed significant exploratory preference towards the novel object in long-term memory trials (P<0.001). N=9-10 animals per group.

FIGS. 10A-10D show effects of long term baclofen treatment on synaptic plasticity in a neural network and on expression of a memory-associated protein in rats. (A) Pretreatment with baclofen (10 μM) for 6 and 48 hours enhances plasticity of presynaptic terminals (expressed as S₂/S₁) in hippocampal cultures. (B) Baclofen (10 μM, 6 hours treatment) increases the sensitivity of EPSC_{NMDA} to ifenprodil (3 μM) in hippocampal cultures. (C) Treatment with baclofen (0.07 mg/kg/day) increases expression level of NR2B and GLUR1 subunits in rats' hippocampus. Representative Western blotting in control (N=8) and baclofen-treated (N=8) group. (D) Average results of Western blot analysis.

FIGS. 11A-11H show that the voltage-dependent NMDA channel opening is sensitive to physiological variation in [Mg²⁺]ₒ. (A) Exemplary membrane potential trace recorded under current clamp showing uncorrelated (background) and correlated (bursting) patterns of neuronal activity. (B) Normalized peak glutamate-activated NMDA currents plotted against membrane potential in three [Mg²⁺]ₒ (0.8, ∘; 1.2, □; and 2.0 mM, ▪). Membrane potentials were varied from −70 to +50 mV in 20 mV increments and currents were recorded from cultured CA1 pyramidal neurons (N=5). Since the evoked NMDA currents varied among the synapses examined, the amplitudes of NMDA currents were normalized to their maximum values to group all data points together. (C) Normalized g-V relationship, where g/gₘₐₓ was the peak conductance of NMDA channel at 0.8 (∘), 1.2 (□) and 2 mM (∘) [Mg²⁺]ₒ. The continuous lines though points were obtained by fitting g-V relationship with Eqn (2) with the parameters δ and K_{Mg}. (D) The fraction of NMDAR Mg²⁺ block at 1.2 mM [Mg²⁺]ₒ relating to 0.8 [Mg²⁺]ₒ as a function of membrane potential. Note the ˜60% greater attenuation of NMDA current at the sub-threshold range of membrane potential with 1.2 mM [Mg²⁺]ₒ and the decline of this effect when membrane potential crosses the threshold for action potential generation (marked as Vₜ). (E) Dendritic spine visualized with Alexa 633. (F) The time course of NMDA currents and associated Ca²⁺-dependent fluorescence transients (ΔF/F, n=3) in controls and following application of AP-5 (50 μM) from the spine depicted in (E). (G) The amplitude of NMDA currents and Ca²⁺ influx (ΔF/F) at a single spine are linearly correlated (r²=0.99). (F) Elevation of [Mg²⁺]ₒ from 0.8 mM to 1.2 mM led to a parallel reduction of NMDA currents (57±5%) and Ca²⁺ influx (54±7%) (n=3, Vm=−60 mV).

FIG. 12 shows the structure of various GABA_{B} receptor agonists.

FIG. 13 shows the structure of various GABA_{B} receptor positive allosteric modulators.

FIGS. 14A-D illustrate the method used for determination of single vesicle fluorescence, F_{Q}. (A) Experimental protocol used to determine Pr of presynaptic terminals (left). The right panel shows fluorescent images of the same region following loading with 1 AP (a), first unloading (b), loading with 30 AP (c), and second unloading. (B) Decay of average intensity of the fluorescent puncta during unloading using 2 Hz stimulation. Each point represents an average of 841 boutons. (C) Comparison of ΔF in terminals loaded by 1 (gray bars) and 30 APs (red bars) shown in 1A. (D) Histogram of ΔF of terminals stained with 1 APs from one experiment. The red curve represents the best fit to sum of four Gaussian distributions with quantal spacing, F_{Q}=39.8. The first peak represents boutons that did not release vesicles at all; the second peak represents terminals that released one vesicle.

FIG. 15 presents data showing that reduction of neural activity induces enhancement of synaptic plasticity (A) Representative fluorescent images before and 30 minutes after TBS in control (A, B), and TTX-treated (100 nM, 6 hours) (D, E) cultures. DIC images of the same regions (C, F). Fluorescence intensities (arbitrary units) are coded using a pseudocolor transformation shown on the left side of the images. (B) Experimental protocol designed to determine ΔF of synapses before and after plasticity induction. TBS (30 bursts each containing 5 AP@25 Hz, 500 ms inter-burst interval) used as induction protocol. (C) Histograms of ΔF of 704 boutons in control cultures before (solid lines) and after (dotted lines) TBS. There is no change in ΔF distribution in control cultures. (D) Histograms of ΔF 508 boutons in TTX-treated cultures before (solid lines) and after (dotted lines) TBS (D, E). ΔF median increased from 100 to 213, and number of FM detectable boutons increased from 508 to 1608. (E) Plasticity enhancement lasts at least 1.5 hour after TBS induction. (F) Correlation between ΔF of active boutons before and after TBS in control (N=6, open circles) and TTX-treated (N=5, solid circles) cultures. (G) Average magnitude of plasticity (S₂/S₁) for different groups of cultures: 1.1±0.1 for control (N=13), 5.4±0.4 for TTX-treated (N=7), and 1.3±0.2, for TTX-treated in the presence of 50 μM AP-5 during TBS (N=5).

### DETAILED DESCRIPTION OF CERTAIN EMBODIMENTS OF THE INVENTION

The present invention provides a new approach to altering synaptic plasticity within neural networks and to enhancing cognitive function. The inventors have discovered that by decreasing overall activity of excitatory neurons (nerve cells) in a neural network, it is possible to increase the synaptic plasticity of the network. The invention provides a variety of methods for decreasing activity of excitatory neurons in a neural network, and the experiments described herein demonstrate the efficacy of these methods in increasing synaptic plasticity at both the presynaptic and postsynaptic levels. The inventors further discovered that the major signal that links the level of neural activity to the level of synaptic plasticity is Ca⁺⁺ flux into excitatory neurons in the network.

The invention provides a variety of methods and compositions that enhance synaptic plasticity. Without wishing to be bound by any theory, it is believed that the compounds act by decreasing Ca⁺⁺ flux. However, it is to be understood that the compounds may act by one or more alternative mechanisms, in addition to, or instead of, those proposed herein. The scope of the invention is therefore not limited to any particular mechanism of action.

Certain preferred compositions decrease Ca⁺⁺ flux associated with uncorrelated neural activity. Certain preferred compositions comprise a GABA_{B} receptor activator. Other preferred compositions comprise a compound that imposes a voltage-dependent block on NMDA receptors. Treatment with such compositions results in long term enhancement of synaptic plasticity and cognitive function. The compositions and methods of the invention are useful for increasing synaptic plasticity in cultured neural networks, which will facilitate studies of memory and learning, since synaptic plasticity is essential during memory and learning. The compositions and methods are also useful for enhancing cognitive function in vivo, e.g., for the treatment and/or prevention of memory impairment in mammalian subjects such as humans. In particular, the compositions and methods are useful for the treatment and/or prevention of age-associated memory impairment or loss, mild cognitive impairment, and Alzheimer's disease. Certain compositions are useful for short term enhancement of synaptic plasticity and/or cognitive function.

I. Definitions

The term “agonist” is intended to be used as is accepted in the art. In general, the term refers to a substance that can interact with (e.g, bind to) a receptor and initiate a physiological or a pharmacological response characteristic of that induced by interaction of an endogenous ligand with the receptor. Substances generally recognized in the literature as agonists of a particular receptor, e.g., a GABA_{B} receptor, are of use in the methods described herein. The term “agonist” also refers to partial agonists, i.e., compounds that are capable of partially activating a receptor, e.g., activating it to a lesser extent than its endogenous ligand.

The term “antagonist” is intended to be used as is accepted in the art. In general, the term refers to a substance that opposes the receptor-associated responses normally induced by another bioactive agent such as an endogenous ligand. Typically, an antagonist binds to a receptor and prevents binding of an endogenous ligand that would normally activate the receptor, or prevents binding of another agonist to the receptor. The antagonist may or may not induce an effect itself. The activity of a receptor is generally taken to be the activity associated with binding of an endogenous ligand, e.g., GABA, in the case of GABA receptors. Substances generally recognized in the literature as antagonists of a particular receptor, e.g., a GABA_{B} receptor, are of use in the methods described herein

“Approximately” or “about” in reference to a number is generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

“Cognition” generally refers to the process of obtaining, organizing, and using knowledge. Enhancing cognitive function refers to enhancing any aspect of this process, e.g., learning, the performance of mental operations, the storage and/or retrieval of information or thoughts (memory), and/or preventing a decline from a subject's current state. Numerous standardized tests can be used to evaluate cognitive function. Such tests can be used to identify subjects in need of enhancement of cognitive function and/or to monitor the effects of treatment. Suitable tests include, but are not limited to, the Mini-Mental Status Exam (Folstein, 1975), components of the PROSPER neuropsychological test battery (Houx, 2002), etc. Family history, age, and other factors may also be used to identify subjects in need of enhancement of cognitive function.

“Concurrent administration” as used herein with respect to two or more agents, e.g., therapeutic agents, is administration performed using doses and time intervals such that the administered agents are present together within the body, or at a site of action in the body such as in the CNS) over a time interval in less than de minimis quantities. The time interval can be minutes, hours, days, weeks, etc. Accordingly, the agents may, but need not be, administered together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart). According to various embodiments of the invention agents administered within such time intervals may be considered to be administered at substantially the same time. One of ordinary skill in the art will be able to readily determine appropriate doses and time interval between administration of the agents so that they will each be present at more than de minimis levels within the body or, preferably, at effective concentrations within the body. When administered concurrently, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

An “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. A desired biological response may be, for example, (i) an increase in synaptic plasticity; (ii) an improvement in a task requiring cognitive function, e.g., improved performance on a test that measures learning and/or memory; (iii) a slowing in the rate of decline in cognitive function, e.g., as measured by performance on a test that measures learning and/or memory.

“Neural network”, as used herein, refers to a collection of neurons that are interconnected by synapses, such that synaptic transmission (i.e., communication) between neurons in the network may occur. The term may encompass any number of neurons, and some or all of the synapses may appear to be functionally silent. The term may refer either to neurons cultured in vitro or to neurons within the central nervous system of an individual, e.g., a mammal such as a rodent or human being. In general, the invention described herein is of relevance to any neural network. Neural networks comprised of hippocampal neurons, either in vitro or in vivo are primarily discussed herein, but the invention is also of relevance to neural networks comprising cells from elsewhere in the mammalian CNS, e.g., cortical regions, amygdala, etc., either in vitro or in a living subject. In certain embodiments of the invention the neural network comprises or consists of a brain region such as the hippocampus, cortex, amygdala, etc., or the entire brain.

It will be appreciated that every neuron need not form synapses with every other neuron in a neural network. It is sufficient that every neuron forms at least one synapse with at least one other neuron in the network. The neuron may be either the presynaptic or postsynaptic partner. The existence of a synapse may be verified in a number of different ways, including, but not limited to, detecting release of a neurotransmitter in response to a stimulus (which can be performed chemically or by imaging techniques), by measuring the occurrence of an action potential in a first neuron in response to a stimulus to a second neuron, or by histochemical staining, immunohistochemical staining, ultrastuctural imaging, etc.

“Positive receptor modulator” is used to refer to a compound that potentiates the ability of a receptor agonist to activate the receptor. In many instances the compound itself lacks intrinsic activity at the receptor. In some cases the compound itself may have some activity, but typically much less than that of the endogenous agonist. Examples include compounds that act as positive allosteric modulators, inhibitors of agonist metabolism to inactive compounds, inhibitors of agonist transport, etc.

“Sequential administration” of two or more agents refers to administration of two or more agents to a subject such that the agents are not present together in the subject's body at greater than de minimis concentrations. Administration of the agents may, but need not, alternate. Each agent may be administered multiple times.

The term “small molecule”, as used herein, refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Subject”, as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

“Synaptic plasticity” is defined as the ability of a synapse to change its strength in response to a pattern of stimulation (i.e., one or more electrical or chemical stimuli), wherein the alteration in strength typically outlasts the event that triggers it. A synapse that exhibits this property is said to be plastic, or to display synaptic plasticity. A neural network in which some or all of the synapses exhibit plasticity is also said to exhibit synaptic plasticity. Synaptic plasticity may be considered to exist at the level of the presynaptic terminal, the postsynaptic terminal, or both. Thus a synapse is said to exhibit presynaptic plasticity if presynaptic strength is altered in response to a pattern of stimulation. A synapse is said to exhibit postsynaptic plasticity if postsynaptic strength is altered in response to a pattern of stimulation, and/or if the probability that an action potential will be generated in response to a second pattern of stimulation is altered as a result of a first pattern of stimulation.

“Synaptic strength” of a given synapse may be assessed by measuring one or more indicators of presynaptic strength, postsynaptic strength, or both. In general, presynaptic strength refers to properties including (i) the amount of neurotransmitter released in response to a pattern of stimulation; and/or (ii) the probability of neurotransmitter release in response to a pattern of stimulation. The product of (i) and (ii) provides an overall measure of presynaptic strength. Postsynaptic strength refers to properties including (i) the size of the postsynaptic current or potential induced by a fixed amount of neurotransmitter or other stimulus, e.g., an electrical stimulus; and/or (ii) the probability of firing of an AP for a fixed amount of input. Overall synaptic strength reflects a combination of presynaptic and postsynaptic strength. Overall synaptic strength may be determined by combining measures of presynaptic and postsynaptic strength (e.g., by adding, multiplying, etc.). Alternatively, overall synaptic strength may be measured directly, e.g., by stimulating individual presynaptic neuron(s) and recording the evoked response at the corresponding postsynaptic neuron(s). For purposes of the present invention, a synapse will be said to increase its synaptic strength if it increases its presynaptic strength or its postsynaptic strength, or both. A synapse will be said to decrease its synaptic strength if it decreases its presynaptic strength or its postsynaptic strength, or both. One of ordinary skill in the art will recognize that other parameters indicative of synaptic strength may be used, and parameters may be combined in various ways to arrive at a measurement of synaptic strength. One of ordinary skill in the art will also appreciate that a variety of measurement techniques may be applied to assess parameters associated with synaptic strength.

“Treating”, when used with respect to a desired therapeutic effect in a subject such as a human being, can include reversing, alleviating, inhibiting the progress of, preventing, or reducing the likelihood of the disease, disorder, or condition to which such term applies, or one or more symptoms or manifestations of such disease, disorder or condition. “Preventing” refers to causing a disease, disorder, condition, or symptom or manifestation of such, or worsening of the severity of such, not to occur.

“Uncorrelated activity” refers to spontaneous firing of neurons that occurs on a stochastic basis and lacks a spatial or temporal relationship to the firing of other neurons in the same network. Correlated activity refers to firing that has a spatial and/or temporal relationship to firing of other neurons in the same network and is typically associated with a stimulatory input such as a sensory input or other physiologically meaningful input.

III. Synapses, Synaptic Transmission, and Neurotransmitter Systems

In order to facilitate an understanding of the invention this section provides a discussion of synapses, the process of synaptic transmission, and neurotransmitter systems, focusing on aspects of relevance to the present invention. Further details are to be found in Kandel, supra, and Cowan, supra.

Synapses are “specialized intercellular junctions between neurons or between neurons and other excitable cells where signals are propagated from one cell to another with high spatial precision and speed.” [De Camilli, in Cowan, supra]. They are the primary sites of intercellular communication in the mammalian nervous system. Synapses may be classified as electrical or chemical, based on the mechanism by which transmission takes place. Chemical synapses are of primary interest with respect to the present invention, and the term “synapse” as used herein will be assumed to refer to a chemical synapse.

In general, the basic structure of a synapse consists of a close juxtaposition of specialized regions of the plasma membrane of two neurons, referred to as the presynaptic and postsynaptic neurons, to form a synaptic junction. Synapses may also exist between different specialized regions of the plasma membrane of a single neuron, in which case the regions may be referred to as the presynaptic location and postsynaptic locations. These terms may also be applied when the synapse involves two neurons. The region between the neurons or locations is referred to as the synaptic cleft. On the presynaptic side of the junction a cluster of vesicles containing chemical substance(s) (neurotransmitters) is closely associated with the cell membrane, while on the postsynaptic side there are receptors specialized to respond to binding of the neurotransmitter(s) under appropriate conditions.

It will be appreciated that a neuron is typically either a presynaptic or postsynaptic neuron at any particular synapse, but that any given neuron may participate in multiple synapses and may be pre- or postsynaptic at different synapses. Furthermore, multiple synapses may exist between any given pair of neurons. In addition, retrograde signaling can occur in which stimulation by the presynaptic neuron causes release of molecules by the postsynaptic neuron at the same synapse, which then diffuses back across the synaptic cleft to act on the presynaptic cell. It is noted that although for purposes of description it is frequently assumed herein that the presynaptic and postsynaptic portions of the synapse are present on two different cells, a synaptic junction can also exist between different portions of a single cell, as mentioned above. For example, a synapse may be formed at a junction between an axon (presynaptic) and a dendrite (postsynaptic) of a single cell. Such a configuration is referred to as a recurrent connection.

Normally, synaptic transmission occurs when an electrical signal (e.g., an action potential), in the presynaptic neuron triggers vesicle fusion with the presynaptic plasma membrane, causing release of neurotransmitter, which then diffuses across the synaptic cleft between the presynaptic and postsynaptic neurons. Synaptic transmission may also be artificially stimulated in a number of ways, e.g., by application of an electrical stimulus or by application of a compound that causes opening of ion channels in the presynaptic membrane. In general, the signal that causes transmitter release results in opening of voltage-dependent Ca⁺⁺ channels on the presynaptic neuron plasma membrane, or to release of Ca⁺⁺ from intracellular stores. Ca⁺⁺ acts as a trigger for vesicle fusion.

Binding of the neurotransmitter to a postsynaptic receptor may have any of a variety of effects, depending upon the particular neurotransmitter and receptor, as discussed further below. However, in general, the eventual result of neurotransmitter binding is to alter the electrical potential across the postsynaptic membrane, i.e., to cause depolarization or hyperpolarization of the postsynaptic neuron. Depolarization leads to the opening of voltage-dependent ion channels (e.g., Na⁺, K⁺, and/or Ca⁺⁺ channels). Entry of Na⁺ and/or Ca⁺⁺ leads to further depolarization, while subsequent efflux of K⁺ repolarizes the neuron. If sufficient depolarization occurs, then an electrical signal (action potential) is propagated in the postsynaptic neuron. Neural activity of a neuron may be defined as the average rate of firing of an action potential by that neuron. Neural activity of a neural network may be defined as the average rate of firing of action potentials by neurons within the network.

In general, an isolated stimulus that results in hyperpolarization is said to be inhibitory since it reduces the likelihood that the postsynaptic neuron will fire an action potential, while a stimulus that results in depolarization is said to be excitatory since it increases the likelihood that the postsynaptic neuron will fire an action potential. More complicated effects can result when multiple stimuli occur in relatively quick succession, some of which are discussed below.

Presynaptic neurons can be classified on the basis of whether their effect on a postsynaptic neuron is inhibitory or excitatory. Thus if neurotransmitter release by a presynaptic cell tends to inhibit or reduce the likelihood of subsequent firing of an action potential by the postsynaptic cell, the presynaptic cell is considered to be an inhibitory neuron with respect to that synapse, and the released neurotransmitter is considered to be an inhibitory neurotransmitter with respect to that synapse. If neurotransmitter release by a presynaptic cell tends to stimulate or increase the likelihood of subsequent firing of an action potential by the postsynaptic cell, the presynaptic cell is considered to be an excitatory neuron with respect to that synapse, and the released neurotransmitter is considered to be an excitatory neurotransmitter with respect to that synapse. In general, most nerve cells in the mammalian CNS receive inputs from many neurons, which typically include both inhibitory and excitatory neurons. These inputs are integrated to produce a single response. Thus, for example, if a cell receives both inhibitory and excitatory inputs, the “decision” of whether to fire an action potential typically depends on the overall balance between inhibition and excitation.

A variety of neurotransmitters exist, and each of these transmitters can interact with a variety of different receptors. Whether a given transmitter acts in an excitatory or inhibitory fashion depends on the nature of the receptor to which it binds on the postsynaptic cell. However, in the mammalian CNS most transmitters act primarily on receptors that are either excitatory or inhibitory. The primary excitatory neurotransmitter of concern in the context of the present invention is glutamate, while the primary inhibitory neurotransmitter of concern herein is gamma-aminobutyric acid (GABA). Glutamate can also act as an inhibitory transmitter when it binds to certain receptors.

Neurotransmitters act either directly or indirectly to control the opening of ion channels in the postsynaptic cell. Neurotransmitter receptors may be divided into two categories based on whether they gate (i.e., open or close) ion channels directly or whether they act by other mechanisms such as by altering intracellular reactions such as phosphorylation, which then results in gating of ion channels. Neurotransmitters in the former category are called ionotropic receptors while those in the latter category are referred to as metabotropic receptors. In the case of ionotropic receptors, binding of the neurotransmitter causes a conformational change in the receptor that leads to opening of the channel. In the case of metabotropic receptors, binding of the neurotransmitter triggers intracellular events such as the production of second messenger molecules, which ultimately leads to ion channel opening.

Both ionotropic and metabotropic glutamate receptors are found in the mammalian CNS. Ionotropic glutamate receptors may be divided into three major subtypes: NMDA, AMPA, and kainate. AMPA receptors act as ion channels that conduct both Na⁺ and K⁺ upon binding of glutamate (or another agonist). NMDA receptors differ from other glutamate receptors in a number of significant ways. First, the NMDA receptor controls a channel that conducts Ca⁺⁺ in addition to Na⁺ and K⁺. In addition, opening of the channel depends on membrane voltage as well as on the binding of the transmitter (or another agonist). The voltage dependence arises due to the presence of a Mg⁺⁺ ion, which binds to a site within the channel and acts as a plug to block the channel. At resting membrane potential Mg⁺⁺ is tightly bound, but when the membrane is depolarized (e.g., by entry of Na⁺ through AMPA receptors or by entry of Ca⁺⁺ or Na⁺⁺ through voltage-gated Ca⁺⁺ or Na⁺ channels), Mg⁺⁺ is expelled, allowing Na⁺ and Ca⁺⁺ to enter. Mg⁺⁺ therefore imposes a voltage-dependent block on NMDA receptors. Thus, in general, maximum current flow through NMDA receptors requires both the presence of glutamate and a certain level of depolarization due to other excitatory inputs. In accordance with the invention, Mg⁺⁺ acts to effectively block Ca⁺⁺ influx associated with uncorrelated activity, since this typically occurs when the membrane is hyperpolarized, but does not typically block Ca⁺⁺ influx associated with correlated activity, e.g., activity that occurs as a result of stimulation, since excitatory inputs under these conditions depolarize the membrane sufficiently to relieve the block.

Entry of Ca⁺⁺ leads to a variety of long-lasting biochemical modifications in the synapse and elsewhere in the cell, thus providing a potential basis for long-term storage of information. Since maximal functioning of NMDA receptors requires depolarization of the postsynaptic membrane, which occurs as a result of presynaptic activity, the functioning of NMDA receptors provides a mechanism for modifying synaptic strength based on activity, i.e., a mechanism for achieving synaptic plasticity. It should also be mentioned that the NMDA receptor comprises two subunits, referred to as NR1 and NR2, of which at least two NR2 subtypes, NR2A and NR2B exist. The significance of these subunits is discussed further below.

Metabotropic glutamate receptors indirectly gate ion channels through activation of second messenger systems. A number of metabotropic glutamate receptors exist. Of primary interest herein are those known as mGluR1 and mGluR5. At excitatory synapses of concern herein, these receptors are found primarily postsynaptically.

GABA receptors may be divided into three classes: GABA_{A}, GABA_{B}, and GABA_{C}. All three classes are inhibitory, but their distribution within the CNS differs. In addition, they are activated and/or inhibited by different classes of compounds. GABA_{B} receptors present on the presynaptic side at excitatory synapses and GABA_{A} receptors present on the postsynaptic side at inhibitory synapses are of particular relevance for purposes of the present invention. In general, GABA_{A} receptors are ionotropic receptors that exert their inhibitory effects by increasing conductance of chloride ions (Cl⁻), while GABA_{B} receptors are metabotropic and exert their inhibitory effects via coupling to G proteins. GABA_{B} receptors are thought to act on presynaptic terminals by decreasing Ca⁺⁺ flux and to exert their inhibitory effects on postsynaptic terminals in part by increasing conductance of potassium ions (K⁺). Like GABA_{A} receptors, GABA_{C} receptors also exert an inhibitory effect by increasing conductance of chloride ions.

GABA_{B} receptors are believed to exist largely as heterodimers, consisting of GABA_{B(1)} and GABA_{B(2)} subunits, both of which are typically required for formation of a functional receptor. See Bowery, 2002; Vacher, C., 2003; and Bettler, B., 2004, for comprehensive reviews on GABA_{B} receptors and their properties and discussion of compounds that activate or inhibit GABA_{B} receptor activity, and see references cited in each of the foregoing for additional details. The GABA_{B(L)} subunit was cloned in 1997 (Kaupmann, 1997), and the GABA_{B(2)} subunit shortly thereafter (Jones, 1998; Kaupmann, 1998; Ng, 1999). Various isoforms, including GABA_{B(1)} isoforms known as GABA_{B(1a)} and GABA_{B(1b)} are known to exist. For purposes of the present invention, all GABA_{B}-binding receptors, whether composed of GABA_{B(1)}, GABA_{B(2)}, or isoform(s) of either, are considered GABA_{B} receptors.

II. Synaptic Plasticity and Information Storage

Considerable evidence suggests that synaptic plasticity is essential for achieving proper organization of neural circuits during early development, and for the storage of information in functional neural networks. Indeed altering the strength of connections between neurons (synaptic strength) is commonly believed to be a mechanism by which memory traces are encoded and stored in the CNS [Martin 2000]. It is thus widely assumed that activity-dependent synaptic plasticity is induced at appropriate synapses during memory formation and plays a key role for the information storage underlying the type of memory mediated by the brain area in which that plasticity is observed [Martin 2000]. Whether such activity-dependent synaptic plasticity is both necessary and sufficient for information storage remains a subject of debate, but its overall importance seems clear.

One widely studied example of synaptic plasticity thought to be of major importance in learning and memory is known as long-term potentiation (LTP). In general, LTP refers to a property of certain neural networks in which a brief, high frequency stimulus pattern increases the amplitude of subsequent excitatory postsynaptic potentials in the target neurons. LTP is known to exist in a variety of pathways in the CNS, including three major pathways in the hippocampus. In fact it has been observed in a wide variety of glutamatergic synapses in the CNS. LTP can be induced in laboratory studies in cultured neural networks or intact brain slices by application of a high frequency stimulus or by directly depolarizing postsynaptic cells while maintaining low frequency stimulation. One widely applied technique for inducing LTP is referred to as theta-burst stimulation, which mimics stimulation patterns known to occur in the hippocampus.

Despite years of extensive research, the regulation of synaptic plasticity under physiological conditions has not heretofore been explained. Studies of the developmental regulation of synaptic plasticity in visual and somatosensory cortex provide important clues as to the endogenous signals that regulate the magnitude of synaptic plasticity. Pioneering works by Hubel and Wiesel [Hubel & Weisel 1970] provide the first important insight as to how synaptic plasticity is regulated during early development. Deprivation of the visual inputs from one eye can dramatically alter synaptic projections in favor of the other. This activity-dependent modification of synaptic connections occurs during early phases of development (critical period). Since then, many other experiments have been carried out in the hope of determining the mechanisms of this activity-dependent synaptic modification [Katz & Schatz 1996]. Essentially, it has been found that synapses in the visual system are plastic during early development. The neural activity initiated by visual inputs after eye opening triggers synaptic modifications, resulting in the fine-tuning of synaptic connections [Katz & Schatz 1996]. At the same time, neural activity from visual inputs also provides the signal terminating the plasticity of synapses in the visual pathways, as is evidenced in the decline of synaptic plasticity after eye opening. Dark rearing can prevent the down-regulation of synaptic plasticity.

The inventors have recognized that the overall level of activity in a neural network is an important parameter in regulating synaptic plasticity of the network and that it can be modulated to increase synaptic plasticity and cognitive function by administration of appropriate compounds.

IV. Molecular Mechanisms to Enhance Synaptic Plasticity

A. Experimental System and Approach

The inventors sought to determine the molecular and cellular mechanisms that couple the level of neural activity to the plasticity of synapses. In particular, the inventors sought to (i) identify the general conditions necessary for a synapse to be plastic and to determine whether these conditions indeed include the level of prior activity and (ii) determine what physiological and molecular properties of the pre- and postsynaptic locations are regulated by these conditions and are ultimately responsible for conferring plasticity to these terminals.

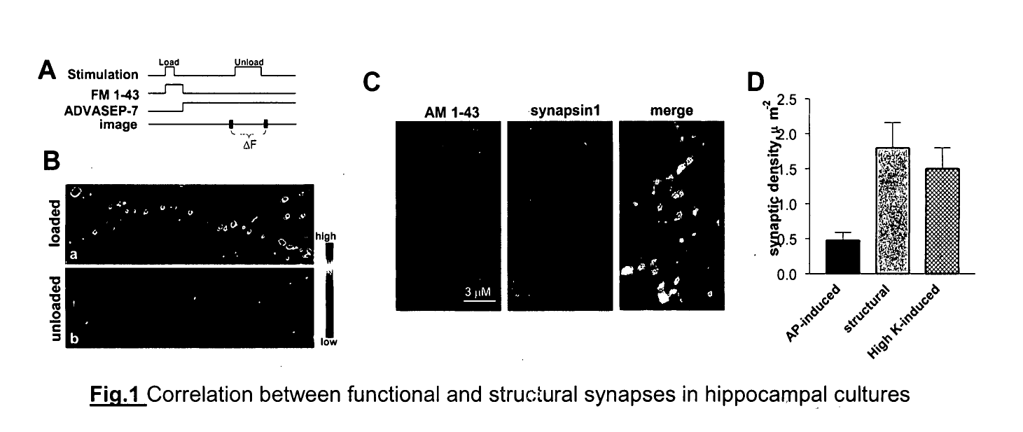

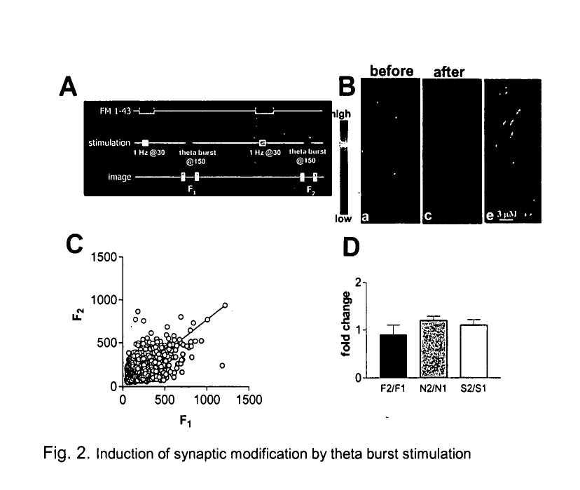

As described in Example 1, the inventors used activity-dependent dye uptake as an index to study the probability of neurotransmitter release (Pr) at single presynaptic terminals within neural networks. Strikingly, the inventors discovered that a large proportion of synaptic terminals has extremely low Pr (<0.04) and is functionally silent. FIG. 1 shows the correlation between synapses identified structurally and functionally and indicates the presence of many synapses that are identifiable structurally but that are not active. The presence of silent synaptic terminals within neural networks provides a great potential to rapidly up-regulate the strength of their synaptic connections. In order to determine whether these silent synapses can be converted to functional ones by manipulating patterns of stimulation, the inventors applied theta-burst stimulation to the network. As shown in FIG. 2, this stimulus was not sufficient to alter their Pr and convert these silent presynaptic terminals to functional ones. This system therefore provided an appropriate context in which to identify methods of enhancing synaptic plasticity and to determine whether manipulating the overall level of activity in a neural network would increase plasticity. To this end, the inventors applied a variety of treatments to neural networks. These experiments revealed that decreasing overall activity of a neural network by any of a variety of methods enhances synaptic plasticity. In particular, the inventors identified overall Ca⁺⁺ flux into excitatory synaptic locations and neurons as a major regulator of synaptic plasticity. The inventors further discovered that treatments that reduce overall Ca⁺⁺ flux into excitatory synapses are effective in enhancing synaptic plasticity and cognitive function. In particular, it was discovered that long term enhancements in synaptic plasticity and cognitive function can be achieved by selectively reducing the component of Ca⁺⁺ flux into excitatory synapses that is associated with uncorrelated neural activity. Long term enhancement refers to enhancement that persists for a period of at least 48 hours, preferably longer than 1 week, longer than 2 weeks, longer than 1 month, longer than 3 months, longer than 6 months, longer than 1 year, or indefinitely. Preferred agents include compounds that impose a voltage-dependent block on NMDA receptors and agents that increase GABA_{B} receptor activity. Treatment may be continued throughout the period during which enhancement occurs.

The compounds and methods are useful for enhancing synaptic plasticity and cognitive function in a subject or for enhancing synaptic plasticity in a cultured neural network. The methods of the invention may be applied to enhance any aspect of cognitive function, e.g., learning and/or memory, within a subject. The subject may be a patient at risk of or suffering from a condition or disorder associated with memory impairment, such as those mentioned above. The compounds may be administered during all or part of the period during which enhancement is desired. Preferably the compounds are administered at intervals during the time over which enhancement persists. For example, the compounds can be administered 34 times daily, 1-2 times daily, every other day, weekly, etc. The inventors have further discovered preferred dosage ranges and/or concentrations within the body for these compounds. It may be preferred to maintain an effective concentration within the body over a time period during which cognitive enhancement is desired. Since, in general, it is desirable to maintain cognitive function throughout life, the compounds may be administered indefinitely.

The invention further provides a method for enhancing synaptic plasticity in a neural network comprising the step of exposing the neural network to a treatment that reduces overall activity of the network. Preferably the treatment reduces overall activity of excitatory synapses within the network. The invention further provides a method of enhancing synaptic plasticity in a neural network comprising the step of exposing the neural network to a treatment that decreases overall Ca⁺⁺ flux into neurons within the network. Preferably the treatment reduces overall Ca⁺⁺ flux into excitatory neurons within the network. In certain embodiments of the invention the treatment comprises administration of a chemical compound or combination of chemical compounds to the network. The composition may comprise a single compound or multiple compounds. In certain preferred embodiments of the invention the neural network comprises hippocampal neurons. Preferably the neural network comprises mammalian neurons.

The neural network may be present in vitro, e.g., in cultures of dissociated neurons or in a tissue slice, or in vivo, i.e., within an intact organism. The sections below describe the inventive compositions and methods for enhancing cognitive function and synaptic plasticity in further detail.

It is noted that when reference is made to the action of certain compounds to stimulate or inhibit a receptor or channel, such stimulation or inhibition may be direct or indirect. In general, a compound is considered to stimulate a receptor or channel if the effect of exposure to the compound under any given set of conditions (e.g., in vitro or in vivo) is to increase the activity of the receptor or channel, by any means. A compound is considered to inhibit a receptor or channel if the effect of exposure to the compound under any given set of conditions (e.g., in vitro or in vivo) is to decrease the activity of the receptor or channel, by any means. It is also noted that a variety of synonyms may be used to describe the effect of a compound on a receptor or channel. For example, a compound may be said to “stimulate”, “activate”, “enhance”, etc., the receptor or channel or its activity to indicate compounds or treatments whose effect is to increase the activity of the receptor or channel. Such terms are generally used interchangeably herein. Similarly, the terms “inhibit”, “repress”, etc., are generally used interchangeably herein to indicate compounds or treatments whose effect is to decrease the activity of the receptor or channel. Thus agonists are generally substances that activate a receptor while antagonists are generally substances that inhibit a receptor. An activity of a receptor is generally taken to be an activity associated with binding of an endogenous ligand, e.g., GABA, in the case of GABA receptors. For example, activation may result in increased flux of an ion across a cell membrane, inhibition or activation of a protein such as a G protein, etc.

Some compounds may activate or inhibit a receptor by modulating the effect of a second compound (e.g., an endogenous compound) on the activity of the receptor or channel. Certain compounds that activate by such a mechanism are referred to as positive allosteric modulators. These compounds typically bind to a receptor at a site distinct from that to which the endogenous ligand binds, and their binding potentiates the effect of the endogenous ligand. Thus the effect of an allosteric modulator depends upon presence of a ligand that would normally activate or inhibit the receptor. Other compounds may activate by inhibiting the action of a molecule that would otherwise lessen or terminate the effect of an endogenous ligand. Still other compounds may activate by increasing the length of time over which an agonist (either endogenous or not) remains bound to the receptor.

B. Enhancement of Synaptic Plasticitv by Decreasing Action Potential Firing

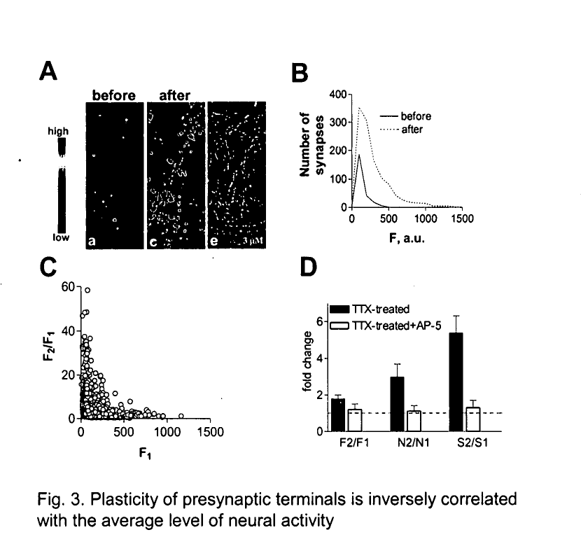

One way to reduce overall activity of a neural network is to reduce action potential firing. As mentioned above, action potential firing involves opening of Na⁺ channels in the cell membrane. The agent TTX is known to block Na⁺ channel opening. As described in Example 4, the inventors applied TTX to cultured neural networks and then tested the plasticity of presynaptic synapses by application of a theta-burst stimulus. It was observed that pretreatment with TTX markedly increased presynaptic plasticity (FIG. 3A). The invention therefore provides a method of enhancing synaptic plasticity in a neural network comprising exposing the neural network to a compound that reduces action potential firing. While TTX is useful for enhancing synaptic plasticity in vitro, it may not be optimal for administration in vivo. For purposes of enhancing synaptic plasticity in vivo, a variety of other agents may be used, including, but not limited to, phenytoin and related compounds.

C. Enhancement of Synaptic Plasticity by Voltage-Dependent Block of NMDA Receptors

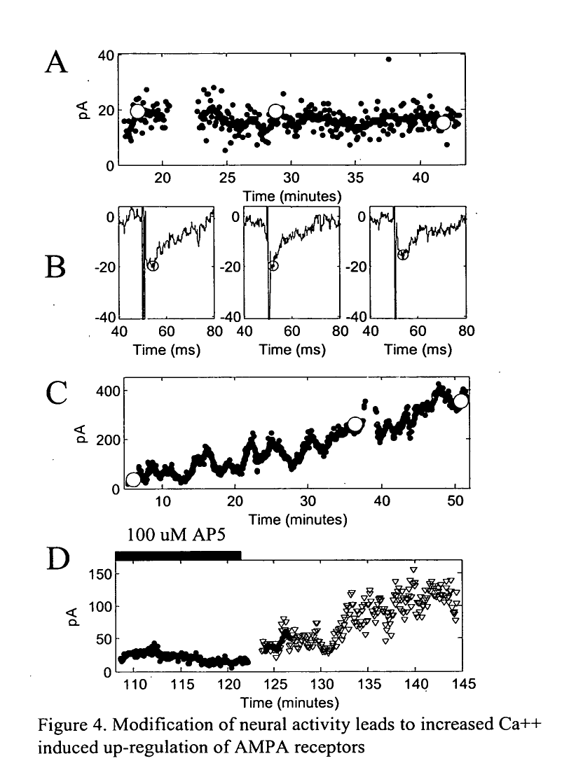

The invention encompasses the recognition that Ca⁺⁺ flux though NMDA receptors and voltage gated Ca⁺⁺ channels provides a signal that links the level of neural activity to the plasticity of synapses. Therefore, inhibiting NMDA receptors at postsynaptic locations would be predicted to lead to an overall reduction in Ca⁺⁺ flux which results in increased synaptic plasticity. This prediction was confirmed by treating neural networks with increased concentrations of Mg⁺⁺. As described above, Mg⁺⁺ produces a voltage-dependent block of NMDA receptors, thereby selectively reducing Ca⁺⁺ influx associated with uncorrelated activity. The block is rapidly relieved during correlated activity. As shown in Example 5, the inventors demonstrated that an increase in the concentration of Mg⁺⁺ in the culture medium resulted in increased responsiveness of postsynaptic spines to Ca⁺⁺ influx (FIGS. 4A and 4B). The inventors further showed that this increase was dependent on the NMDA receptor, as it was not observed in the presence of an NMDA receptor antagonist, while removal of the antagonist restored the effect (FIG. 4C). This experiment demonstrates that reducing the Ca⁺ flux in a neural network enhances postsynaptic plasticity.

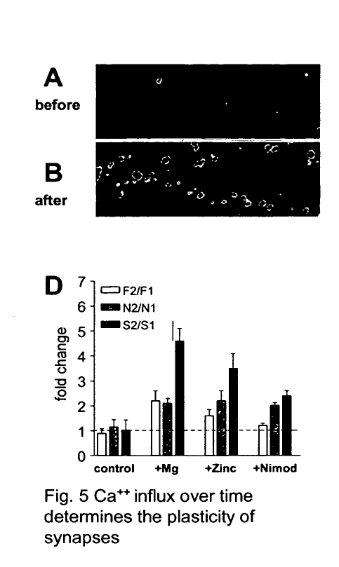

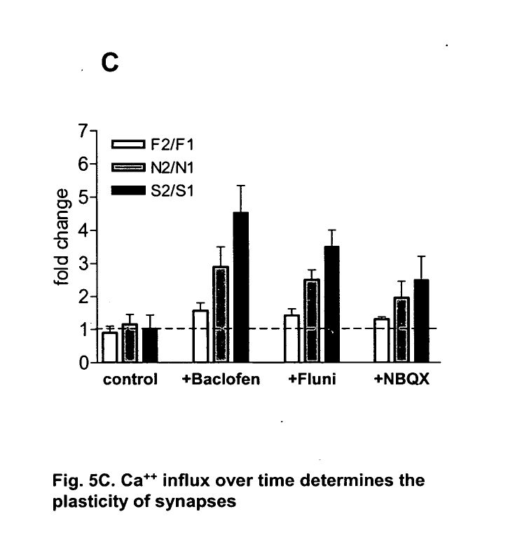

The effect of Mg⁺⁺ was further tested by examining the effect of increased Mg⁺ concentration on presynaptic plasticity. As described in Example 6 and depicted in FIG. 5, Mg⁺⁺ treatment greatly increased the ability of presynaptic terminals to respond to theta-burst stimulation. Thus inhibition of the NMDA receptor by Mg⁺ enhanced presynaptic plasticity in addition to postsynaptic plasticity.

Other ions that will cause a voltage-dependent block of the NMDA receptor include zinc (Zn⁺⁺). As described in Example 6 and depicted in FIG. 5, treatment with Zn⁺⁺ caused similar effects on presynaptic terminals as treatment with Mg⁺⁺, further confirming the efficacy of NMDA receptor block for increasing synaptic plasticity. The invention therefore provides a method of enhancing synaptic plasticity of a neural network comprising exposing the neural network to a compound that inhibits NMDA receptors. In certain embodiments of the invention the composition comprises Mg or Zn. It will be appreciated that these two elements may be provided in a variety of forms, i.e., the Mg⁺⁺ or Zn⁺⁺ cation may be present with any of a number of different anions, e.g., sulfate, citrate, hydroxide, oxide, etc. A wide variety of magnesium and zinc salts are known in the art. In certain preferred embodiments of the invention the compound acts as a voltage-dependent inhibitor of the NMDA receptor, i.e., it inhibits NMDA receptor activation when the membrane in which the receptor is located is at approximately resting potential, but the inhibition is relieved when the membrane is depolarized beyond a certain threshold, e.g., the membrane potential at which Mg⁺⁺ is normally released from the NMDA receptor (˜0 mV). Voltage-dependent inhibitors may be preferred since they will not interfere too greatly with overall functionality of the neural network and provide long term enhancement of synaptic plasticity and cognitive function. Preferably the compound acts at postsynaptic locations at excitatory synapses.

Compounds such as Mg⁺⁺ that cause a voltage-dependent block of NMDA receptors also cause a long term increase cognitive function in vivo. As described in Example 6, rats that received Mg⁺⁺ supplementation in their drinking water over a 4 month period displayed increased expression of a protein whose overexpression has been shown to improve memory. In addition, as described in Example 9, aged rats treated with Mg⁺⁺ displayed enhanced cognitive function as compared with controls. The invention therefore provides a method of increasing cognitive function comprising administering a composition comprising a compound that imposes a voltage-dependent block on an NMDA receptor to a subject. In preferred embodiments of the invention the voltage-dependent block is rapidly relieved during correlated activity, i.e., is rapidly reversible. For example, preferably the compound exhibits the property that the voltage-dependent block is relieved, on the average, within 10 ms of a correlated input from a presynaptic neuron, or within 10 ms of a membrane depolarization. Such a voltage-dependent block will be said to be rapidly reversible. In certain embodiments of the invention the compound exhibits the property that the voltage-dependent block is relieved, on the average, within 5 ms of a correlated input from a presynaptic neuron, or within 5 ms of a membrane depolarization. In certain embodiments of the invention the compound exhibits the property that the voltage-dependent block is relieved, on the average, within 3 ms of a correlated input from a presynaptic neuron, or within 3 ms of a membrane depolarization. In certain embodiments of the invention the compound is able to impose a voltage-dependent block under normal physiological conditions, e.g, under conditions that exist at a synpase in a normal individual, as well as being able to impose a voltage-dependent block under pathological conditions, e.g., in an individual suffering from a condition associated with a cognitive deficit. In certain embodiments of the invention the compound is able to impose a voltage-dependent block under conditions in which excitotoxicity (e.g., glutamate excitotoxicity) does not exist, e.g., under conditions of normal glutamate release and of normal depolarization levels.

In a preferred embodiment of the invention the composition comprises magnesium. Magnesium may be present in the composition in the form of a salt. In solution, such a salt will typically dissociate into ions (e.g., Mg⁺⁺). Compounds that dissolve or dissociate to yield Mg⁺⁺ are said to provide Mg⁺⁺. In general, any pharmaceutically acceptable form of magnesium or a magnesium salt may be used. In certain embodiments it is preferred to use a magnesium salt that is readily bioavailable, i.e., is readily absorbed and available to be used or stored by the subject. Magnesium compounds that are readily soluble in an aqueous medium may be preferred. Examples of such salts include, but are not limited to, MgCl₂, Mg lactate, Mg citrate, Mg aspartate, Mg glycinate, Mg chelazome® (available from Albion Labs), and MgSO₄. In certain embodiments of the invention the solubility and/or bioavailability of the magnesium compound is at least as great as that of MgSO₄. Magnesium may be provided as a complex or chelate with an organic molecule, e.g., an amino acid.

In general, magnesium may be delivered using a solid dosage form (e.g., tablet, caplet, or the like) or a liquid dosage form, as discussed further below. A solid dosage form may comprise a liquid interior and a solid exterior. It may be preferable to utilize a liquid dosage form. Widely used beverages that people consume in their daily lives, e.g., water, fruit or vegetable juices, sodas, etc., may be supplemented with magnesium. The invention encompasses containers (e.g., bottles, cans) for such supplemented beverages that are specifically labeled to indicate that the contents contain magnesium for enhancement of cognitive function, e.g., learning and/or memory, and/or for prevention of diseases such as Alzheimer's disease. A container suitable for holding a liquid is referred to herein as a vessel. The invention also encompasses containers (e.g., bottles) for solid dosage forms (e.g., tablets, caplets, etc.) that are specifically labeled to indicate that the contents contain magnesium for enhancement of cognitive function, e.g., learning and/or memory, and/or for prevention of diseases such as Alzheimer's disease.

As described in Example 9, treatment with magnesium that resulted in an increase in CSF [Mg] concentration from 1.06±0.03 mM to 1.19±0.05 mM, i.e., an increase of approximately 0.13 mM, resulted in increased cognitive function in rats. In humans, the [Mg] in CSF is reported to be approximately 1-1.2 mM. In certain embodiments of the invention a sufficient amount of magnesium is administered to increase the CSF [Mg] by between 0.05 mM and 0.5 mM. In other embodiments of the invention a sufficient amount of magnesium is administered to increase the CSF [Mg] by between 0.05 mM and 0.3 mM. In yet other embodiments of the invention a sufficient amount of magnesium is administered to increase the CSF [Mg] by between 0.05 mM and 0.2 mM, or between 0.1 mM and 0.2 mM. In certain embodiments of the invention a dose of from 1-20 mg/kg/day Mg is administered. In other embodiments of the invention a dose of from 5-10 mg/kg/day is administered. In other embodiments of the invention between 200 mg/day and 400 mg/day is administered. In certain embodiments of the invention less than 800 mg/day in total is administered. It is to be understood that these ranges are merely representative, and appropriate doses may vary based, for example, on the desired biological effect and particular condition being treated and/or on factors such as patient age, diet, etc. The composition may be administered for different time periods, e.g., at least 2 weeks, 4 weeks, 3 months, 6 months, 1 year, or more. In certain embodiments of the invention the composition comprises a GABA_{B} activator in addition to a compound that imposes a voltage-dependent block on NMDA receptors. In certain embodiments of the invention the composition does not comprise memantine.

D. Enhancement of Synaptic Plasticity by Inhibiting AMPA Receptors

As mentioned above, glutamate acts as an excitatory neurotransmitter at AMPA receptors. Therefore, the inventors hypothesized that inhibiting AMPA receptors at postsynaptic locations would lead to an overall reduction in neural activity which would result in increased synaptic plasticity. This prediction was confirmed by treating neural networks with the AMPA receptor antagonist NBQX. As described in Example 6, inhibition of AMPA receptors resulted in a greatly increased presynaptic response to theta-burst stimulation, demonstrating that a reduction in activity of the neural network due to inhibition of AMPA receptors resulted in increased presynaptic plasticity. The invention therefore provides a method of enhancing synaptic plasticity in a neural network comprising the step of exposing the neural network to a compound that inhibits AMPA receptors. Preferably the compound acts at postsynpaptic locations of excitatory synapses. The invention further provides a method of enhancing cogntive function in a subject comprising the step of administering a compound that inhibits AMPA receptors to the subject.

E. Enhancement of Synaptic Plasticity by Stimulating GABA_{A} Receptors

As mentioned above, activation of GABA receptors results in inhibition of the neuron in which the receptors are located. The inventors hypothesized that stimulating GABA_{A} receptors at postsynaptic locations would lead to an overall reduction in neural activity which would result in increased synaptic plasticity. This prediction was confirmed by treating neural networks with the GABA_{A} receptor modulator flunitrazepam. As described in Example 6, the synapses in the neural networks treated with flunitrazepam, which prolongs GABA_{A} receptor activation and enhances inhibitory synaptic transmission, become plastic and increase presynaptic potentiablity to theta-burst stimulation. This result demonstrates that a reduction in activity of the neural network due to increased inhibition by postsynaptic GABA_{A} receptors (caused by application of a GABA_{A} receptor agonist) resulted in increased presynaptic plasticity (FIG. 5C). The invention therefore provides a method of enhancing synaptic plasticity in a neural network comprising the step of exposing the neural network to a compound that stimulates GABA_{A} receptors. Preferably the compound acts at postsynaptic locations of excitatory synapses. Flunitrazapam and a variety of other agents including other benzodiazepine compounds are known to enhance the effects of GABA by prolonging the duration of GABA_{A} channel opening in response to GABA. Any of these compounds are useful in the practice of the present invention, as are compounds that activate GABA_{A} receptors by other mechanisms. The invention further provides a method of enhancing cogntive function in a subject comprising the step of administering a compound that activates GABA_{A} receptors to the subject. Numerous GABA_{A} receptor agonists are known in the art, and any of these compounds can be used.

F. Enhancement of Synaptic Plasticity by Stimulating GABA_{B} Receptors

GABA_{B} receptors are present on the presynaptic location at excitatory synapses, among other locations. The inventors hypothesized that stimulating GABA_{B} receptors would reduce the likehood of releasing neurotransmitter in excitatory synapses, leading to an alteration in the pattern of transmitter release, which would result in increased synaptic plasticity and enhanced cognitive function. The prediction that stimulating GABA_{B} receptors at presynaptic locations would result in increased synaptic plasticity was confirmed by treating neural networks with the GABA_{B} receptor agonist baclofen. As described in Example 6, stimulation of GABA_{B} receptors resulted in a greatly increased presynaptic response to theta-burst stimulation, demonstrating that an alteration in neurotransmitter release due to increased presynaptic inhibition by presynaptic GABA_{B} receptors (caused by application of a GABA_{B} receptor agonist) resulted in increased presynaptic plasticity (FIG. 5C). The invention therefore provides a method of enhancing synaptic plasticity in a neural network comprising the step of exposing the neural network to a compound that activates GABA_{B} receptors. Preferably the compound acts at presynaptic locations of excitatory synapses. Preferably the compound selectively activates GABA_{B} receptors (versus, for example GABA_{A} and/or GABA_{C} receptors). In certain embodiments of the invention the compound is substantially inactive at GABA_{A} and/or GABA_{C} receptors. For example, the ability of the compound to activate GABA_{B} receptors may be at least 5-fold, at least 10-fold, at least 25-fold, at least 50-fold, at least 100-fold, or greater, than the ability of the compound to activate GABA_{A} and/or GABA_{C} receptors, where a higher binding affinity indicates a greater strength of binding. The binding affinity of the compound for GABA_{B} receptors may be at least 5-fold, at least 10-fold, at least 25-fold, at least 50-fold, at least 100-fold, or greater, than the binding affinity for GABA_{A} and/or GABA_{C} receptors.

Furthermore, since the action of presynaptic modulators is often dependent on the activty level of their target synapses (e.g., they inhibit transmitter release at low frequencies of stimulation either without affecting or increasing transmittsion at high stimulation frequencies) (Markram and Tsodyks, 1996; Abbott et al., 1997; Isaacson and Hille, 1997; Brenowitz et at al., 1998), suggested to the inventors that activation of presynaptic GABA_{B} receptors would selectively decrease transmission during uncorrelated neural activity, thereby decreasing the component of Ca⁺⁺ influx into excitatory neurons that is attributable to uncorrelated activity. Similarly to treatments that impose a voltage-dependent block on NMDA receptors, such treatments should result in a long term enhancement of synaptic plasticity and cognitive function. As described in Examples 8 and 10, the inventors showed that treatment with the selective GABA_{B} agonist baclofen induced enhancement of synaptic plasticity over both short term (4-6 hours) and long term (48 hours) periods of time. Moreover, baclofen triggered increased sensitivity of EPSC_{NMDA} to ifenprodil, a selective blocker of NR2B subunits, indicating that there is an increase in NR2B-containing NMDA receptors. Aged rats treated with baclofen over a period of 4 months demonstrated significant enhancement of NR2B and GLUR1 proteins, an effect also seen with Mg⁺⁺. As mentioned above, NR2B has been shown to play a major role in synaptic plasticity and cognitive processes. These results indicate that compounds that activate GABA_{B} receptors are useful for increasing synaptic plasticity and for enhancement of cognitive function on a long term basis.

The invention therefore provides a method of enhancing cognitive function in a subject comprising the step of administering a composition comprising a compound that activates GABA_{B} receptors to the subject. Baclofen, related compounds, and any other compounds that stimulate GABA_{B} receptors by a similar mechanism are useful in the practice of the present invention, as are compounds that activate or enhance the activity of GABA_{B} receptors by other mechanisms. Such compounds include GABA_{B} agonists, GABA_{B} partial agonists, and allosteric modulators of GABA_{B} receptors.

Baclofen (4-amino-3-(4-chlorophenyl) butanoic acid) (Swiss patent No. CH 449,046) is a selective GABA_{B} agonist. Its structure is shown below:

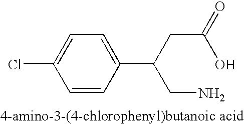

Baclofen is approved by the U.S. Food and Drug Administration and is used clinically for the treatment of spasticity and skeletal muscle rigidity, e.g., in patients with spinal cord injury, multiple sclerosis, amyotrophic lateral sclerosis, and cerebral palsy. Intrathecal administration of baclofen has also shown promise for the relief of chronic pain. Baclofen has also been shown to alleviate addiction, e.g., to drugs of abuse, including nicotine and alcohol dependence.

Additional GABA_{B} receptor agonists or partial agonists are disclosed in EP 0356128; EP 0181833; EP 0399949; EP 0463969; FR 2,722,192; U.S. Pat. Nos. 5,281,747 and 6,117,908. See also Froestl, W. and Mickel, S. J. in: The GABA Receptors, pp. 271-296 (Eds. S. J. Enna and N. G. Bowery, Humana Press Inc., Totowa, N.J., U.S.A. 1997).

In certain embodiments of the invention the GABA_{B} receptor agonist is a substituted aminopropyl acid derivative where the acidic head group is a carboxylic group, a phosphinic group, a phosphonous group or a sulfinic group. Examples of compounds that behave as agonists or partial agonists at GABA_{B} receptors and which can therefore be used according to the invention include, but are not limited to: 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, 3-amino-2-(4-chlorophenyl)-1-nitropropane, (3-aminopropyl)phosphonous acid, (4-aminobut-2-yl)phosphonous acid, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, β-amino-2-(4-chlorophenyl)-2-hydroxypropyl]phosphonous acid, (3-amino propyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methyl phosphinic acid, (3-aminopropyl)hydroxymethylphosphinic acid, (5-aminopent-3-yl)methylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid.

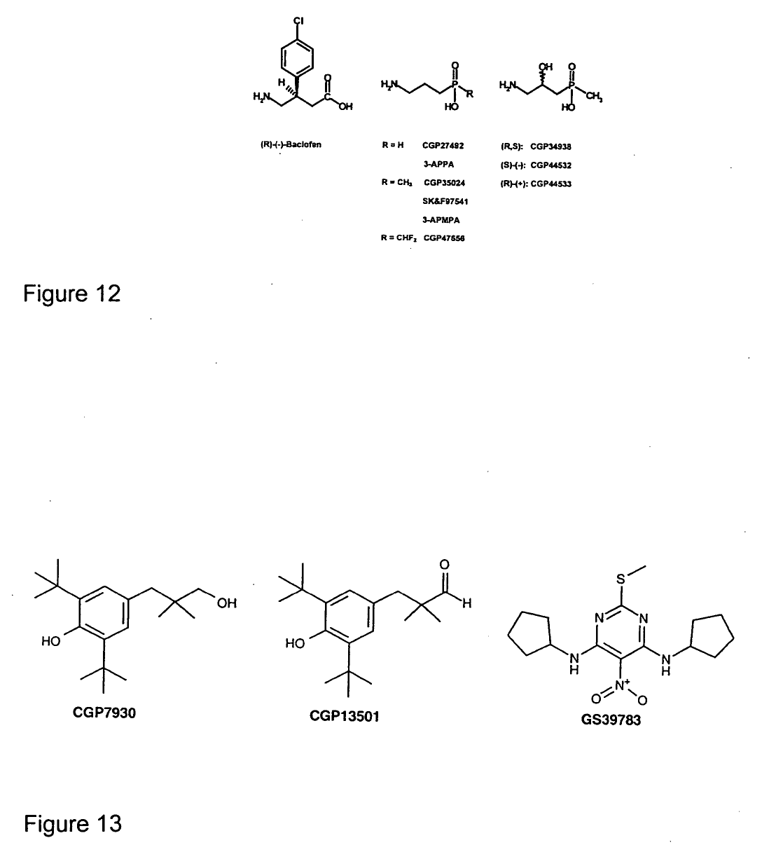

In certain embodiments of the invention the compound is selected from the following: 4-amino-3-(4-chlorophenyl)butanoic acid (baclofen), (3-aminopropyl)methylphosphinic acid, (3-amino-2-hydroxypropyl)methylphosphinic acid, 4-aminobutanoic acid (GABA), (3-amino-2-(4-chlorophenyl)propyl)sulfinic acid, (3-aminopropyl)(difluoromethyl)phosphinic acid, (3-amino-2-oxo-propyl)methyl phosphinic acid, 4-amino-3-(5-chlorothien-2-yl)butanoic acid, and (3-aminopropyl)phosphonous acid. In certain embodiments of the invention 3-aminopropyl-phosphinic acid (3-APPA, also known as CGP27492), or its methyl homolog (3-AMPMA, also known as CGP35024), or other methyl phosphinic acid based agonists such as CGP44532 or its (R)-(+) enantiomer (CGP44533) or racemate (CGP34938) are used. These compounds are pictured in FIG. 12. The difluoromethyl phosphinic acid derivative CGP47656 (FIG. 12) shows partial agonist activity and can also be used.

Many known GABA_{B} activators, e.g., baclofen, (3-aminopropyl)methylphosphinic acid and (3-amino-2-(S)-hydroxypropyl)methylphosphinic acid are amphoteric in nature and may be present in the form of internal salts. They also can form acid addition salts and salts with bases. Pharmaceutically acceptable acid addition salts, as well as pharmaceutically acceptable salts formed with bases, may be used in accordance with the invention. Suitable acids for the formation of such salts include, for example, mineral acids such as hydrochloric, hydrobromic, sulfuric or phosphoric acid or organic acids such as organic sulfonic acids and organic carboxylic acids. Salts of GABA_{B} activators with bases include alkali metal salts, e.g. sodium or potassium salts, alkaline earth metal salts, e.g. calcium or magnesium salts, and ammonium salts, e.g., those with ammonia or organic amines, e.g. diethylamine, di-(2-hydroxyethyl)amine or tri-(2-hydroxyethyl)amine. In certain embodiments of the invention a magnesium salt is used, e.g., a magnesium salt of baclofen. In accordance with the invention, such compounds enhance synaptic plasticity and cognitive function both by imposing a voltage-dependent block on NMDA receptors and by enhancing presynaptic inhibition. Use of these compounds may allow a lower dose of each active agent (e.g., Mg and baclofen), than when the active agents are used individually.

Optical isomers of GABA_{B} activators may be used. A number of the compounds mentioned above, e.g., baclofen and (3-amino-2-(S)-hydroxypropyl)methylphosphinic acid are chiral compounds due to the presence of an asymmetric carbon atom. The GABA_{B} activators may be provided in the form of mixtures of isomers, e.g., racemates, or in the form of pure isomers, e.g., enantiomers. For example, (R)-(−)-baclofen may be used.

GABA metabolites that activate GABA_{B} receptors, or active metabolites of any of the compounds listed above, could also be used. For example gamma-hydroxybutyrate (GHB), an endogenous metabolite of GABA, has been shown to replicate certain therapeutic effects of baclofen with respect to reducing cravings for drugs of abuse. It has been approved by the FDA under the trade name Xyrem for treatment of cataplexy in patients with narcolepsy (U.S. Xyrem Multicenter Study Group, 2004). However, this compound may also have potential to become a drug of abuse itself, and its availability is regulated.

Various compounds that enhance GABA_{B} receptor activity through mechanisms other than acting as GABA_{B} receptor agonists may also be used. For example, γ-vinyl-GABA inhibits the GABA transaminase and thereby elevates GABA levels. Other GABA transaminase inhibitors such as vigabatrin (Angehagen, 2003) or its difluoro-substituted analogue (Pan, 2003), or phenylethylidenehydrazine (Duffy, 2004) could also be used. GABA transport inhibitors such as tiagabine (Angehagen, 2003) or others (Schousboe A, 2004) could also be used. It is noted that GABA transaminase inhibitors and GABA transport inhibitors would also be expected to activate GABA_{A} and/or GABA_{C} receptors. It may be preferable to utilize compounds that selectively activate GABA_{B} receptors, e.g., selective GABA_{B} receptor agonists. Such compounds selectively activate GABA_{B} receptors relative to their effects on GABA_{A} and/or GABA_{C} receptors. A number of methods that can be used to assist in determining whether a compound is a selective agonist are well known in the art. See, e.g., U.S. Pat. No. 6,632,806. The ability of a compound to activate a particular GABA receptor type can be expressed relative to the ability of GABA to activate the receptor. For example, the concentration of the compound necessary to produce half the maximal effect of GABA on the receptor can be determined. In certain embodiments of the invention the concentration of the compound needed to produce half the maximal effect of GABA on GABA_{B} receptors is less than the concentration needed to produce half the maximal effect of GAB_{A} on GABA_{A} and/or GABA_{C} receptors. In other embodiments of the invention the concentration of the compound needed to produce half the maximal effect of GABA on GABA_{B} receptors is less than 0.5 times the concentration needed to produce half the maximal effect of GAB_{A} on GABA_{A} and/or GABA_{C} receptors. In yet other embodiments of the invention the concentration of the compound needed to produce half the maximal effect of GABA on GABA_{B} receptors is less than 0.2, less than 0.1, less than 0.05, less than 0.02, less than 0.01, less than 0.005, or less than 0.001 times the concentration needed to produce half the maximal effect of GAB_{A} on GABA_{A} and/or GABA_{C} receptors. In certain embodiments of the invention the compound is substantially inactive at GABA_{A} and/or GABA_{C} receptors, e.g., does not activate or inhibit GABA_{A} and/or GABA_{C} receptors. For example, the compound preferably does not substantially alter Cl⁻ flux associated with activation of GABA_{A} and/or GABA_{C} receptors by GABA. For example, the amount of Cl⁻ flux that occurs upon application of GABA in the presence of the compound may be within 25%, preferably within 10% or within 5% of the amount of Cl⁻ flux that occurs upon application of GABA alone. The binding affinity of the compound at GABA_{A} and/or GABA_{C} receptors may be 5-fold lower, 10-fold lower, 25-fold lower, 50-fold lower, 100-fold lower, or even less than the binding affinity of the compound at GABA_{B} receptors.

A number of GABA_{B} receptor allosteric modulators have been identified and are of use in the present invention. For example, the compounds CGP7930 [2,6-Di-tert-butyl-4-(3-hydroxy-2,2-dimethyl-propyl)-phenol] and its aldehyde analog CGP13501 were identified as positive modulators of GABA_{B} receptor function (Urwyler, 2001). N,N′-Dicyclopentyl-2-methylsulfanyl-5-nitro-pyrimidine-4,6-diamine (GS39783) and structurally related compounds were also shown to act as allosteric enhancers of GABA_{B} receptor function (Urwyler, 2003). Exemplary compounds are pictured in FIG. 13. They are structurally distinct from GABA_{B} agonists mentioned above and markedly enhance agonist-stimulated responses at GABA_{B} receptors while having little or no intrinsic activity at GABA_{B} receptors. Certain arylalkyl amines, e.g., fendiline (N-[3,3-diphenylpropyl)-alpha-methylbenzylamine) and its congeners, prenylamine (N-[3,3-diphenylpropyl)-alpha-methylphenylethylamine) and F551 (N-[3,3-diphenylpropyl)-alpha-methyl-3-methoxybenzylamine) also behave as positive allosteric GABA_{B} modulators (Kerr, 2002). Selected neutral L-alpha-amino acids, and their dipeptides, were shown to be reversible, stereospecific, potentiators of GABA_{B} receptor-mediated hyperpolarizing responses to baclofen in rat neocortical slices (Kerr, 2003). Most potent were L-Leu, L-Ile and L-Phe and the dipeptides L-Phe-Phe and L-Phe-Leu, and less potent were L-Met, L-Val, L-Cys, L-Cystine, L-Tyr, L-Thr, L-Arg and L-Ser. Any of the afore-mentioned positive GABA_{B} modulators may be used in accordance with the present invention. In certain embodiments of the invention a combination of a GABA_{B} agonist and a positive GABA_{B} receptor modulator, e.g., a positive allosteric modulator, is used.

In certain embodiments of the invention the dose of a GABA_{B} agonist or positive GABA_{B} receptor modulator used for enhancement of cognitive function is less than that used for treatment of other conditions. For example, for reducing spasticity, baclofen/Lioresal® is often started at a dose of 15 mg twice daily (Katzung, 1998, p. 446). Amounts ranging from 30-100 mg/day in divided doses are typically used. Oral dosage forms (tablets) containing 10 mg or 20 mg of baclofen are available. However, the present invention contemplates use of baclofen at doses 10 mg/day or, preferably, less. In certain embodiments of the invention a dose of 5-10 mg/day is used. In other embodiments of the invention a dose of 2-5 mg/day is used. In yet other embodiments of the invention a dose of 1-2 mg/day, or a dose of <1 mg/day is used. For example, a dose of 5 mg/day may be used. In terms of weight, a dose range of 0.02-0.05 mg/kg/day, 0.05-0.1 mg/kg/day, or 0.1-0.15, 0.15-0.2 mg/kg/day may be used. It may be preferable to administer the daily dose as multiple smaller daily doses. For other GABA_{B} agonists, a daily dose having equivalent potency and/or efficacy to that provided by the afore-mentioned dosages of baclofen can be used. Such dosages will be referred to as equipotent. An equipotent dose can be determined using methods known in the art. For example a dose that results in equal therapeutic effect of enhancing cognitive function, for relieving spasticity, or for relieving pain can be used. A dose that has equal potency in evoking one or more physiological effects of baclofen on GABA_{B} receptors can be used, e.g., equal ability to induce decrease Ca⁺⁺ flux into cells that express the receptor, or increase K⁺ conductance of such cells. Published data can be used to establish an equipotent dose. The composition may be administered for different time periods, e.g., at least 2 weeks, 4 weeks, 3 months, 6 months, 1 year, or more. In certain embodiments of the invention the composition further comprises magnesium.

Since it may be inconvenient to divide tablets of 10 or 20 mg into smaller pieces, the invention provides oral dosage forms (e.g., tablets) of baclofen containing 0.5 mg., 1 mg, 2 mg, or 5 mg baclofen. The invention further provides oral dosage forms of other GABA_{B} receptor agonists in amounts sufficient to provide an equivalent potency or efficacy as that provided by 0.5 mg, 1 mg, 2, mg, or 5 mg baclofen, where potency and efficacy are defined according to their accepted meanings in the pharmacological arts. In certain embodiments of the invention baclofen or another GABA_{B} receptor agonist is provided as a magnesium salt.

G. Enhancement of Synaptic Plasticity by Inhibiting Ca⁺⁺ Channels

L-Type Ca⁺⁺ channels are present on both the presynaptic and postsynaptic sides of excitatory synapses. Activation of these channels will increase activity of a neural network by (i) increasing release of excitatory neurotransmitter by the presynaptic location; and/or (ii) increasing the responsiveness of postsynaptic locations to neurotransmitter. Sufficient influx of Ca⁺⁺ could trigger an action potential directly. The inventors hypothesized that inhibiting L-type Ca⁺ channels of excitatory neurons at presynaptic locations, postsynaptic locations, or both, would lead to an overall reduction the integral of Ca⁺⁺ flux, which would result in increased synaptic plasticity. This effect would occur because inhibiting L-type Ca⁺⁺ channels at presynaptic locations reduces the likelihood that they will release excitatory neurotransmitter that will stimulate the postsynaptic location, while inhibiting L-type Ca⁺⁺ channels at postsynaptic locations reduces their Ca⁺⁺ flux directly. The prediction that inhibiting L-type Ca⁺⁺ channels would result in increased synaptic plasticity was confirmed by treating neural networks with the inhibiting Ca⁺⁺ channel inhibitor nimodipine. As described in Example 6, inhibiting L-type Ca⁺⁺ channels resulted in a greatly increased presynaptic response to theta-burst stimulation, demonstrating that a reduction in activity of the neural network due to inhibiting L-type Ca⁺⁺ channels resulted in increased presynaptic plasticity (FIG. 5D).

The invention therefore provides a method of enhancing synaptic plasticity in a neural network comprising the step of exposing the neural network to a compound that inhibits Ca⁺⁺ channels. A large number of Ca⁺⁺ channel blockers are known in the art. In general, it may be preferable to select agents that exhibit some specificity or preference for L-type Ca⁺⁺ channels versus non L-type Ca⁺⁺ channels in order to minimize effects on Ca⁺⁺ channels outside the CNS. However, N and P/Q type Ca⁺⁺ channels are present on presynaptic terminals at excitatory synapases, and inhibition of these channels will also decrease neural activity by decreasing release of excitatory transmitters. Thus it may be desirable to inhibit these channels in addition to or instead of inhibiting L-type channels. Compounds that inhibit the activity of Ca⁺⁺ channels by mechanisms other than blocking the channel may also be used.

H. Enhancement of Synaptic Plasticitv by Stimulating Metabotropic Glutamate Receptors

The metabotropic glutamate receptors mGluR1 and mGluR5 are present on postsynaptic membranes at excitatory synapses. Activation of these receptors leads to release of endogenous cannabinoids, which act as retrograde messengers, diffusing back across the synaptic cleft and inhibiting the activity of presynaptic terminals [Maejima 2001]. This inhibition will in turn reduce the overall level of activity in the neural network, which will in turn lead to increased plasticity. The invention therefore provides a method of increasing synaptic plasticity in a neural network comprising exposing the neural network to a compound that activates mGluR1 receptors, mGluR5 receptors, or both. Preferably the compound activates mGluR1 and mGluR5 receptors on presynaptic excitatory synapses. Preferably the compound activates mGluR1 and mGluR5 receptors selectively, relative to its effects on other glutamate receptors. A number of selective modulators of group I mGluRs are known, such as 3,3′-Difluorobenzaldazine (DFB) [O'Brien 2003]. These or other agents are useful in the practice of the present invention.

I. Enhancement of Synaptic Plasticity by Stimulating Adenosine Receptors

Adenosine A1 receptors are present on presynaptic locations at excitatory synapses. Activation of these receptors inhibits opening of presynaptic Ca⁺⁺ channels. Opening of these channels tends to inhibit Ca⁺⁺ channels in presynaptic terminals, therefore inhibiting release of excitatory neurotransmitter, thus resulting in an overall decrease in activity of the neural network. As described above, decreasing activity in the network results in increased synaptic plasticity. The invention therefore provides a method of increasing synaptic plasticity in a neural network comprising the step of exposing the network to a compound that activates adenosine A1 receptors. Suitable compounds include, for example, the selective A1 receptor agonist N6-cyclopentyladenosine (CPA). However, any of a variety of different activators of A1 receptors may be used.

J. Enhancement of Synaptic Plasticity by Stimulating ACh Receptors

Muscarinic ACh receptors are present at presynaptic locations of excitatory synapses. Stimulation of certain of these receptors inhibits release of neurotransmitters. The effect of inhibiting release of excitatory transmitters will be to alter the pattern of neural activity in the network, thus enhancing synaptic plasticity. In particular, activation of M₂ receptors on presynaptic terminals will inhibit the presynaptic release machinery, which will decrease release of excitatory transmitters, thereby reducing neural activity. In addition, muscarinic ACh receptors are present on inhibitory neurons (e.g., GABAtergic interneurons) in neural networks. Activating muscarinic receptors on these neurons will stimulate their release of GABA [Wu 2003]. Release of GABA will inhibit the postsynaptic excitatory neurons, thereby decreasing overall activity of the network and enhancing synaptic plasticity. Thus compounds that activate muscarinic receptors will increase synaptic plasticity by either or both of two separate mechanisms.

The invention therefore provides a method of increasing synaptic plasticity in a neural network comprising the step of exposing the network to a compound that activates muscarinic ACh receptors. Suitable compounds include, for example, AChE inhibitors, of which a number are known such as tacrine, donepezil, rivastigmine, and galantamine. These agents are already used for the treatment of Alzheimer's disease. However, the mechanism by which they may act has heretofore been unclear. Other suitable compounds include compounds that act to enhance muscarinic receptor activity in other ways, e.g., by binding to the receptors and activating them. Selective activators of the M₂ and/or M₃ receptor may be preferred.

K. Importance of Calcium Flux as a Common Pathway to Enhance Synaptic Plasticity

The inventors have recognized that a number of the methods for enhancing synaptic plasticity described above act to decrease Ca⁺⁺ flux into postsynaptic neurons, to inhibit release of neurotransmitter from presynaptic locations, or both. The fact that a diverse array of treatments, each of which causes a decrease in overall Ca⁺⁺ flux at excitatory synapses, result in increased synaptic plasticity, suggested to the inventors that Ca⁺⁺ flux is a major factor in determining the plasticity of synapses, and thus of neural networks. Indeed several of the treatments described above (increased Mg⁺⁺ concentration, increased Zn⁺⁺ concentration, Ca⁺⁺ channel blocker) do not induce a significant change in overall neural activity within neural networks although they do reduce Ca⁺⁺ flux. For example, under the conditions of increased Mg⁺⁺ concentration described in the Examples, the voltage-dependence of the Mg⁺⁺ block of NMDA receptors is increased. The Ca⁺⁺ flux in resting and sub-threshold conditions is reduced, while Ca⁺⁺ flux triggered by burst stimulation is not affected since the large depolarizations occurring during burst stimulation is sufficient to remove the Mg⁺⁺ block. As described in Example 6, these treatments resulted in enhanced synaptic plasticity while not reducing overall network activity, confirming the important role of Ca⁺⁺ flux.

The Ca⁺⁺ channels discussed above are all located on the plasma membrane of either presynaptic or postsynaptic terminals and regulate Ca⁺ flux from extracellular fluid. However, Ca⁺⁺ flux may also occur through release of Ca⁺⁺ from internal stores. In accordance with the discovery that Ca⁺⁺ flux into presynaptic and/or postsynaptic locations regulates synaptic plasticity, compounds that inhibit release of Ca⁺⁺ from internal stores (e.g., dantrolene) are also useful in the practice of certain embodiments of the present invention. The two main pathways for Ca⁺⁺ release from the ER are via IP₃ receptors (IP₃R) and ryanodine receptors (RyR). Therefore inhibitors of IP₃ receptors or ryanodine receptors, particularly selective inhibitors of neuron-specific forms of these receptors are useful in certain embodiments of the invention. The invention therefore provides a method of increasing synaptic plasticity in a neural network comprising the step of exposing the network to a compound that inhibits IP₃ receptors. The invention further provides a method of increasing synaptic plasticity in a neural network comprising the step of exposing the network to a compound that inhibits ryanodine receptors.

L. Optimal Levels of Neural Activity

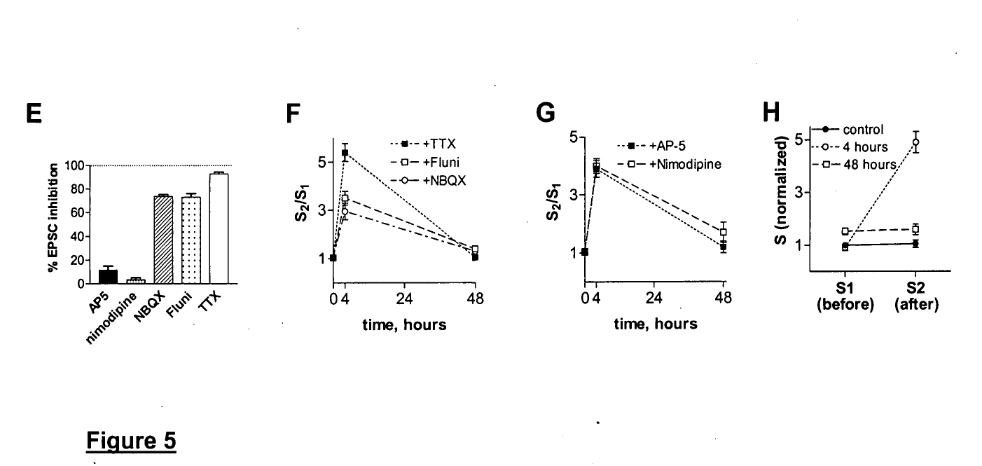

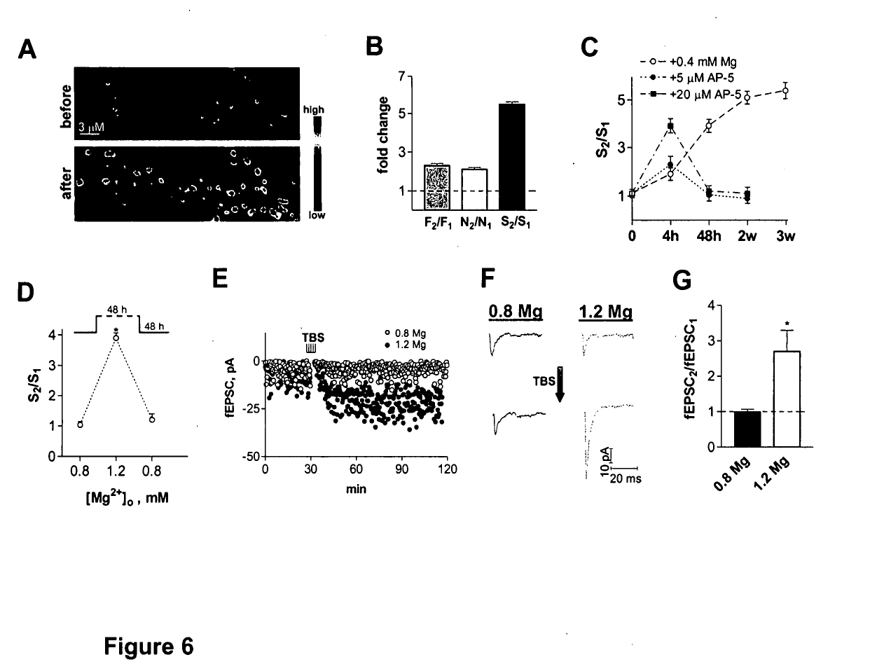

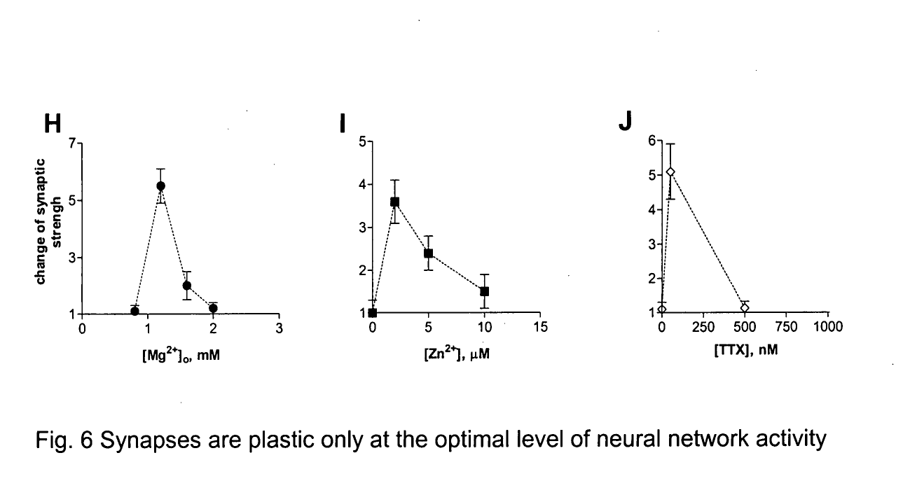

The inventors have recognized that while decreased Ca⁺⁺ flux leads to a reduction in overall activity in a neural network and thus enhances synaptic plasticity, synaptic plasticity may not be highest when neural activity is blocked completely, given the important role that neural activity plays in the functionality of neural networks. The inventors have confirmed the existence of optimal levels of neural activity by showing that there is an optimum concentration of Mg⁺⁺, Zn⁺⁺, or TTX for induction of synaptic plasticity in a cultured neural network (FIGS. 6H-6J). These treatments increased synaptic strength by up to approximately five-fold. Increasing the concentrations of these agents increased synaptic strength up to a certain point, but further increases in concentration resulted in decreased strength. This test may be used to identify optimum compounds, compound combination, and concentrations for maximum induction of synaptic plasticity in vitro or in vivo. However, it is recognized that differences may exist between optimum concentrations in vitro and in vivo.

As described in the Examples, different compounds and concentrations will enhance synaptic plasticity by different amounts. The inventors have recognized that it may not always be desirable to achieve maximum synaptic plasticity in every situation. Accordingly, the compounds and doses may be selected to provide any desired level of enhancement, e.g., an increase of 10%, 20%, 30%, . . . , 100% (two fold), three fold, etc.

In accordance with the findings described above, in certain embodiments of the invention it is preferred to select a treatment (e.g., a compound or compound combination) and/or treatment regimen (e.g., dose and/or dosing schedule) that will result in decreased Ca⁺⁺ flux while having minimal effects on overall neural activity in a neural network. In general, an effective treatment is one that balances the desired effect of enhancing synaptic plasticity while avoiding unwanted reduction in activity of the network.

V. Compounds and Compound Combinations Useful for Enhancing Synaptic Plasticity and Cognitive Function

As described above, a variety of different compounds from a variety of different chemical classes may be used to enhance synaptic plasticity in accordance with the present invention. In general, any compound or combination thereof that decreases Ca⁺⁺ flux into excitatory synapses is of use. In particular, divalent cations (e.g., Mg⁺⁺, Zn⁺⁺), NMDA receptor inhibitors, AMPA receptor inhibitors, mGluR1 activators, mGluR5 activators, GABA_{B} receptor activators, GABA_{A} receptor activators, muscarinic ACh receptor activators including ACHE inhibitors, A1 adenosine receptor activators, voltage-gated Ca⁺⁺ channel inhibitors, and voltage-gated Na⁺ channel inhibitors. Additional compounds that may be used include inhibitors of IP₃ receptors or inhibitors of ryanodine receptors.

Although a wide variety of compounds may be used, the inventors have recognized that use of a single compound may be less than optimal, for a number of different reasons. For example, many of the compounds described above may have a number of undesirable side effects, notwithstanding the fact that a number of them are already used clinically to treat various conditions. In addition, many receptors display a property known as desensitization, which is the progressive inactivation of a receptor due to continuous presence of an activating ligand, e.g., a neurotransmitter, such that the responsiveness of the receptor to the compound is reduced below what it would have been prior to administration of the compound [See, e.g., Jones 1996; Ferguson 2001]. The inventors have recognized that the existence of this phenomenon may limit the efficacy of some of the compounds discussed above.

In order to reduce the likelihood and/or severity of side effects and/or to reduce the development of desensitization, in preferred embodiments of the invention a combination of compounds is administered. Since all the compounds act via a common pathway, i.e., reduction in Ca⁺⁺ flux at excitatory synapses, a large number of different combinations of compounds may be used. Preferably the compounds are administered at doses that do not cause significant desensitization of the target receptor or channel. According to certain embodiments of the invention the compounds are administered at concentrations or doses that do not reduce responsiveness to below 25% of the level observed following a first adminstration of the compound. According to certain embodiments of the invention the compounds are administered at concentrations or doses that do not reduce responsiveness to below 50%, below 75%, or below 90% of the level observed following a first adminstration of the compound. According to certain embodiments of the invention the compounds are administered at concentrations that do not reduce responsiveness to the compound at all. A concentration or dose that causes a reduction in responsiveness to below 50% of the responsiveness observed following a first administration of the compound will be said to cause significant desensitization.

The invention provides a variety of combinations of compounds. As described above, the inventors have identified ten pathways that increase synpatic plasticity by decreasing Ca⁺⁺ flux. These pathways correspond with the following classes of compounds that are useful in the practice of the invention: divalent cations (e.g., Mg⁺⁺ and Zn⁺⁺), NMDA receptor inhibitors, AMPA receptor inhibitors, mGluR1 and/or mGluR5 activators, GABA_{B} receptor activators, GABA_{A} receptor activators, muscarinic ACh receptor activators including AChE inhibitors, A1 adenosine receptor activators, Ca⁺⁺ channel inhibitors, and Na⁺ channel inhibitors. (Note that ACHE receptor activators and AChE inhibitors both act to increase activation of ACh receptors, although the mechanism differs.) Accordingly, the invention provides a composition comprising at least two compounds selected from the group consisting of: divalent cations, NMDA receptor inhibitors, AMPA receptor inhibitors, mGluR1 and/or mGluR5 activators, GABA_{B} receptor activators, GABA_{A} receptor activators, muscarinic ACh receptor activators including ACHE inhibitors, A1 adenosine receptor activators, Ca⁺⁺ channel inhibitors, and Na⁺ channel inhibitors. The invention further provides a composition comprising at least three, at least four, at least five, at least six, at least seven, at least eight, at least nine, or at least ten compounds selected from the group consisting of: divalent cations, NMDA receptor inhibitors, AMPA receptor inhibitors, mGluR1 and/or mGluR5 activators, GABA_{B} receptor activators, GABA_{A} receptor activators, muscarinic ACh receptor activators including ACHE inhibitors, A1 adenosine receptor activators, Ca⁺⁺ channel inhibitors, and Na⁺ channel inhibitors. In certain embodiments of the invention two or more of the compounds may be from the same class while in other embodiments of the invention two or more compounds are from different classes, as further described below. According to certain embodiments of the invention one or more of the compounds is selected from the class of IP₃ receptor inhibitors or the class of ryanodine receptor inhibitors.

In certain embodiments of the invention preferred compound combinations exhibit synergism. In other words, the enhancement of synaptic plasticity provided by the combination of compounds is greater than the sum of the enhancements in efficacy provided by each of the compounds when administered individually. Synergism may result when two compounds each affect different steps in a single process, e.g., activation or inhibition of a single receptor. Synergism may also results when two compounds each affect a different process, and the processes each have a similar ultimate effect. For example, synergism may result when one compound reduces Ca⁺⁺ flux by inhibiting Ca⁺⁺ channels and a second compound reduces Ca⁺⁺ flux by inhibiting the NMDA receptor. In general, each of the classes of compounds listed above acts by a different mechanism to inhibit Ca⁺⁺ flux. Therefore, in certain embodiments of the invention preferred combinations include one or more compounds from at least two, at least three, at least four, at least five, at least six, at least seven, at least eight, at least nine, or all ten of the classes of compounds. Certain preferred compositions comprise Mg⁺⁺ and at least one other compound from a different class. For example, in one embodiment the composition comprises a divalent cation such as Mg⁺⁺ and a GABA_{B} receptor activator (e.g., baclofen). In another embodiment the composition further comprises a voltage-gated Ca⁺⁺ channel inhibitor, or a GABA_{A} receptor activator (e.g., a benzodiazepine). In yet another embodiment the composition comprises an ACHE inhibitor and a divalent cation such as Mg⁺⁺. The composition may further comprise, for example, a voltage-gated Ca⁺⁺ channel inhibitor, a GABA_{B} receptor activator, or a GABA_{A} receptor activator. In certain embodiments of the invention if the composition comprises an NMDA receptor inhibitor and an ACHE inhibitor, then a third compound from a different class is present. In certain embodiments of the invention if the composition comprises an NMDA receptor inhibitor then it does not comprise an AChe inhibitor. In certain embodiments of the invention the composition does not include both an NMDA receptor inhibitor other than Mg⁺⁺ and an ACHE inhibitor. In certain embodiments of the invention the composition does not include both an NMDA receptor inhibitor and an AChE inhibitor. The foregoing examples are included for representative purposes and are not intended to limit the scope of the invention.

In the case of compounds that are already approved for use in humans (e.g., compounds approved by the U.S. Food and Drug Administration or a comparable agency in another country), in certain embodiments of the invention the dose of such a compound in the compositions of the present invention is lower than the minimum recommended therapeutic dose of the compound for the treatment of diseases or conditions in which its clinical efficacy is recognized. For example, the dose of such a compound in the inventive compositions may be less than 90% of the minimum recommended therapeutic dose of the compound, less than 80%, less than 70%, 60%, 50%, 40%, 30%, 20%, 10%, or 5% of the minimum recommended dose. One of ordinary skill in the art will readily be able to determine the minimum recommended dose of any given compound by consulting works such as the Physician's Desk Reference, pharmacology books such as Goodman and Gilman, supra, and/or the scientific literature. Furthermore, it will be appreciated that total dose depends both on the amount of compound that is administered at each dosing and on the interval between doses. Therefore, when a compound useful in the practice of the present invention is administered at a dosing interval that differs from the dosing intervals typically employed when the compound is used for therapy of a disease or condition in which its efficacy has been recognized, the amount of each dose may be adjusted so that the total dose is less than the minimum recommended dose of the compound for that disease or condition.

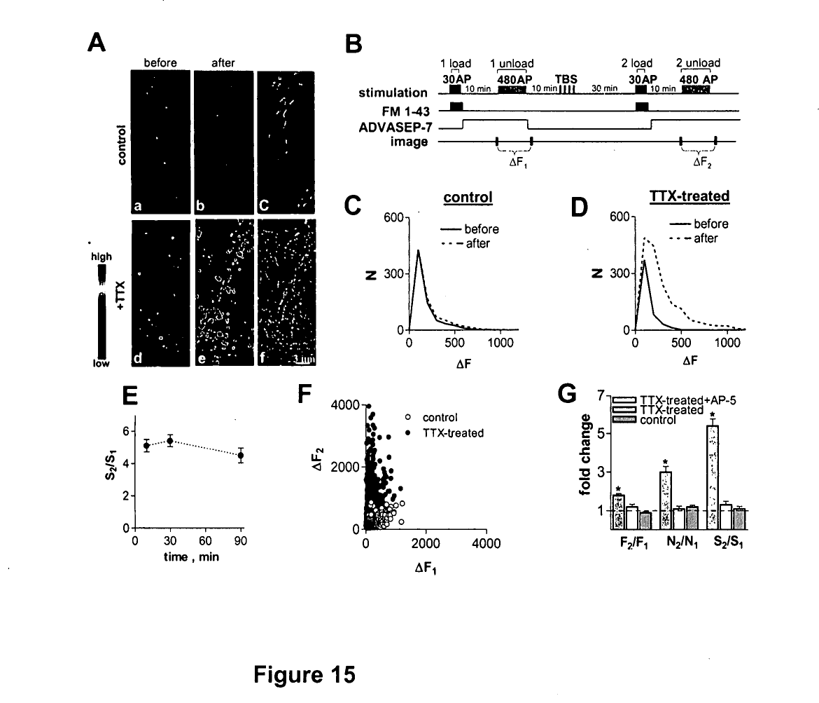

It will further be appreciated that where compositions comprising multiple different compounds are used, it will generally be desirable to limit the total dose of the composition in addition to limiting the doses of individual constituents of the composition. For example, in general, a composition consisting essentially of four different active compounds will contain a lower amount of each compound than a composition consisting essentially of only two different active compounds. The appropriate effective dose (i.e., a dose that will result in enhancement of synaptic plasticity and/or cognitive function) of any given compound or compound combination may be determined in a number of ways. For example, as shown in FIG. 15, there will typically be an optimum dose so that maximum enhancement of synaptic plasticity is achieved. Any particular composition may be tested at a variety of different concentrations in a cultured neural network, and the change in synaptic strength occurring as a result of exposure to the composition may be assessed, e.g., by measuring in order to arrive at an optimum concentration. In general, it may be desirable to achieve such a concentration in the CNS, e.g., in the cerebrospinal fluid. However, it is recognized that differences exist between optimal conditions in a cultured neural network and those in vivo. In general, when the composition comprises a divalent cation such as Mg⁺⁺, the amount of the divalent cation will generally exceed the amount typically used in pharmaceutical preparations in which a magnesium-containing compound (e.g., magnesium stearate) is used as an “inactive” ingredient, i.e., as a bulk material or to improve the material properties of the pharmaceutical preparation rather than for its therapeutic effect.

As noted above, in certain embodiments of the invention the compounds that activate a receptor or channel correspond with those referred to as “agonists” in the literature, while those that inhibit a receptor or channel correspond with those referred to as “antagonists” in the literature, as those terms are commonly understood in the art (See, e.g, Goodman and Gilman, supra). Compounds may act functionally as activators or inhibitors of a receptor or channel, even if they do not bind to the receptor or channel. For example, such compounds may act indirectly to activate or inhibit a receptor or channel by activating or inhibiting a second messenger system whose activation or inactivation results in a modification to the receptor or channel, or results in upregulation or downregulation of the receptor or channel, or they may act to inhibit degradation of an endogenous neurotransmitter (e.g, ACHE inhibitors). Certain compounds act as activators or inhibitors by binding to a receptor or channel, wherein binding results in increased or decreased activity of the receptor or channel. For example, compounds commonly referred to as blockers typically act by physically occluding a channel, thereby preventing flux of ions through the channel. The effect of binding may be to directly activate the receptor or channel, e.g., by causing a change in the structure of the receptor. Binding of the compound may act by increasing or decreasing the efficacy of another compound, e.g., an endogenous compound, to activate or inhibit the receptor or channel. One of ordinary skill in the art will readily be able to determine whether a given compound falls within the classes of compounds disclosed herein as useful for enhancing synaptic plasticity and cognitive function.

VI. Screening Methods

As described in the Examples, the inventors developed novel methods for the observation of synapses and the measurement of synaptic plasticity. These methods are useful to screen for additional compounds that enhance synaptic plasticity and/or cognitive function. Accordingly, the invention provides a method of screening a compound comprising: (i) exposing neurons in a cultured neural network to a detectable substance, wherein the substance is taken up by presynaptic terminals that release neurotransmitter; (ii) exposing neurons in the neural network to the compound; (iii) administering a pattern of stimulus to the neurons in the network; (iv) measuring synaptic plasticity; and (v) identifying the substance as an enhancer of synaptic plasticity and/or cognitive function if the measured synaptic plasticity increases following exposure to the compound. In certain embodiments of the invention synaptic plasticity is measured by detecting presynaptic terminals that have taken up the detectable substance and comparing the synaptic strength before and after a stimulus such as theta-burst stimulation.

In general, an increase in synaptic strength may be identified by comparing the measured synaptic strength with that obtained under control conditions in which the compound is absent. The control conditions may be a historical control; thus it is not necessary to perform controls with each screen. An increase in synaptic strength may also be identified by measuring synaptic strength in response to a stimulus prior to exposure of the neural network to the compound and then measuring synaptic strength in response to a stimulus after the neural network has been exposed to the compound and comparing the values obtained. The neural network may be exposed to the compound for periods of time ranging from minutes or hours to days or weeks.

The detectable substance may be, for example, a fluorescent molecule such as the FM 1-43 or AM 1-43 dyes described in the Examples. However, any detectable substance that is taken up by presynaptic neurons in an activity-dependent manner (i.e., whose uptake correlates with release of neurotransmitter) may be used. In certain embodiments of the invention images are gathered of the cultured neural network of a portion thereof, and analyzed by image processing software in order to quantify synaptic strength. One of ordinary skill in the art will readily be able to write appropriate software routines to gather and process such images.

In certain embodiments of the invention the cultured neural network comprises hippocampal neurons, e.g., hippocampal pyramidal cells. In general, a stimulus appropriate for the type of neurons in the culture is selected. For example, in the case of hippocampal neurons, a theta-burst stimulus protocol may be applied. At later time points, one or more action potentials, e.g., a train of action potentials such as those described in the examples, may be applied. Synaptic strength may be measured by assessing the number of presynaptic terminals that take up the detectable substance, the average amount of detectable substance taken up by presynaptic terminals, or both. A combined measure of presynaptic strength, e.g., a product of the foregoing, may conveniently be used. Postsynaptic strength may be measured instead of, or in addition to, presynaptic strength.

The screening methods described above offer a number of advantages in that they are rapid and quantitative. Compounds identified using the inventive screening methods may be further tested in a variety of animal models that are commonly employed in the study of learning and memory and in screens to identify compounds of use in the treatment or prevention of memory impairment. Certain appropriate tests are described in [Tang 1999] and include novel-object-recognition tasks, contextual and cued fear conditioning, fear-extinction, and spatial learning (e.g., performance in the hidden-platform water maze also known as the Morris water maze). Additional suitable tests are described in U.S. Pat. No. 6,632,806. For example, the ability of a candidate compound to enhance memory may be tested in mice using a plus-maze memory test. The ability of a candidate compound to enhance memory may be tested in chicks using, e.g., a taste discrimination test. In addition, the compounds may be further screened in humans using, for example, any of a variety of tests of memory and/or learning ability such as are widely used in psychology and medicine, e.g., the Clinician's Interview-Based Impression of Change Plus Caregiver Input (CIBIC-Plus), the Alzheimer's Disease Cooperative Study Activities of Daily Living Inventory modified for severe dementia (ADCS-ADLsev), the Severe Impairment Battery, etc. [Reisberg 2003], and various other tests of cognitive function mentioned above. Diagnosis of a condition associated with cognitive impairment, e.g., Alzheimer's disease, dementia, etc., may be performed in accordance with diagnostic criteria set forth in Diagnostic and Statistical Manual of Mental Disorders DSM-IV-TR (Text Revision) American Psychiatric Association; 4th edition (June 2000).

Compounds suitable for screening include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. One of ordinary skill in the art will readily be able to identify further sources of compounds to screen. In addition, one of skill in the art of medicinal chemistry will be able to modify compounds in the ten classes discussed above to develop analogs that exhibit superior ability to enhance synaptic plasticity.

VII. Therapeutic Applications

As discussed above, synaptic plasticity is believed to be essential for the processes involved in learning and memory. Thus compositions that enhance synaptic plasticity are of use for the treatment of individuals (subjects) suffering from any of a variety of conditions in which cognitive function, e.g., memory and/or learning is impaired. The compositions are also useful to prevent the onset of such conditions. These conditions include, but are not limited to, those known as “benign senescent forgetfulness”, “age-associated memory impairment”, “age-associated cognitive decline”, “mild cognitive impairment”, Alzheimer's disease, dementias (associated with any of a number of causes), attention-deficit disorder, etc. The compositions and methods of the invention may also find use to enhance the cognitive function, e.g., memory and/or learning capacity of normal individuals, i.e., individuals not suffering from any clinically recognized condition or disorder. They may be useful on a short-term basis or may be administered chronically. They may be administered daily, multiple times per day, or at intervals greater than a day. It may be desirable to administer the compositions in the evening, prior to sleep for a number of reasons. For example, certain of the compounds are sleep-inducing and their use may interfere with waking activity. In addition, since sleep is a state associated with increased inhibition in the CNS, the compounds may be more efficacious if they are present in the CNS during sleep and/or if they reach a peak concentration in the CNS during this time.

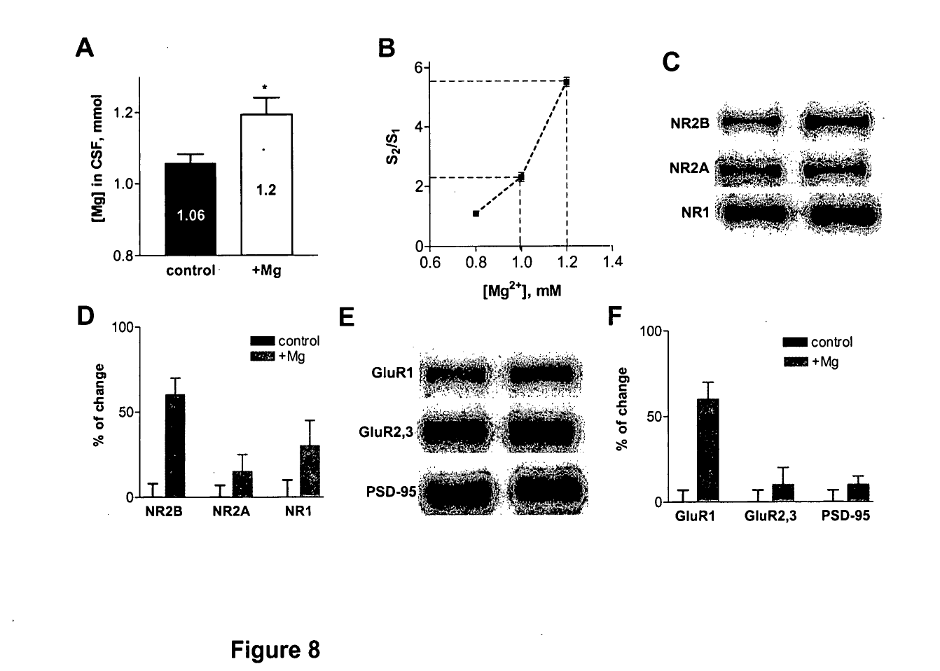

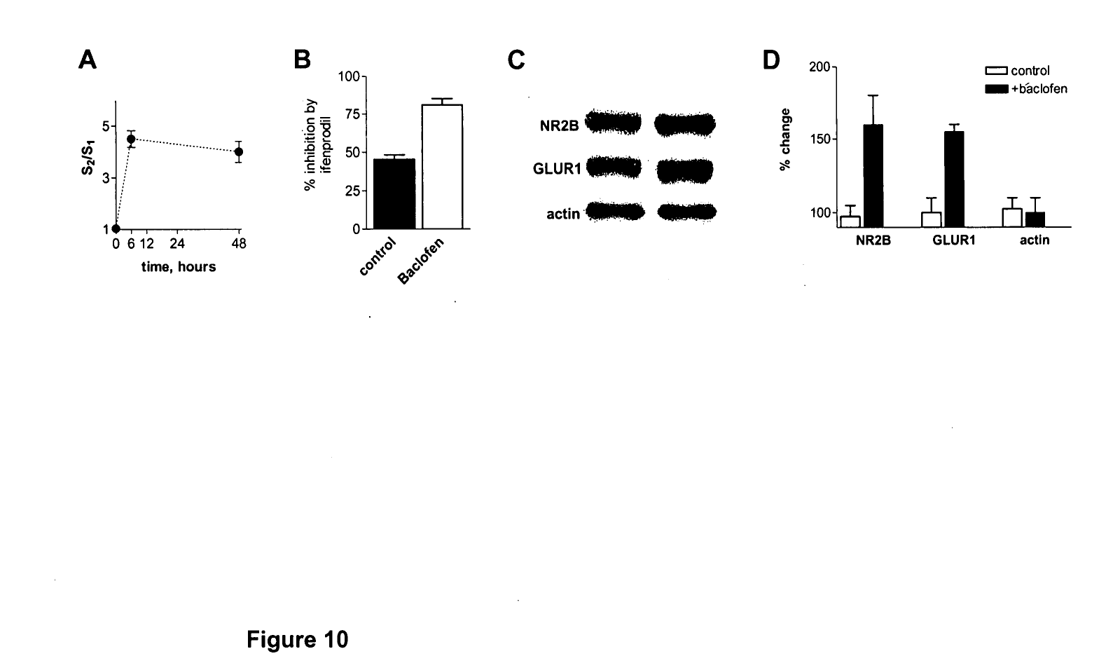

The utility of the compositions and methods of the present invention for enhancing memory was tested in “elderly” mice. As mentioned above and described in Example 9, the inventors administered Mg⁺⁺ in the drinking water of 8 month old rats for a period of approximately 4 months. They then examined the level of the NR2B subunit of the NMDA receptor in the brains of these rats. It was found that Mg⁺⁺ treatment resulted in a two-fold increase in levels of NR2B protein (FIG. 8). Similarly, treatment with a GABA_{B} agonist, baclofen, resulted in an approximately 60% increase in levels of NR2B protein (FIG. 10). It has previously been found that transgenic mice engineered to overexpress this protein in the forebrain exhibit superior ability in learning and memory in various behavioral tasks [Tang 1999]. Furthermore, it has been found that patients with Alzheimer's disease have a reduced level of NMDA receptor proteins, including NR2B [Sze 2001]. Therefore, not only is overexpression of NR2B associated with improved learning and memory, but loss of the protein is associated with AD, in which memory impairment is a major symptom. In addition, as described in Example 9, treatment with Mg⁺ improved performance of aged rats in a test of cognitive function, i.e., a novel object recognition test. Cognitive function testing with baclofen is ongoing. The fact that long term treatment with Mg⁺⁺ or a GABA_{B} agonist results in a doubled level of the NR2B protein and that treatment with Mg⁺⁺ increased performance in a test of cognitive function demonstrates the utility of the compositions and methods of the present invention for use in enhancing memory and/or learning in mammalian subjects, e.g., rodents, primates, etc., particularly human beings, and for treating or preventing conditions associated with memory impairment, e.g., Alzheimer's disease.

In addition, it has been observed that in its early stages Alzheimer's disease characteristically features an impairment of memory and a relative absence of other symptoms of cognitive malfunction that typically occur later in the disease. Mounting evidence suggests that this syndrome begins with subtle alterations of hippocampal synaptic efficacy prior to frank neuronal degeneration [Selkoe 2002]. This evidence therefore further indicates the utility of the compositions and methods of enhancing synaptic plasticity described herein for treatment of AD.

Accordingly, the invention provides a method of treating memory impairment comprising the steps of: (i) providing a subject at risk of or suffering from a condition associated with memory impairment, dementia, or cognitive deficit; and (ii) administering to the subject any of the inventive compositions discussed above. The compositions may be administered for short periods of time such as days or a few weeks, e.g., to provide short term enhancement of learning ability or memory. However, it is anticipated that the inventive compositions may be administered on a chronic basis, e.g., for many weeks, for months, for years, or indefinitely. The subject may be suffering from or at risk of memory impairment from any of a variety of causes. In particular, the subject may be at risk of or suffering from age-associated memory impairment, mild cognitive impairment, or Alzheimer's disease. A variety of criteria may be used to determine whether or not a particular individual suffers from any of the foregoing conditions. See, e.g., discussion in [Petersen 2001; Burns 2002; Clark and Karlawish 2003; and Karlawish and Clark 2003] and references listed in these articles. In particular, AD may be diagnosed according to the National Institute of Neurological and Communicative Disorders and Stroke—Alzheimer's Disease and Related Disorders Association criteria for a clinical diagnosis of probable Alzheimer's disease. Imaging and various biomarkers (e.g., levels of tau protein in cerebrospinal fluid). In addition, individuals with dominant mutations in the amyloid precursos protein, PS1, or PS2 genes are at increased risk of AD. It has also been found that the risk of developing AD is greater in individuals with the ε4 allele of the gene encoding ApoE. Such individuals may be particularly appropriate candidates for therapy with the compositions described herein.

VII. Pharmaceutical Compositions

Inventive compositions may be formulated for delivery by any available route including, but not limited to oral, parenteral, intradermal, subcutaneous, by inhalation, transdermal (topical), transmucosal, rectal, and vaginal. The term “parenteral” as used herein includes subcutaneous, intravenous, intramuscular, intra-articular, intra-synovial, intrasternal, intrathecal, intrahepatic, intralesional and intracranial injection or infusion techniques. In general, oral administration is preferred. Inventive pharmaceutical compositions typically include one or more compounds of the classes discussed above in combination with a pharmaceutically acceptable carrier, adjuvant, or vehicle. As used herein the language “pharmaceutically acceptable carrie, adjuvant, or vehicle” includes solvents, dispersion media, coatings, antibacterial and antifungal agents, isotonic and absorption delaying agents, and the like, compatible with pharmaceutical administration. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, antibacterial and antifungal agents, isotonic and absorption delaying agents, and the like, compatible with pharmaceutical administration may be included. Supplementary active compounds, e.g., compounds independently active against the disease or clinical condition to be treated, or compounds that enhance activity of an inventive compound, can also be incorporated into the compositions.

Further provided are pharmaceutically acceptable compositions comprising a pharmaceutically acceptable derivative (e.g., a prodrug) of any of the compounds of the invention, by which is meant any non-toxic salt, ester, salt of an ester or other derivative of a compound of this invention that, upon administration to a recipient, is capable of providing, either directly or indirectly, a compound of this invention or an active metabolite or residue thereof. As used herein, the term “active metabolite or residue thereof” means that a metabolite or residue thereof is also able to enhance cognitive function and/or synaptic plasticity.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, chloride, bromide, iodide, hydrochloride, hydrobromide, hydroiodide, hydroxide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. A parenteral preparation can be enclosed in ampules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive compositions are preferably delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from neuronal culture assays. A dose can be formulated in animal models to achieve a circulating plasma concentration range that includes the IC₅₀ (i.e., the concentration of the test compound which achieves a half-maximal inhibition of symptoms) as determined in neuronal cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition as described herein, can include a single treatment or, in many cases, can include a series of treatments.

Exemplary doses include milligram or microgram amounts of the inventive composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

In the case of many of the compounds described herein, guidance in determining appropriate maximum doses for is available since these compounds are already used in therapy of other conditions. As noted above, however, it will frequently be desirable to employ lower doses. In addition, guidance in determining maximum doses may be obtained by constructing a dose response curve based on results in cultured neural networks or intact animals.

Inventive pharmaceutical compositions can be included in a container, pack, or dispenser together with instructions for administration.

### EXAMPLES

### Example 1

### Detection of Presynaptic Function Using FM Dye Staining

Materials and Methods

Cover Slip Method for Postnatal Rat Hippocampal Neuron Culture

The protocol below describes culture methods used for all examples except as otherwise noted.

Solutions:

Basic Medium: MEM, Gibco 51200-038 (No phenol red)

500 ml of MEM
2.5 g glucose
100 mg NaHCO3
50 mg transferrin, Calbiochem 616420
   filter-sterilize. Store at 4° C.
   Plating Medium: On the day of use take 89 ml of the above MEM/Basic Medium and add:
30 mg glutamine [1 ml 0.2 M glutamine stock/100 ml medium, store glutamine at −20° C.]
2.5 mg insulin Sigma 15500 [0.2 ml of insulin stock (12.5 mg insulin/1 ml 10 mM HCl)/100 ml medium, store insulin at −20° C.]
10 ml fetal bovine serum (FBS) [if not done, heat inactivate serum at 57° C. for 30 min., stored at 4° C.]
   Filter-sterilize. For 15 plates, equilibrate about 35 ml of medium in incubator (37° C., 5% CO₂)
   Feeding medium: Make fresh weekly and keep on hand for feeding.
97 ml of Basic Medium
1 ml of glutamine stock
2 ml of B-27 stock [Gibco 17504-044, 50× concentration]
300 μl of ARA-C stock [1 mM ARA-C, cytosine arabinoside, Sigma C6645, sterile]
   Filter-sterilize.
   Hank's Salt Solution: Hank's balanced salt solution (HBSS) without calcium or magnesium (Sigma H2387)
350 mg/L NaHCO3 (4 mM)
1.3 g/L HEPES (5 mM)
   pH solution to 7.3-7.4 and filter-sterilize. Wrap the lid with parafilm to help maintain pH of solutions.
   HBSS/20% FBS.

Make up 50 ml at a time on the day of the dissociation.

Digestion Solution:

| | |
|---|---|
| 137 mM NaCl | 400 mg/50 ml |
| 5 mM KCl | 18 mg/50 ml |
| 7 mM Na2HPO4 | 50 mg/50 ml |
| 25 mM HEPES | 150 mg/50 ml |

pH to 7.2 and store at 4° C. Solution may be sterilized for storage purposes but will also be sterilized at time of use.
Dissociation Solution
Add 150 mg of MgSO2*(7H2O) to 50 ml of HBSS [12 mM].
   DNase and Trypsin Stocks
DNase type IV, Sigma D5025 1 mg Dnase/50 μl H2O stored at −20° C.
Trypsin type XI, Sigma T1005 10 mg trypsin/200 μl H2O stored at −20° C.
   All solutions should be at 4° C., i.e. on ice, during the procedure, except plating medium, which should be at 37° C.
   Materials Preparation
   Coverslips:
   Circular glass coverslips, 12 mm size, No. 0 thickness, Carolina Biological Supply P-763-3009.

Using sonication, wash coverslips in xylene for ˜30 min. Repeat wash with 100% ethanol (3×). Rinse at least 5 times with distilled de-ionized water. Separate coverslips manually and allow them to air-dry before transferring to a glass petri dish and autoclaving (dry cycle 20 min sterilize/60 min dry).

Generally, use 15 dishes [2 coverslips/35 mm dish] for each prep [3 rat pups/prep]. Using sterile technique, transfer coverslips to dishes and dot with ˜0.1 ml diluted Matrigel [Collaborative Research Inc., Bedford, Mass.]. Aliquotes of Matrigel (˜50 μl) are kept at −20° C. Thaw on ice and dilute 1:100 in sterile, cold MEM. If the temperature of Matrigel increases above 4° C., it will “polymerize”. Incubate coverslips with Matrigel at 37° C. while proceeding with cultures. Just before use, aspirate Matrigel gently and plate cells directly onto coverslips.

Siliconized Pasteur Pipettes:

Coat the inside of Pasteur pipettes by drawing up a solution of 0.2% silicon oil/ether (Aldrich 14,615-3). Rinse out several times with dd-water. Air-dry, plug with cotton, and autoclave on dry cycle.

Odds and Ends:

Autoclaved, not plugged, 9 inch Pasteur pipettes.

Dissecting instruments in excellent condition, sterilized by soaking in 70% ETOH for >30 min.

Animals

Neonatal Sprague-Dawley rats, post-natal day 1; use 4 pups/preparation. The original protocol used 2-3 day old animals. The younger the animals are the fewer glia in the prep.

Dissection

1. Decapitate animal and remove the brain into a 60 mm petri dish containing ice-cold HBSS/20% FBS. Complete dissection before proceeding to next animal.
2. Hemi-dissect brain, removing brainstem and thalamus to expose hippocampus. Remove hippocampus leaving some of the subiculum attached. Tug and pull as little as possible. Always cut, not crush, tissue.
3. Transfer hippocampi to a second 60 mm dish of cold HBSS/20% FBS. Use the back end of a Pasteur pipette or a cut plastic transfer-pipette for the gentlest handling. Using tweezers, peel-of all adhering membranes and blood vessels. You can cut away the fimbriae with a scalpel or scissors. Be careful not to stab the hippocampi while proceeding.
4. Transfer cleaned hippocampi to a fresh dish of cold HBSS/20% FBS. Unroll the dentate gyrus. Use the subiculum to pin down the tissue while unrolling and cutting off the dentate gyrus with scissors. Also cut away the hippocampi from the remaining subiculum then transfer dissected hippocampi to a third 60 mm dish containing cold HBSS/20% FCS.
5. Cut hippocampi end to end, into about ten thin slices and store on ice until all dissections are completed.
6. Using a plastic pipette, under sterile conditions, transfer the sliced hippocampal tissue to a 15 ml polypropylene (not polycarbonate) conical tube. Let tissue settle and discard supernatant.
7. Wash 1× with 10 ml of HBSS/20% FBS, and 3× with 10 ml of HBSS alone.
   Digestion
8. Add one aliquot (10 mg) of trypsin and one aliquot of DNase (1 mg) to 2 ml of digestion solution and sterile filter directly onto the last pellet. Incubate for 5 minutes at 37° C., occasionally shaking gently. Discard solution and stop digestion by adding about 10 ml of HBSS/20% FBS.
9. Wash 1× with 10 ml of HBSS/20% FBS, and 1× with 10 ml of HBSS alone.
   Dissociation
10. Add one aliquot of DNase (1 mg) to 2 ml of dissociation solution and sterile filter onto last pellet as above. Fire-polish two sterile, siliconized/plugged Pasteur pipettes, making bore size of tips successively smaller. Too small tips will lyse the cells, too large tips will not dissociate cell aggregates. Mechanically dissociate cells by gently triturating, avoid bubbles, and always pipette onto the wall of the tube. Allow clumps of undissociated tissue to settle for 2 minutes then transfer supernatant to another tube. This separates cells from tissue particles.
11. Add 3 ml HBSS/20% FBS to the cell suspension (5 ml total), and centrifuge for 10 minutes at 1,000 rpm, 4° C.
12. Discard supernatant and resuspend pellet in 1.8 ml of pre-equilibrated plating medium. Place a small drop (˜0.1 ml) on each coverslip, using all the cell suspension. Incubate for 1-2 hours to allow the cells to attach. The first short cell processes should be visible by that time. Add 2 ml of pre-equilibrated plating medium to each dish and incubate for 48 hours.
13. After 2 days change the plating medium to selection medium by replacing 1 ml of old medium with 1 ml of fresh pre-equilibrated feeding medium. For the first change of medium, you will need to add 6 μM ARA-C, twice the normal concentration. By replacing only half the medium the ARA-C concentration is diluted in half. All subsequent feedings are done with the addition of 3 μM ARA-C.
14. Feed cells three times a week. Try to replace 1 ml of the old medium with 1 ml of fresh pre-equilibrated medium. However, always keep the neurons covered with medium as they are extremely sensitive and will die if exposed even briefly.

The experiments were performed in mature (>DIV 15) (Renger et al., 2001) high density cultures (synaptic density>1.5 synapses/μm² of dendritic surface area). We used a culture medium containing 0.8 mM Mg²⁺ (Gibco, 51200-038) for control cultures and added various amounts of MgCl₂ to raise the concentration. For short-term (4-6 hours) and long-term (48 hours) reduction of activity or Ca²⁺ influx, the following drugs were added to the culture medium: 10 μM nimodipine (Tocris), 5 μM flunitrazepam (Sigma), 1 μM NBQX (Sigma), 5 and 20 μM DL-AP5 (Tocris), 100 nM TTX (Biotium), or 10 μm baclofen (Tocris). All experiments involving animals were approved by the Massachusetts Institute of Technology's Committee on Animal Care.

Electrophysiology. Dual whole cell perforated patch clamp recordings were made on two interconnected cultured hippocampal pyramidal neurons. Perforated patch pipettes were front-filled with a solution containing (in mM): CsOH, 127; D-gluconic acid, 127; CsCl, 4; HEPES, 10; NaCl, 8; EGTA, 0.4; pH was adjusted to 7.25 with CsOH, and then back-filled with the same solution containing 150-220 ng/ml amphotericin B (Sigma, St. Louis, Mo.). Extracellular solution contained (in mM): NaCl, 145; KCl, 3; glucose, 15; HEPES, 10; MgCl₂, 0.8-1.2; CaCl₂, 1.2; 0.005 glycine (Sigma), 0.05 picrotoxin (Sigma); pH adjusted to 7.4 with NaOH. MgCl₂ concentration was matched to its concentration in culture medium. In experiments where effects of drugs on neuronal activity were quantified (FIG. 4D), recordings were done in culture medium (with HEPES replacing bicarbonate/CO₂ to maintain pH). All experiments were performed at room temperature. For assaying synaptic connectivity, each neuron was stimulated by 1 ms step depolarization from −70 to +30 mV in voltage-clamp mode. Only neurons with monosynaptic connections were used. The access resistances of both pre- and postsynaptic neurons were monitored online and were typically 7-20 MΩ. Recordings with access resistance>20 MΩ or that varied substantially were rejected from analysis.

FM 1-43 Loading and Destaining. Functional presynaptic boutons were stained with 10 μM FM 1-43 (synaptogreen, Biotium) by eliciting 30 APs at 0.5-1 Hz. This loading protocol was chosen to avoid short-term plasticity and “re-use” of vesicles through “kiss-run” mode of exocytosis (Aravanis et al., 2003). The neurons were stimulated to fire action potentials by passing 1 ms 50 mA current through platinum electrodes placed at a distance of 7 mm from both sides of the chamber. To ensure reliable action potential initiation, the current amplitude was chosen to be 50% above the threshold for action potential generation, as confirmed by whole-cell patch clamp recording. To prevent recurrent activity, excitatory postsynaptic responses were blocked completely by the addition of DL-AP5 (50 μM, Sigma) and NBQX (10 μM, Sigma) during FM loading and unloading procedures. FM dye was present when terminals were stimulated and 30 s after the stimulation (Ryan et al., 1993). For single action potential loading, the duration of dye exposure was reduced to 15 s to minimize nonspecific staining. Following this loading protocol, any external dye that had not been taken up into terminals was washed away in Ca²⁺-free solution with the addition of quencher ADVASEP-7 (100 μM, Sigma) to speed up dye removal from external membranes (Kay et al., 1999; Zakharenko et al., 2001). Unloading was induced by 2 Hz stimulation for 4 minutes.

Imaging and Image Analysis. Imaging was performed using an Olympus (FV300) confocal laser inverted microscope. The 488 m line of the argon laser was used for excitation, and the emitted light was filtered using a 510 nm long pass filter and detected by photomultiplier. A 40×1.15 NA water-immersion objective was used for imaging. For experiments including one AP loading (FIG. 1, Supplemental materials), images were collected at a resolution of 1024×1024 with a pixel width of 0.11 μm. Confocal aperture was set to maximal. Each image was the average of four images separated by 0.8 μm steps in the z-direction. For all other experiments, a confocal aperture was partially open and image resolution was reduced to 0.138 μm/pixel. The gain of the photomultiplier was adjusted to maximize the signal/noise ratio without causing the saturation by the strongest signals. The image after FM dye unloading was subtracted from the initial image; thus only those terminals containing activity-dependent releasable FM dye (˜90% of total staining) were analyzed. FM positive puncta were selected for further analysis using custom scripts written in ImagePro Plus (Media Cybernetics, Carlsbad, Calif.) and MATLAB (Mathworks, Natick, Mass.) programs based on following criteria: the fluorescence intensity (ΔF) was 3 standard deviations above the mean background and the diameter of spots was between 0.1-0.6 μm.

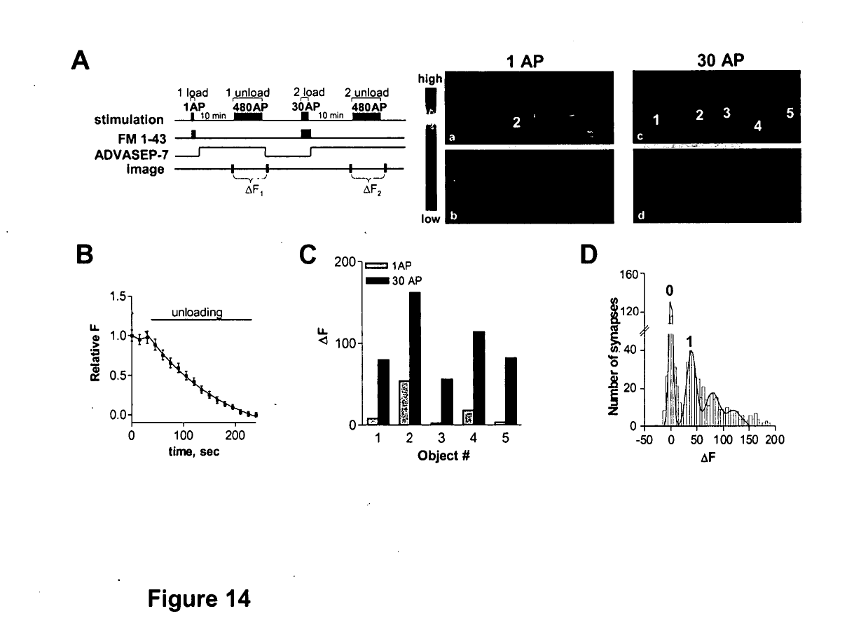

Determination of fluorescence of a single vesicle (F_{Q}). We have followed previously developed approaches to determine the value of F_{Q} (Ryan et al., 1997; Murthy and Stevens, 1998; Aravanis et al., 2003). Essentially, it is assumed that the number of vesicles exocytosed following a single AP should be small, in the range of one or two vesicles. Therefore, ΔF_{#AP=1} (where AP=1 symbolizes that a single action potential was employed) should quantify the distribution of FM dye for a small number of synaptic vesicles. FIG. 14A displays the FM dye puncta following a single AP loading procedure. Comparing it with the FM dye image obtained after 30 AP loading procedure, we find that fewer synaptic terminals were stained with FM dye in the former case, and for those terminals that did take up FM dye, ΔF_{#AP=1} was significantly smaller than ΔF_{#AP=30}. On average, there is a 5.7 (±1.3)-fold difference between ΔF of the same boutons loaded with 1 or 30 AP (FIG. 14C), suggesting a correlation between ΔF and the number of action potentials applied. To determine the value of F_{Q}, we identified the locations of functional synapses by the 30 AP-loaded image, and using this, obtained and plotted the intensity distribution of ΔF_{#AP=1} (FIG. 13D, 841 boutons). The F_{Q} was calculated by fitting the intensity of ΔF after single AP loading with the sum of multiple Gaussinas (Aravanis et al., 2003):

$$T ( VF ) = {\sum }_{k = 0}^{3} {v}_{k} {ⅇ}^{- \frac{{( VF - {kF}_{Q} )}^{2}}{2 ( {σ}_{0}^{2} + k {σ}_{v}^{2} )}} \qquad ( 1 )$$

where T is the number of events at a given ΔF, vₖ is the amplitude of the kth peak, σ₀² is the variance of measurement error, σᵥ² is the variance in fluorescence of a single FM1-43 labeled vesicle and was set to be 0.2*F_{Q} (Schikorski and Stevens, 1997). Thus, histograms were fitted by Eqn. 1 with free parameters F_{Q} and v_{k(0 to 3)}.

The distribution of ΔF_{#AP=1} has discrete peaks, reflecting the quantal nature of fluorescent membrane dye staining, while the variance in each peak was assumed to originate from the noise of measurement and natural variation in vesicle size (Ryan et al., 1997; Murthy and Stevens, 1998; Aravanis et al., 2003). The 0^{th} peak, centered at the origin of the plot, represents functional synapses that failed to take up FM 1-43 while the first peak corresponds to boutons loaded with a single vesicle. Because of the lower number of events in the second and the third peaks, their peak locations could not be determined conclusively. We determined the F_{Q} by fitting the histogram to the sum of four gaussian functions with equal peak spacing and found F_{Q} to be 39.8 fluorescence units. The average F_{Q} value from 4 experiments was found to be 41.2±1). Several additional tests were performed to confirm that the value of F_{Q} determined by this approach was proportional to the amount of FM dye releasable from a single vesicle. We found that the F_{Q} value was sensitive to dye concentration: it increased by 1.4-fold when FM 1-43 concentration was increased from 10 to 15 μM (see also Murthy and Stevens, 1998). Furthermore, F_{Q} remained similar in experiments where only miniature release was measured (data not shown) (Ryan et al., 1997). Determination of F_{Q} permits us to monitor terminals with extremely low Pr. For example, applying 30 APs under such conditions will ensure the detection of functional terminals with Pr>0.04. Although increasing the number of action potentials would increase the sensitivity of detection of low Pr synapses, it would limit the upper bound of the dynamic range for detecting an activity-induced up-regulation of Pr.

Statistical analysis. Error bars shown represent the standard error of the mean (SEM). Statistical significance was considered to be p<0.05 and is indicated in the figures by an asterisk. For each experimental condition that contributed to the analysis, N represents the number of separate cultures used and n represents the total number of individual synapses. Means were compared with the t-test.

Results

To study how the plasticity of synapses is regulated within neural networks we obtained information on the physiological properties of individual synapses before the induction of synaptic plasticity. This information can help to determine what is necessary for a synapse to be plastic. In order to gather such information we first checked the release probability (Pr) of single CNS synapses by measuring presynaptic vesicle turnover using activity-dependent FM dyes (Ryan 1996; Murthy 1997; Zakharenko 2001).

A fixed number of action potentials was delivered by field stumulation at low frequency (0.5-1 Hz), which in turn triggered turnover of presynaptic vesicles. Exocytosed vesicles were labeled by reuptake of the dye FM 1-43 that was present in the bath. FIG. 1B (upper panel) illustrates a typical image obtained after FM 1-43 loading. Labeled vesicles are clearly visible. The column at the right of the figure indicates intensity of staining. To confirm that these FM dye spots indeed correspond to functional synaptic terminals, we applied another set of action potentials to trigger exocytosis. If the FM dye is stored in releasable synaptic terminals, exocytosis triggered by the second set of action potentials woud be expected to result in release of FM dye and destaining of these loaded terminals. As shown in FIG. 1B (lower panel), the second set of action potentials caused complete destaining of the synaptic terminals. Similar to previous reports, the amount of dye loaded is proportional to the number of action potentials and the concentration of Ca2+ in extrajunctional solution (data not shown), indicating that the degree of FM dye loading in individual synaptic terminals is positively correlated with the Pr of synaptic terminals. These results confirm the utility of FM 1-43 dye staining to measure presynpatic function.

### Example 2

### Identification of Functionally Silent Presynaptic Terminals in Neural Networks

Materials and Methods

Culture of hippocampal neurons, electrophysiology, and dye loading, synapsin staining, and image analysis were performed as above except that AM 1-43, a new fixable variant of FM 1-43 was used. For dye loading, 30 action potentials (AP) were applied at 1 AP/sec.

Immunohistochemistry. Following functional FM 1-43 staining, neurons were fixed by flooding the perfusion chamber with a fixative FSB solution consisting of 4% paraformaldehyde and 4% sucrose in 1×PBS for 30 minutes and permeabilized with 0.5% Triton X-100. Primary antibodies against VGLUT1 (Chemicon International), and GAD-65 (Chemicon International) were applied for 8 hours, followed by rinses in PBS and staining with Alexa 488- and 633-conjugated secondary antibodies ( 1/400; Molecular Probes, Eugene, Oreg.) at 22-24° C. All images were collected at 1024×1024 pixels with a 0.069 μm/pixel resolution. 11 images separated by 0.8 μm steps in the z-direction were compressed to generate the final image. Images of the fixed and immunolabeled tissue were aligned with corresponding FM images of the same region. Synapsin staining was performed essentially as described (Renger 2001).

Results

The approach described in Example 1 offers the opportunity to simultaneously determine the functional status of large numbers of synaptic terminals. This allows us to compare the Pr of synaptic terminals within networks under different conditions. The first question we asked was whether all synaptic terminals have a sufficiently high Pr to be detected by this approach. To answer this question, we needed to determine whether all synaptic terminals are stained by this protocol. This can be determined using a new fixable variant of FM 1-43 (AM1-43) to label functional synaptic terminals, followed by fixing neurons and labeling their synaptic terminals with an antibody to the presynaptic vesicle-associated protein synapsin I. If all the presynaptic terminals were functional, the number of FM positive terminals should be close to the number of synapsin I-positive terminals. FIG. 1C shows AM dye staining, synapsin staining, and a merged image. Surprisingly, 70% of presynaptic terminals were not stained by this stimulation protocol (FIG. 1D). We have checked the sensitivity of our confocal image system and found that the system is capable of detecting the uploading of AM dye into a single synaptic vesicle. Since 30 action potentials were applied during AM-dye loading period, terminals with Pr>0.04 should be detectable by our image system. Thus, these data suggest that 70% of synaptic terminals have a Pr of less than 0.04, and are therefore functionally silent.

To confirm that these silent terminals have very low Pr, we stimulated synaptic terminals maximally with 90 mM K solution for 1 minute in the presence of AM dye. This stimulation generates prolonged opening of Ca channels and is capable of activating all synaptic terminals, as has been demonstrated previously [Betz & Bewick 1992; Ryan 1993]. Under this condition of complete exocytosis, the great majority of synaptic terminals detected by synapsin I antibody were also stained by AM dye, suggesting that terminals that fail to take up dye with the 30 action potential stimulation protocol indeed have a very low Pr. FIG. 1D shows the density of synapses that showed activity in response to applied action potentials (AP) compared with the density of synapses identified structurally using staining for synapsin I (structural) and the density of synapses the density of synapses that showed activity in response to stimulation with high K⁺. These data indicate that under typical action potential stimulation protocols the majority of presynaptic terminals do not release neurotransmitter, i.e, they are silent.

To further determine the number of these very low Pr terminals and their proportion to the whole population of functional synaptic terminals, we compared the co-localization of FM puncta obtained for the 30 AP loading with puncta obtained using fluorescent antibodies against specific presynaptic proteins. VGLUT1 and GAD-65 antibodies were used to mark glutamatergic and GABAergic terminals, respectively. After FM images were obtained, the specimens were rapidly fixed and stained with antibodies. The two sets of images were aligned, allowing comparisons of functional terminals with their structural equivalents. Most FM puncta were co-localized with spots labeled by antibodies against presynaptic proteins (Ryan et al., 1993; Schikorski and Stevens, 2001). To ensure that these functional presynaptic boutons were part of functional synapses, composed of both pre- and post-synaptic machinery, we examined whether spots labeled by VGLUT1 antibody were co-localized with those marked by PSD-95 antibody, a critical protein presented in postsynaptic spine of glutamatergic synapses. All VGLUT1 spots were co-localized with PSD-95 spots (data not shown). Thus, most of FM dye uptake occurred specifically within structurally complete synaptic terminals. However, not all synaptic terminals took up FM dye after 30 APs (FIG. 1E). In fact, only 46% of structural synapses were stained during the 30 AP stimulation/loading protocol (data not shown). To determine the functional status of these seemingly non-functional terminals, we increased the technique's sensitivity by increasing the number of APs to 150 and the frequency of stimulation to 5 Hz. This protocol facilitates the release of vesicles from low Pr synapses without extensive depression of high Pr synapses (Murthy et al., 1997). Twenty percent more functional synapses were detected by this procedure, also consistent with a previous report (Murthy et al., 1997). To determine whether the remaining 30% of synapses were capable of having vesicle turnover at all, we applied a maximal stimulation protocol (600 AP@ 20 Hz). Compared with labelling under the 30 AP protocol in the same region, the number of FM positive puncta increased to 88% of structurally identified synapses.

We have interpreted the lack of FM uptake after 30 action potentials as meaning that the terminals have too low a Pr to exocytose their vesicles even after such a sequence of depolarizing events. An alternative possibility is a potential failure of action potentials to reach synaptic terminals. We considered this unlikely for the following reasons: (i). Synaptic failure in hippocampal slices could not be attributed to the failure of action potential propagation to synaptic terminals (Allen and Stevens, 1994). (ii) It has been demonstrated previously that Ca²⁺ flux in synaptic terminals is reliably generated by field stimulation under identical configuration conditions (Ryan and Smith, 1995). (iii). The numbers of FM positive puncta detected after maximal electrical stimulation (600 AP @ 20 Hz) were similar to those obtained by 50 mM K⁺ stimulation, a method that depolarizes all terminals directly by generating prolonged opening of Ca²⁺ channels (Betz et al., 1992; Ryan et al., 1993). (iv). In the presence of 100 ng/ml of neurotrophin BDNF, which is known to increase the Pr of synaptic terminals without changing their electrical excitability (Li et al., 1998), we found that all structural synaptic terminals became capable of taking up FM dye with the 30 AP protocol (data not shown). In summary, our results show that most synapses had a relatively low Pr (median=0.14), and that roughly fifty percent of all structural synapses had a Pr<0.04.

### Example 3

### Inability to Convert Silent Synaptic Terminals to Functional Terminals with Theta-Burst Stimulation

Materials and Methods

These were generally as described in Example 1. For theta-burst stimulation, each burst contains 5 action potentials with 40 ms interval. 30 bursts were delivered, the burst interval was 500 ms.

Results

The presence of silent synaptic terminals within neural networks provides a great potential to up-regulate the strength of their synaptic connections rapidly. Thus, we are interested in whether these silent synaptic terminals can be converted to functional ones by patterns of synaptic activity. To test this possibility, we checked whether the up-regulation of the Pr of synaptic terminals could be induced by theta-burst stimulation (TBS), a plasticity induction method that is believed to replicate neural activity in vivo (Bliss and Collingridge, 1993). The induction-associated changes in Pr of synaptic terminals were determined by comparing F₁ and F₂, before and 30 minutes after TBS (30 bursts, each burst contains 5 APs @ 25 Hz, 500 ms inter-burst interval). Using the experimental protocol described in FIG. 2A, the changes in Pr of synaptic terminals was determined by comparing F₁ and F₂ before and 30 min after theta-burst stimulation. F₁ quantitavely represents fluorescence at individual synapses. Its value is proportional to Pr of a synaptic terminal before any stimulation. F₂ represents fluorescence at the same synapse after stimulation. Functional presynaptic terminals would be expected to take up the dye and thus become fluorescent. Surprisingly, no significant change in FM dye loading, and thus no Pr up-regulation, was observed following the theta-burst induction protocol, as shown in the representative image (FIG. 2B). No changes were found in the distribution of release probabilities (FIG. 2C) or individual synapse Pr (FIG. 2D). On average, the F₁/F₂ (ratio change of fluorescence per FM spot) was 0.9±0.2 and N₂/N₁ (ratio of number of FM positive terminals) was 1.2±0.2. There were also no significant differences in S₂/S₁ (1.1±0.4, n=8432, N+13, p=0.15). In FIG. 2B, each circle represent a synapse, with F₂ representing the fluorescence of that synapse at a time before stimulation and F₂ representing the fluorescence of the same synapse after stimulation and another exposure to the fluorescent dye. Therefore, if the theta-burst stimulation protocol had induced the silent synapses to become active, most of the circles would be expected to lie above the 45 degree line. One of ordinary skill in the art will appreciate that either F₁ and F₂ or ΔF₁ and ΔF₂ could be used.

We used F*N (i.e., the product of F and N) to represent the total synaptic strength (S) within a given region of neural networks. There were no significant differences in the ratio of S₂/S₁ (1.1±0.4,N=8, p=0.15). Thus application of a theta-burst protocol that mimics the activity normally seen in the hippocampus was unable to convert functionally silent synapses into active synapses. Similar results were observed 10 min following the induction protocol, suggesting a lack of short-term plasticity (N=4, p=0.3). To test whether the applied induction protocol is sufficient to reach LTP threshold [Bear 2003], we increased the total number of action potentials to 600. Still, there were no detectable changes of synaptic strength (S₂/S=1.2±0.4, N=4). These data suggest that functionally silent synaptic terminals are not by default potentiable. FIG. 15 presents results of additional experiments suggesting that reduction of neural activity induces enhancement of synaptic plasticity.

### Example 4

### Reduction of Neuronal Activity Triggers Formation of a Highly Plastic Network

Materials and Methods

These were as described in the preceding examples except that cultures were pretreated with 50 nM TTX for 6 hours prior to dye staining and application of theta-burst stimulation.

Results

To test whether the reduction of neural activity can enhance synaptic plasticity, we pretreated cultured neurons with a low concentration of the Na⁺ channel blocker TTX (50 nM) to reduce, but not abolish, network activity. This agent is well known to block inhibit the development of action potentials. At this concentration, spontaneous neural network activity remains, but the average frequency of action potentials is reduced relative to control levels. Since long-term reduction of neural activity (>24 hour) can also trigger homeostatic regulation of synaptic strength [Turrigiano 1998; Murthy 2001], we restricted TTX treatment to 6 hours to avoid inducing this effect. We then tested the plasticity of presynaptic terminals in the TTX treated culture using the same protocol we applied to control cultures (FIG. 2A). Strikingly, FM dye loading increased significantly after theta-burst stimulation (FIG. 3A).

Quantitatively, there are two distinct changes in FM 1-43 staining properties: (i) an increase in the fluorescence intensity of individual puncta which is detectable during the first dye loading process, suggesting an increase of Pr at these terminals; and (ii) an increase in the number of FM positive boutons, indicating a conversion of non-functional terminals to functional ones (FIG. 3B). For the experiment depicted in FIG. 3A, the median value of F increased two-fold, while the number of detectable fluorescent spots increased from 250 to 1150.

Results in previous studies indicate that the degree of synaptic plasticity is inversely correlated with their initial synaptic strength [Bi & Poo 1998; Montgomery 2001). To check whether this relationship holds in this experimental condition, we plotted the change of Pr (F₂/F₁) as a function of the initial Pr (F₁). Clearly, the magnitude of synaptic plasticity in individual terminals is inversely correlated with their initial Pr (FIG. 3C). Thus, the lower the initial Pr of an individual synapse, the higher the synapse's chance of potentiation. Average results of these experiments (6 experiments, 1715 synapses) are shown in FIG. 3D. After theta-burst stimulation, probability of release increased by 1.8-fold (p<0.001, N=6), accompanied with a 3-fold increase in FM-positive synaptic density (p<0.001, N=6). Total presynaptic strength (F*N) increased 5.4-fold in TTX-treated cultures (black bars).

Since the activation of NMDA receptors during theta-burst stimulation is essential for the induction of synaptic plasticity, we tested whether the increase of Pr also requires NMDA receptor activation. No detectable changes of Pr were observed in the presence of the NMDA receptor blocker D-AP5 (50 mM) during theta-burst delivery (gray bars, N=5, p=0.2), indicating the necessity of NMDA receptor activation to induce this type of synaptic plasticity. These data indicate that an overall reduction in the level of activity of a neural network over this time period resulted in increased plasticity of the network, i.e., increased ability of synapses in the network to respond to a pattern of stimulation by increasing their strength.

### Example 5

### Modification of Neural Activity Leads to Increased Ca⁺⁺ Induced Upregulation of AMPA Receptors in Postsynaptic Spines

Materials and Methods

These were generally as described in the preceding examples. Glutamatergic iontophoresis was perfomed as described [Murnick 2000].

Results

Although the above protocol allows us to rapidly determine a before and after picture of the status of a large number of individual pre-synaptic terminals, it fails to tell us their postsynaptic properties or the second-by-second dynamics of plasticity and does not provide information on functional electrophysiological changes. Since we wished to correlate the above pre-synaptic findings to the plasticity of single or small clusters of postsynaptic locations, this ruled out traditional double-patch plasticity experiments in which heterogeneous populations of connections are studied. Such studies also usually confound pre-synaptic and postsynaptic effects. For this purpose, we developed a technique of high-temporal and spatial resolution glutamatergic iontophoresis that allows us to stimulate single or small clusters of postsynaptic locations (resolution ˜2 μm, 1 ms; [Murnick 2000]).

To determine whether a decrease in overall activity of a neural network leads to increased plasticity (i.e., an increase in synaptic strength in response to a pattern of stimulation) of postsynaptic locations, we assessed the effect of treatment with Mg⁺⁺ on the ability of postsynaptic locations to respond to glutamate. As described above, Mg⁺⁺ blocks the NMDA receptor channel at resting membrane potential, so Mg⁺⁺ treatment reduces the overall activity level of the neural network. As also described above, increases in postsynaptic intracellular Ca⁺⁺ stimulated by activation of NMDA receptors leads to recruitment of AMPA receptors and their insertion into the postsynaptic membrane. This insertion leads to increased responsiveness to subsequent stimulation with glutamate. Under control conditions (0.8 mM Mg⁺⁺ treated cultures), postsynaptic evoked current responses (0.2 Hz; voltage clamp at −70 mV) showed stable responses for the duration of recording (30 minutes recording shown in FIG. 4A). On the other hand, functional networks that had been treated with increased Mg⁺⁺ to reduce levels of neural activity (1.2 mM Mg⁺⁺ chronic treatment), showed significant levels of AMPA-R insertion even with the minimal levels of Ca⁺⁺ flux observed at −70 mV, as indicated by the increases in current seen following application of glutamate (FIG. 4B). This remarkable effect was due to the small Ca⁺⁺ influx generated per synaptic response, as the NMDA antagonist D-AP5 blocked it, and subsequent D-AP5 washout released it (FIG. 4C).

### Example 6

### Mg⁺⁺ and GABA_{B} Agonists Increase Synaptic Plasticiy on a Long Term Basis; Various Other Agents that Reduce Ca⁺⁺ Flux Increase Synaptic Plasticity on a Short Term Basis

We sought to assess the ability of various treatments to increase synaptic plasticity. In functional neural networks, the rate of neural firing is largely dependent on the functional balance of excitation and inhibition [Kandel, supra] Thus, shifting this balance will perturb the overall level of neuronal firing. We used three approaches to reduce activity. We reduced the strength of excitatory synapses by either (i) enhancing presynaptic inhibition of glutamatergic synapses with a GABA_{B} receptor agonist (baclofen, 20 μm) or (ii) blocking AMPA receptor activation directy with an AMPA receptor antagonist (NBQX, 1 μm). We also reduced overall neural activity by enhancing inhibitory synaptic transmission by prolonging the duration of endogenous GABA_{A} channel openings with an allosteric agonist acting on the GABA_{A} receptor (flunitrazepam, 5 μm) or by blocking calcium channels (nimodipine, 10 mM). These treatments allowed us to identify new potential regulation sites of synaptic plasticity and identify agents that enhance synaptic plasticity. All these treatments led to a reduction of neural activity in networks (data not shown).

Again, cultured neurons were treated for 4-6 hours before the experiments, to be comparable with the TTX treatment described above. Following treatment, cultures were stimulated with a theta-burst stimulation protocol. FIG. 5A shows an example of an FM image of several thousand synapses in baclofen treated cultures before and after theta-burst stimulation. Both the number of functional synapses and the probability of release increased after theta-burst induction in baclofen treated cultures. Therefore, the reduction of glutamate release for four hours, and corresponding reduction in overall activity of the neural network, results in formation of a highly plastic network. Similar results were found for NBQX and flunitrazepam treated cultures (FIG. 5C). In all treatments substantially more synapses became FM positive after theta-burst stimulation in the cultures that had been treated than in the control cultures (N2/N1, 2-3 fold increase). There was also a moderate increase in the average fluorescent intensity change (F₁/F₂, 1.5 fold increase) in the treated versus control cultures and a significant difference in overall presynaptic strength (S₂/S₁), with the treated cultures showing 2.5-4.5 fold greater strength than the untreated culture (FIG. 5C). Thus, reduction in the strength of excitatory synaptic connections and an increase in the strength of inhibitory synaptic connections were each effective in enhancing the plasticity of presynaptic terminals, suggesting the importance of balancing excitation and inhibition in the maintenance and induction of synaptic plasticity.

We hypothesized that one possible candidate by which intracellular regulatory mechanisms might detect the level of neural activity is Ca⁺⁺ flux though NMDA receptors and voltage-gated Ca⁺⁺ channels (e.g., L type Ca⁺⁺ channels). The opening of both channels depends strongly on the neural activity levels. Thus, we tested whether a reduction of Ca⁺⁺ flux is sufficient to enhance the plasticity of presynaptic terminals. As mentioned above, Mg⁺⁺ blocks NMDA receptors in a voltage-dependent manner, and the degree of voltage-dependent blockade depends on its concentration in the extracellular solution [Mayer 1984; Nowak 1984; Jahr & Stevens 1990]. Thus, by increasing the extracellular Mg⁺⁺ concentration, we can increase the voltage-dependence of the Mg⁺⁺ block. Under these conditions, the Ca⁺⁺ flux in resting and sub-threshold conditions will be reduced, while Ca⁺⁺ flux triggered by bursting will not be affected, as large depolarization during bursts is sufficient to remove the Mg⁺⁺ block. For similar reasons, we altered Zn⁺⁺ concentration to test its efficacy in enhancing synaptic plasticity. As a comparison, we also tested the effect of the L-type Ca channel blocker nimodipine. FIG. 5D shows that the effects of partial block of Ca flux through addition of 0.4 mM Mg⁺⁺ (to 0.8 mM in control cultures bringing the total concentration to 1.2 mM, N=12), 5 mM Zn⁺⁺ (N=6), or 10 mM nimodipine (N=3) on the plasticity of presynaptic terminals. Two weeks of treatment with Mg⁺⁺ and Zn⁺⁺ resulted in significant increases of plasticity in presynaptic terminals, while treatment with nimodipine for 2 days appeared to result in a more modest effect. Additional experiments indicated that the actual effect of nimodipine after 2 days was lower and that it did not persist after longer time periods. Since none of these treatments induces a significant change in average firing rates of neurons within cultured neural networks, we conclude that Ca⁺⁺ influx is a factor of major importance in determining the plasticity of presynaptic terminals.

To check if these treatments changed the structural density of synapses, we used immunostaining to label and count excitatory and inhibitory terminals. An antibody to vesicular glutamate transporter VGLUT1 was used to label glutamatergic synapses, while an antibody to GAD-65, an enzyme in the biosynthetic pathway for the inhibitory neurotransmitter GABA, was used to label their inhibitory equivalents. No short treatment (2-24 hr, baclofen, nimodepine, NBQX, and TTX) produced a significant change of synaptic density. On the other hand, long term increases (two weeks) of Mg⁺⁺ concentration in culture medium led to a moderate increase of synaptic density by ˜30% (N=4, p=0.001).

Since we found that a transient reduction of neuronal activity for 4-6 hours by treatment with TX, NBQX, flunitrazapan, or nimodipine increases the plasticity of the network, we asked whether a long-lasting reduction of neuronal activity, over a period of days, would generate similar results as was the case for treatment with divalent cations. To answer this question, we compared the degree of synaptic plasticity after 4 and 48 hours of drug treatment. Surprisingly, after 48 hours of incubation there was no significant TBS-induced potentiation whether the drug inhibited global activity (TTX, NBQX, flunitrazepam, (FIG. 5E) or decreased Ca²⁺ flux (AP-5, nimodipine, FIG. 5F). In both cases, the neurons appeared healthy. One possibility is that due to homeostatic mechanisms (rev. Turrigiano and Nelson, 2004) the chronic inactivation led to a saturating increase of presynaptic efficacy (Bacci et al., 2001; Murthy et al., 2001; Thiagarajan et al., 2002) which in itself made them incapable of being further potentiated. Indeed we found that the Pr is increased by 50% after 48 hours of activity blockade (FIG. 5G). However, the TBS-induced potentiation in the short-term treated cultures was of 400% (FIG. 5G). Despite the modest increase of Pr, these synapses had ample dynamic range to be potentiated. We can infer that the lack of plasticity during long-term reduction of Ca²⁺ flux is likely to be caused by other mechanisms.

To further explore our finding that elevation of [Mg²⁺]ₒ triggers formation of highly plastic synapses, we cultured hippocampal neurons with both “normal” and “elevated” concentrations of [Mg²⁺]ₒ (control: 0.8 mM—experimental: 1.2 mM; 2 weeks). Neurons growing under the higher level of [Mg²⁺]ₒ had normal neuronal density and morphology. Unlike the control neurons, however, their synapses were highly plastic even after the long-term treatment. FIG. 6A provides representative images of presynaptic terminals before and 30 minutes after TBS in high Mg²⁺ treated cultures. There are dramatic, sustained increases in FM 1-43 staining after TBS stimulation. The Pr increased 2.3-fold (±0.1, p<0.001), and the number of detectable FM puncta increased 2.1-fold (±0.1, p<0.001), resulting in a 5.5-fold increase in total presynaptic strength (n=10618, N=15, p<0.0001, FIG. 6B). This potentiation was stable for at least 1.5 hours (data not shown). This form of plasticity required NMDAR activation (50 μM AP-5 blocked induction; S₂/S₁=1.1±0.2, n=3691, N=5). In contrast to uniform reductions of Ca²⁺ flux, synapses retained their plasticity in elevated concentrations of Mg²⁺.

We attributed this effect to the ability of Mg²⁺ to selectively influence Ca²⁺ flux associated with uncorrelated activity. However, the lack of long-lasting enhancement of plasticity by AP5-mediated blocking of NMDA channels may have been due to the latter's complete blockade of NMDA-mediated Ca²⁺ flux. To determine whether this is the case, we repeated the experiment with a concentration of AP-5 (5 μM; ˜IC₅₀) that matched the degree of Ca²⁺ flux reduction as that obtained via an increase in [Mg²⁺]ₒ. The magnitude of presynaptic plasticity was tested in cultures following 4, 48 hours, 2 and 3 weeks of incubation with either 5 μM AP-5, 20 μM AP-5 or 1.2 mM [Mg²⁺]ₒ. The response to 4-hour reduction in Ca²⁺ flux was very similar across all treatments. The magnitude of enhancement depended only on the degree of blockade. Complete blockade of NMDA receptor or L-type Ca²⁺ channels induced a ˜4 fold enhancement of synaptic plasticity (FIGS. 5F and 6C). Reduction of NMDAR activity by 50%, whether through application of 5 μM AP-5 or an increase in [Mg]ₒ (0.8 to 1.2 mM), resulted in a ˜2 fold enhancement of synaptic plasticity (FIG. 6C). On the other hand, for long term treatments, only the synapses grown under elevated [Mg]ₒ retain high plasticity persistently. Thus, it was not the amount of blockade that determined the duration of plasticity enhancement. Rather, it is the blockade that preferentially reduced the Ca²⁺ influx during periods of uncorrelated activity that generated a long-term increase of plasticity.

To test if the Mg²⁺-induced effect was reversible, the cultures were incubated for 2 days in 1.2 mM [Mg²⁺]ₒ and then switched back to control conditions (0.8 mM [Mg²⁺]ₒ). Exposure to increased [Mg²⁺]ₒ for 2 days induced a 3.9-fold increase in total presynaptic strength after induction (n=2234, N=3, p<0.001; FIG. 6D). The terminals in higher Mg²⁺ lost their plasticity and returned to control conditions 48 hours after being switched back to 0.8 mM [Mg²⁺]ₒ (S₂/S₁ is 1.1±0.2, n=2739, N=3, p>0.2). These data suggest that perturbations in Ca²⁺ flux can reversibly regulate the plasticity of synaptic terminals.

To confirm that such a prominent TBS-induced increase in Pr does contribute to the enhancement of total synaptic strength, the magnitude of potentiation was tested electrophysiologically by sampling field EPSCs (fEPSCs) before and after TBS. The whole cell recording was obtained under perforated patch configuration to avoid wash-out of the intracellular components critical for synaptic plasticity. The membrane potential was clamped at −70 mV and EPSCs were evoked by field stimulation at a sampling frequency of 0.03 Hz. [Mg²⁺]ₒ during sampling was 4 mM to reduce background neural activity. The intensity of field stimulation was adjusted to produce a half maximal fEPSC amplitude. After 30 minutes of sampling were employed to determine the baseline level of synaptic strength, the bath solution was changed to 1.2 mM [Mg²⁺]ₒ and the recording configuration to current-clamp mode to allow for membrane voltage fluctuations. TBS was delivered at the same amplitude and temporal profiles as for the FM experiments. In control cultures (FIG. 6E-F), as expected, fEPSC size did not change significantly after TBS (0.9-fold change, p>0.2). In contrast, in the Mg-treated cultures, TBS induced a 2.7-fold increase in fEPSC amplitude (p<0.0001, FIG. 6E-F). The increase in fEPSC reached a maximum ˜10 minutes after TBS and lasted at least 1.5 hours. On average, no significant change in synaptic strength was found in control cultures (1.0±0.1, N=4); but synaptic strength increased 2.7-fold (±0.6, N=5) after TBS induction in the elevated Mg²⁺ treatment cultures (FIG. 6G).

Given the important role that neural activity plays in the functionality of neural networks, we suspect that synaptic plasticity might not be highest when neural activity is blocked completely. To determine whether this is the case we measured presynpatic strength, as assessed by FM dye uptake, in cultures that were treated with various concentrations of Mg⁺⁺, Zn⁺⁺, or TTX to alter overall activity of the neural network. As shown in FIG. 6H, there is a “bell”-shaped dependence of plasticity on extracellular Mg⁺⁺ concentration with an “optimal” concentration at 1.2 mM. Similar results were obtained in zinc-and TTX-treated cultures; chronic (2 weeks) inhibition of background Ca⁺⁺ influx by 2 mM of Zn⁺⁺ (FIG. 6I) or 2-days incubation with 50 nM TTX (FIG. 6J) produced a maximal level of potentiability, whereas concentrations higher or lower than this compromised the plasticity of the network. These experiments suggest that there is an optimal level of network activity that maintains the plasticity of neural circuitry.

### Example 7

### Temporal Patterns of NMDA Mediated Ca²⁺ Flux Can be Modified by [Mg²⁺]ₒ

Materials and Methods

Analysis of voltage-dependence of NMDAR Mg²⁺ block. For studying the voltage-dependence of NMDAR Mg²⁺ block, NMDA currents were evoked by local application of glutamate using the high-speed iontophoresis technique (Murnick et al., 2002). The current traces for each cell were normalized to their value at +40 mV membrane potential. The voltage dependency of NMDA currents was modeled using the Woodhull function (Woodhull, 1973). The NMDA channel conductance in the presence and absence of Mg²⁺ was related according to the relationship:

$$\frac{g}{{g}_{max}} = \frac{1}{1 + \frac{{[ {Mg}^{++} ]}_{o}}{{K}_{Mg}} {ⅇ}^{( - \frac{z δ EF}{RT} )}} \qquad ( 1 )$$

where K_{Mg} is the voltage-independent affinity of Mg²⁺ for the channel (0 mV membrane potential), δ is the electrical distance of the Mg²⁺ binding site in the membrane field, E is membrane potential, and z is the valence of the blocking ion. RT/F was 25.4 mV (21° C.).

Calcium imaging. Olympus (FV300) confocal laser-scanning system was used to perform calcium and structural imaging. The single wavelength calcium indicator Fluo-5F (K_{d}˜1.6 μM, Molecular Probes) was loaded into the neuron under a whole cell patch clamping configuration. The intracellular pipette solution contained (in mM): 130 CsMeSO₃, 10 Hepes, 10 Sodium phosphocreatine, 4 MgCl₂, 4 Na₂-ATP, 0.4 Na₂-GTP, 0.02 Alexa Fluor-633 (Molecular probes); pH was adjusted to 7.25. The pipette resistance ranged from 2-3 MΩ and calcium imaging was performed at room temperature. For most experiments, images were taken 30-40 minutes after establishing whole cell recording to allow equilibration of indicators in spines located 50-150 μm from the cell body (where the actual measurements were performed). NMDA receptors at single spine were activated by local application of glutamate using high-speed iontophoresis technique every 20 sec. Fluo-5F and Alexa Fluor-633 were excited with two different wavelengths, 488 and 633 nm, by argon and helium lasers, respectively. Imaging with Alexa Fluor-633 fluorescence was used to visualize dendritic morphology. To avoid photodynamic damage of the cell, the laser intensity was set to 0.1% (of 5 and 25 mW, respectively). This allowed us to obtain stable Ca²⁺ images over at least one hour. To measure the temporal profile of [Ca²⁺] changes at the spine head, we use line scanning mode (500 Hz, FIG. 5E-F). Baseline fluorescence (F₀) was measured for 50 ms prior to the stimulus and ΔF/F was calculated as (ΔF/F)ₜ=(Fₜ−F₀)/F₀. We periodically monitored AP-evoked [Ca²⁺] transients as an index of dendritic viability. Data analysis was performed using ImagePro Plus (Media Cybernetics, Carlsbad, Calif.).

Results

Since neither reducing global activity (through TTX, NBQX, or flunitrazepam) or Ca²⁺ flux (through blockade of just VGCC channels or partial blockade of NMDA receptors) were effective in long term treatments, we wished to examine whether a selective reduction of different aspects of the network's patterns of Ca²⁺ flux would be capable of generating long-term potentiability. In functional neural networks, the synaptic inputs induced by physiological stimulus tend to be correlated, while inputs from spontaneous activity of neural connections are uncorrelated (Lisman, 1997). Since the effects of Mg²⁺ block are strongest at resting potentials, the extracellular concentration of Mg²⁺ ([Mg²⁺]ₒ) influences Ca²⁺ flux during uncorrelated synaptic inputs. By contrast, during depolarization, the magnesium block is removed completely. Thus, an elevation of [Mg²⁺]ₒ, increases the blockade of NMDA channels during hyperpolarization, selectively reducing Ca²⁺ flux associated with uncorrelated activity while leaving depolarization-associated Ca²⁺ flux relatively unperturbed. The results described above, showing that increasing the [Mg²⁺]ₒ resulted in a long term increase in synaptic plasticity, suggested to us that the long term efficacy of Mg⁺⁺ might be due to its ability to cause a selective reduction in Ca⁺⁺ flux associated with uncorrelated activity.

Although the biophysics of Mg²⁺ block have been studied extensively (Mayer et al., 1984; Nowak et al., 1984; Jahr and Stevens, 1990), we wished to determine precisely the behavior of the Mg²⁺ block at near-physiologic concentrations. For example, while the original studies looked at a range from 0.5 to 10 mM of Mg²⁺, this mineral is found in rodent CSF at a concentration of around 0.8 mM (Chutkow, 1974), and in humans, CSF [Mg²⁺]ₒ fluctuates between 1.0 and 1.2 mM (Kapaki et al., 1989). We evoked NMDA currents by iontophoretic application of glutamate to a putative single bouton identified by FM 1-43 labeling and recorded under whole cell patch clamp in the presence of the AMPAR blocker, NBQX (10 μM). The glutamate delivered by this technique can be focal and rapid (Murnick et al., 2002) and the evoked NMDA currents run with a time course roughly comparable to that of EPSC_{NMDA} (Renger et al., 2001). When [Mg²⁺]ₒ was raised from 0.8 to 1.2 mM, it caused a 50% reduction of EPSC_{NMDA} amplitude at −50 mV, while the size of the outward EPSC_{NMDA} at +40 mV remained relatively unchanged. Beyond a concentration of 1.2 mM [Mg²⁺]ₒ, further increases in [Mg 2+] had significantly less effect on current.

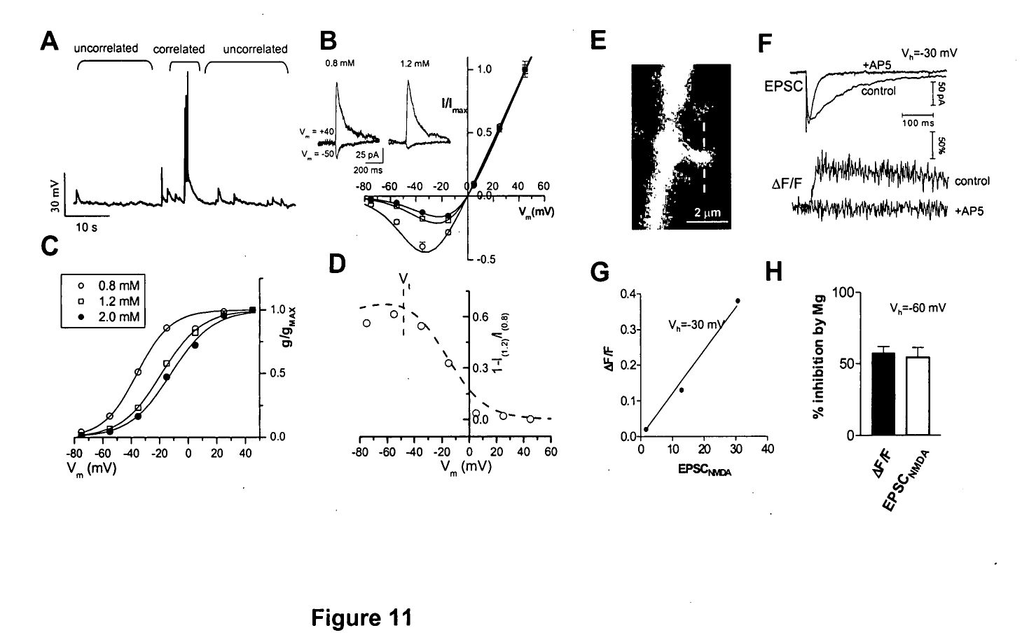

To quantify this effect, we converted our measured NMDA currents into conductance (g). The voltage-dependency of the Mg block did not vary significantly with increases in [Mg²⁺]. However, apparent affinity, K_{Mg}, changed dramatically when [Mg²⁺]ₒ increased from 0.8 to 1.2 mM (FIG. 11C and Table 1). Thus, at the known physiological range of CSF [Mg²⁺]ₒ, small variations in [Mg²⁺]ₒ have profound influences on the affinity of Mg²⁺ for NMDAR. This region of high magnesium sensitivity could be used to selectively change the NMDAR-mediated Ca²⁺ flux near resting membrane potentials. To illustrate this effect quantitatively, we plotted the ratio of NMDA current amplitudes as a function of membrane potential at 0.8 and 1.2 mM [Mg²⁺]ₒ (FIG. 11D). An increase in [Mg²⁺]ₒ from 0.8 to 1.2 mM led to a ˜60% reduction of NMDA current when the membrane potential was below −50 mV, but had no effect at depolarized potentials.

It is known that NMDA channels are highly Ca²⁺ permeable (P_{Ca}/P_{Na}˜10) (Mayer and Westbrook, 1987; Jahr and Stevens, 1993) and Ca²⁺ influx through NMDARs accounts for most of synaptic spine Ca²⁺ (Yuste and Denk, 1995; Kovalchuk et al., 2000; Sabatini et al., 2002). In particular, since an elegant recent study by Sabatini et al (2002) has shown that the time course of Ca²⁺ influx during NMDARs opening matches exactly with the temporal profile of Ca²⁺ lifespan in the spine, measured NMDAR currents can be used to predict the size of the Ca²⁺ flux. However, since our conclusions rest on the effects that perturbation of Ca²⁺ flux has on synaptic plasticity, we wished to directly confirm that changes of Mg²⁺ concentration cause a proportional change in NMDA-mediated [Ca²⁺] transients at single spines. Individuals spines were visualized by filling the cell with both a fluorescent Ca²⁺ indicator dye (Fluo-5F) and a Ca 2+-insensitive Alexa 633 (Sabatini et al., 2002) (FIG. 11E). NMDARs at a single spine were activated by local iontophoresis of glutamate while the resulting [Ca²⁺] transients were being monitored (FIG. 11F). Consistent with the studies mentioned above, we observed that the amplitude of synaptic NMDA currents and that of [Ca²⁺] transients at the spine were linearly correlated (r²=0.99, FIG. 11G). Finally, to test whether Ca²⁺ flux has a similar sensitivity to [Mg²⁺]ₒ as NMDA currents, we compared the [Ca²⁺] transients and the NMDA currents at the same spines before and after an acute increase of [Mg²⁺]ₒ. The amplitude of Ca²⁺ signal (ΔF/F) and the amplitude of EPSC_{NMDA} were reduced by 57±5 and 54±7%, respectively (N=3, p>0.3). Thus, the NMDA-mediated currents and Ca²⁺ flux are sensitive to physiological variations of [Mg²⁺]ₒ.

As a control, we determined that raising [Mg²⁺]ₒ from 0.8 to 1.2 mM did not alter resting membrane potential (74±1 mV at 0.8 mM; 73±1 mV at 1.2 mM, N=5), nor overall synaptic inputs during network activity (EPSC_{1.2}/EPSC_{0.8}=1.03±0.1, N=5). Furthermore, there were no detectable changes in spontaneous miniature EPSP_{AMPA}s (EPSP_{1.2}/EPSP_{0.8}=0.9±0.1, N=5). The lack of detectable influence on the excitability of neurons and synaptic inputs during network activity can be explained by the relatively small increase in [Mg²⁺]ₒ, which is insufficient to alter significantly the excitability of membrane or the probability of transmitter release.

TABLE 1
Woodhull parameters for voltage dependency of Mg²⁺ block as a
function of [Mg²⁺]ₒ.

| [Mg²⁺]ₒ (mM) | K_{Mg} (mM) | δ | n |
|---|---|---|---|
| 0.8 | 16.3 ± 1.0 | 1.00 ± 0.02 | 5 |
| 1.2 | 4.5 ± 0.3 | 0.90 ± 0.03 | 5 |
| 2.0 | 4.5 ± 0.4 | 0.87 ± 0.06 | 5 |

### Example 8

### Plastic Synapses Exhibit Upregulation of NMDAR Function

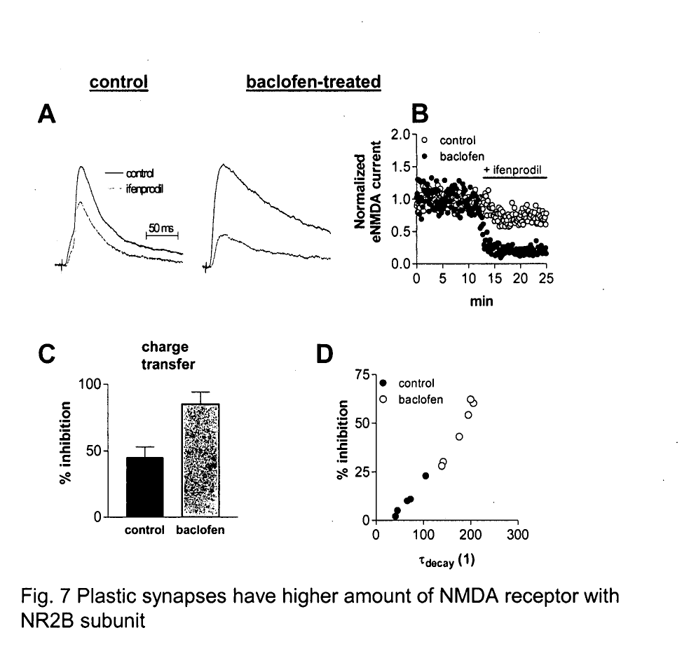

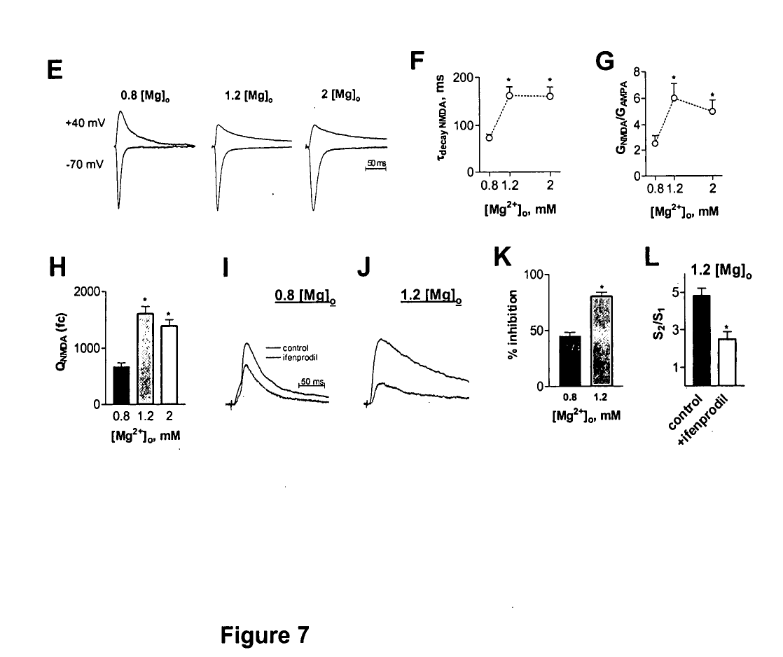

We sought to determine on a molecular basis what mediates the effects of reducing the level of neural activity and/or the background level of Ca++ flux at postsynaptic locations, so as to turn synapses plastic. We hypothesized that one target may be the number and properties of NMDA receptors at individual synapses. Previous studies suggest that down-regulation of synaptic plasticity during postnatal development is correlated with a change of NMDA receptor subunit-composition [Carmignoto & Vicini 1992; Fox & Zahs 1994; Craig & Malenka 1995]. Thus, we tested whether the enhancement of synaptic plasticity is associated with an up-regulation of functional NMDA receptor contribution to synaptic transmission. Evoked NMDA currents (EPSC_{NMDA}) were measured by the double-perforated patch technique in the presence of 1 mM NBQX and picrotoxin (50 mM). FIG. 7A shows EPSC_{NMDA} recorded from the neurons in control cultures and cultures treated with baclofen. It is clear that EPSC_{NMDA} recorded from baclofen treated neurons decays much slower than that from control neurons (FIG. 7A left, black curve, τ_{decay}=50 ms and FIG. 7A right, baclofen-treated, black curve, τ_{decay}=200 ms). On the average, τ_{decay} of EPSC_{NMDA} was 66±25 ms (N=6) in control and 176±30 ms (N=6) in baclofen-treated neurons, showing a significant inverse correlation between EPSC_{NMDA} duration and network activity (p<0.0001).

Previous studies have indicated that NMDA current duration is largely controlled by the subunit composition of NMDA receptors: receptors containing NR2B exhibit longer currents than those associated with NR2A [Flint 1997; Vicini 1998; Tovar 2000]. The prolonged decay of EPSC_{NMDA} in the baclofen-treated neurons might result from an increase in the number of NR2B-containing NMDA receptors or reduction in NR2A containing receptors. To test this possibility, we compared the sensitivity of EPSC_{NMDA} to ifenprodil, a selective NR2B blocker, in control versus treated neurons. FIGS. 7A and 7B show that, indeed, the ifenprodil-sensitivity of baclofen-treated cultures is much higher than in controls. On average, in baclofen-treated cultures, application of 3 μM ifenprodil reduced the amplitude of EPCS_{NMDA} by 77% (±6, N=6, p<0.001), the decay time-constant by 45% (±12, N=6, p<0.001), and the total charge transfer by 85% (±9, N=6, p<0.001, FIG. 7C, gray bars). In contrast, in control cultures, the amplitude was reduced only by 33% (±8, N=6, p<0.001), the decay time-constant by 8% (±5, N=6, p>0.1), and the total charge transfer by 45% (±8, N=6, p<0.01, FIG. 7C). Interestingly, for all neurons tested, the magnitude of ifenprodil block was positively and tightly correlated with the duration of EPCS_{NMDA} before drug application (FIG. 7D), confirming the specificity of ifenprodil in the blockade of NMDA receptors containing NR2B subunit.

We also examined EPCS_{NMDA} under similar conditions in cultures that had been treated with elevated [Mg⁺⁺]ₒ at various concentrations. FIG. 7E shows representative EPSCs from neurons cultured under various [Mg], showing that the decay of EPSC₊₄₀ from elevated Mg²⁺-treated synapses was significantly slower. Further analysis indicated that this is associated with the slower decay of EPSC_{NMDA} (FIG. 7F). To get the relative strength of EPSC_{NMDA} over EPSC_{AMPA}, we calculated an N/A ratio (defined as G_{NMDA}/G_{AMPA}, G being the integrated conductance of each current). FIG. 7G shows that the N/A ratio increased significantly in elevated Mg²⁺ treatment neurons (p=0.02, N=5 for each condition). From this ratio and the charge transfer of quantal AMPAR mediated transmission (266±23 fC, N=6), we calculated the quantal charge transfer though NMDARs (Q_{NMDA}, integrated from t=0 to 500 ms). Q_{NMDA} increased by 2.4 fold in neurons cultured with 1.2 mM [Mg²⁺]ₒ (FIG. 7H). Given the 2-fold change in EPSC_{NMDA} decay, this increase is likely to be entirely due to the prolongation of NMDA currents.

The prolonged decay of EPSC_{NMDA} in the elevated Mg²⁺-treatment neurons might result from an increase in the number of NR2B-containing NMDARs. To test this possibility we determined the sensitivity of EPSC_{NMDA} to ifenprodil in control versus experimental neurons. FIGS. 7I and 7J show that, indeed, the ifenprodil-sensitivity of EPSC_{NMDA} in elevated Mg-treated cultures is much higher than in the controls. On average, in 1.2 mM Mg-treated cultures, an application of 3 μM ifenprodil reduces the total charge transfer by 80% (±4, N=5, p<0.001). In contrast, in control cultures, the total charge transfer was reduced only by 45% (±2, N=6, p<0.01, FIG. 7K).

These results suggest that on the postsynaptic side, a reduction in background Ca²⁺ flux induces a 50% increase in NR2B-containing NMDARs and the N/A ratio. To directly evaluate the role of increased NMDAR function in the enhancement of plasticity of presynaptic terminals, we applied an NR2B-selective antagonist, ifenprodil (1 μM, current reduced by 53% (N=3, p<0.01) to cancel the up-regulation of NMDAR function induced by the elevation of [Mg²⁺]ₒ. If the plasticity we observed was due to changes strictly other than increased NMDAR function, then this perturbation should have had no effect on the enhanced potentiation. However, the magnitude of potentiation was reduced by 50% suggesting an involvement of NMDARs up-regulation in the enhancement of synaptic plasticity (FIG. 7L).

### Example 9

### Mg⁺⁺ Treatment Enhances Cognition and Expression of Memory-Associated Protein In Vivo

Materials and Methods

Determination of [Mg⁺⁺] in CSF. Total Mg⁺⁺ levels in CSF in control and Mg⁺⁺-treated rats were determined by a colorimetric method with xylidyl blue (Anilytics Incorporated, MD) (Thomas, L. (1998). Clinical Laboratory diagnostics. 1^{st} ed. Francfurt: TH-Books Verlagsgesellschaft, 231-41.)

Quantitative Western blotting. Samples of hippocampal homogenate (10 ug/sample) were resolved on polyacrylamide gels, transferred to PVDF, and probed with anti-NR1 (Chemicon), NR2A (Upstate) or NR2B (Santa Cruz Biotech), GluR1 (Chemicon), Synapsin1 (American Qualex), or PSD-95 (Upstate) antibody followed by the appropriate secondary antibody coupled to horseradish peroxidase (1:10,000, Santa Cruz Biotech) in Tris-buffered saline (pH 7.3) containing 5% dry milk and 0.1% Tween (BioRad). Visualization of immunoreactive bands was achieved by enhanced chemiluminescence (Perkin Elmer Biosciences) and captured on autoradiography film (Kodak Scientific). Standard curves, in which the amount of protein was varied systematically, were constructed to establish that we operated within the linear range of the chemiluminescence detection method. In addition, each immunoblot was exposed to autoradiography film multiple times (average 7; minimum 5) for varying duration to make sure we operated in the linear range of the film for each experiment. Digital images, produced by scanning films on a Epson 3200 Scanner (Epson) with Epson Scan Software (Epson), were quantified using GelPro Analyzer 3.1 software. The integrated optical density (IOD) of each immunoreactive band from hippocampal samples relative to baseline measured immediately above and below the band in the same lane, and normalized by the actin or neurofilament band also in the same lane. Group data (eight animals per group) are represented as the mean+/−SEM of the IOD of samples.

Behavior test: Novel object recognition. The subjects were 20 experimentally naive, female, Sprague-Dawley rats (Charles River), 1 year old at the beginning of the experiment. They were housed two per cage with continuous access to food and water under a 12:12 light-dark cycle, with light onset at 8:00 a.m. Rats in the Mg⁺⁺-treated group received drinking water containing 6 mg/kg/day MgCl₂ for 4 months before experiments.

The rats were first habituated to the open-field arena by allowing them to explore it for two daily 15-min sessions. Two identical objects were present in the arenas during habituation sessions; those objects were not used in the subsequent experimental trials. The rat was placed into the arena with two identical sample objects and allowed to explore for 10 min (familiarization phase). For the double-training test, the familiarization phase was repeated with the interval of 10 min. The rat was then removed and the objects were replaced with two new objects; one of the new objects was identical to the sample and the other was a novel object that the rat had never before encountered. The rat was returned to the arena for the retention test and allowed to explore for 5 min (retention test). The retention intervals were 10 min and 24 hours. Rats spent the retention interval in a cage in the colony room. Objects were cleaned thoroughly between trials with 20% ethanol solution to ensure the absence of olfactory cues. The particular objects for a given trial were randomly determined, but each object was used for only one trial per rat.

The criteria for exploration were strictly based on active exploration, in which rats had to be sniffing or touching the object with the nose and/or forepaws. The main dependent measure was the exploration ratio, that is, the proportion of total object-exploration that was spent exploring the novel object (tₙₒᵥₑₗ/[tₙₒᵥₑₗ+t_{familiar}]) during the 5-min test phase. To determine whether the rats discriminated between the novel and familiar objects, the exploration ratios obtained under each condition were compared with what would be expected by chance (i.e., a ratio of ˜50%), using one-sample Student's t tests.

Results

To determine whether the treatments that proved effective in increasing synaptic plasticity in hippocampal neural culture are also effective in vivo, we extended our work to studies in rats. We noted that the extracellular concentration Mg⁺ in the CSF of a normal human brain is about 1.2 mM, the same concentration at which we obtained the maximum synaptic plasticity in vitro (FIG. 8B). We tested whether increasing Mg⁺⁺ concentration might also lead to enhanced synaptic plasticity in rats in vivo. Since Mg⁺⁺ is an essential mineral, normally obtained only by ingestion, we added a low quantity of Mg⁺⁺ (˜6 mg/kg/day) to drinking water in attempt to raise CSF Mg⁺⁺ concentration. This amount was proportional to that recommended by the FDA as a daily food supplement for humans. Two groups of adult Sprague-Dawley rats (8 months) were used as experimental populations. Rats were otherwise maintained under standard laboratory conditions.

First, we tested whether the CSF level of [Mg] could be up-regulated by increasing [Mg] in drinking water. FIG. 8A shows that chronic increase of [Mg] in drinking water results in elevation of the CSF [Mg] level from 1.06±0.03 mM to 1.2±0.05 (P<0.05, N=8 per group). In our in vitro study we demonstrated that an increase of [Mg] in this range has a profound effect on the plasticity of presynaptic terminals, as described above.

Since the increase in extracellular concentration of Mg⁺⁺ in vitro appeared to lead to the enhancement of NMDA receptors containing the NR2B subunit, we checked the level of NR2B expression in the control and treated animals. Additionally, we compared other synaptic proteins that might influence the plasticity of glutamatergic synapses. FIGS. 8C-8F show results of Western blots in 8 control, and 8 Mg⁺⁺-treated 1-year old rats. The amounts of NR2B and GluR1 were increased by approximately 60% in the treated animals, while no significant changes were found for NR2A, NR1, or synapsin1 (actin was used for normalization). We did not observe any significant change in the general status, such as body weight and mobility, of the rats that received water containing Mg⁺⁺. Thus, this strategy, derived from our studies of in vitro preparations, is also effective in intact animals.

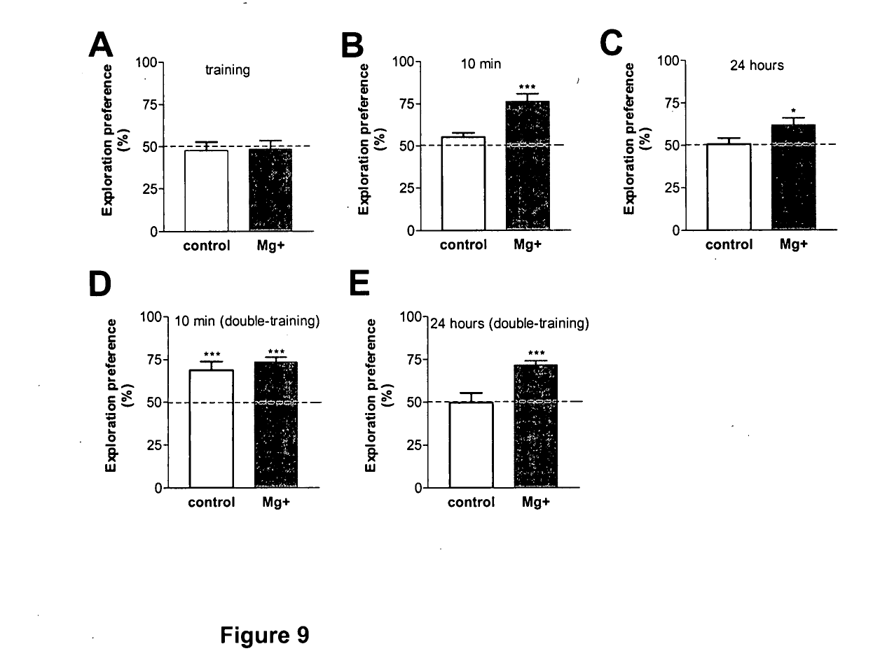

The next step was to test whether an increase in the CSF [Mg] level leads to improvement of learning and memory of 1-year old rats. We tested recognition memory, which is evolutionary conserved in species including rodent, primates and human (rev. Mumby, 2001). In the object recognition task, there was no significant difference among groups in the total time exploring both identical objects during training (23.8±2.2 and 24.1±2.5 sec, for control and Mg-treated groups, respectively, P>0.4) and in the time exploring any of the two identical objects during training (FIG. 9A). This indicates that the Mg-treatment had no effect on exploratory activity or motivation.

Results for recognition memory retention are shown in FIG. 9. Animals in the control group did not show significant preference towards the novel object during both the short- (FIG. 9B) and long-term (FIG. 9C) memory tests, indicating learning/memory impairment in 1-year old rats. Rats without learning/memory impairment would have spent more time exploring the novel object since they would have remembered the object to which they had previously been exposed. However, the Mg-treated group shows a significant preference (75%) to the novel object during short-term memory test. This preference declined to 60% following 24 hours. These results indicate that Mg⁺⁺ treatment improved the ability of aged rats to learn and/or remember the object to which they had been previously exposed.

To further confirm that the difference between groups in the short-term memory task does not result from effect of Mg⁺⁺ on curiosity and motivation, we decreased the difficulty of the test using double-trail training during which animals experienced two familiarization phases. Following this test, both groups showed similar significant preference towards the novel object during the short-term memory test (P<0.001). However, only the Mg-treated group retained the same level of preference for 24 hours, while the exploration preference of the control group was returned to the basal level. These results show that chronic Mg treatment increases the time course of recognition memory decline, i.e., rats treated with Mg⁺⁺ remember the object to which they were previously exposed for longer than untreated rats. Similar results to those obtained for 1 year old rats were also obtained for rats aged 1.5 years.

### Example 10

### GABA_{B} Agonist Treatment Enhances Expression of Memory-Associated Protein In Vivo

Our in vitro results demonstrate that selective decrease of Ca⁺⁺ flux during uncorrelated activity by treatment with Mg⁺⁺, an endogenous voltage-dependent blocker of NMDA channel, results in long-lasting or permanent enhancement of synaptic plasticity. Another way to selectively decrease Ca⁺⁺ flux during uncorrelated activity is to enhance presynaptic inhibition. Since the action of presynaptic modulators appears to be dependent on the activity level of their target synapses, i.e., they inhibit transmitter release at low frequencies of stimulation without affecting (or increasing) transmission at high stimulation frequencies (Markram and Tsodyks, 1996; Abbott et al., 1997; Isaacson and Hille, 1997; Brenowitz et at al., 1998), we recognized that activation of presynaptic receptors by their agonists would decrease selectively transmission during uncorrelated activity resulting in a decrease in background Ca⁺ flux. To test if presynaptic inhibition plays an important role in determining intrinsic plasticity of synapses, we used baclofen, a selective agonist of GABA_{B} receptors to enhance presynaptic inhibition. As described above, we found that short term application of baclofen (10 μM) to a neural network caused increased synaptic plasticity. We repeated the experiment over a longer time period and showed that application of baclofen (10 μM) induced enhancement of plasticity for both short term (4-6 hours) and long term (48 hours) periods of time (FIG. 10A). Moreover, baclofen triggered increased sensitivity of the EPSC_{NMDA} to ifenprodil, a selective blocker of NR2B subunits indicating that there is an increase in NR2B-containing NMDARs (FIG. 10B).

Since treatment with the selective GABA_{B} agonist baclofen resulted in long-lasting enhancement of plasticity in vitro, we next tested the effect of baclofen in vivo. 18-month old rats were treated for 4 months with 0.07 mg/kg/day of baclofen (added to the drinking water) and protein expression analysis was done in control and baclofen-treated groups as described above. Our results demonstrate that this low dose of baclofen triggered significant enhancement of NR2B and GLUR1 expression level (actin was used for normalization) (FIGS. 10C and 10D). We did not observe any significant change in the general status of the rats treated with baclofen. These results suggest that chronic treatment with baclofen induces up-regulation of proteins crucial for synaptic plasticity and learning and memory (Tang et al., 1999).

### Additional Embodiments

The foregoing description is to be understood as being representative only and is not intended to be limiting. Alternative methods for reducing overall neural activity and/or reducing Ca⁺⁺ flux into excitatory neurons and locations are intended to be included within the accompanying claims. In particular, compositions and methods of treatment involving drugs that act on pathways and by mechanisms discussed herein are encompassed even if not explicitly listed. One of ordinary skill in the art will readily be able to identify such known compounds by referring to, e.g., Goodman and Gilman, supra, and/or the scientific literature. It is also noted that although the invention has been described in terms of methods for enhancing synaptic plasticity, it will be evident that in general, synaptic plasticity can be reduced if so desired by modifying certain of the methods in ways that are readily apparent. For example, since exposing a neural network to increased Mg⁺⁺ concentrations enhances synaptic plasticity, it is evident that decreasing the Mg⁺⁺ concentration below that normally experienced will reduce synaptic plasticity. Similarly, where activators or inhibitors of particular receptors or channels are useful for enhancing synaptic plasticity, inhibitors or activators, respectively, of the same receptors or channels will be useful for reducing synaptic plasticity. Reducing synaptic plasticity may be desirable when a subject is expected to experience an event that he or she does not wish to remember, e.g., a painful, embarrassing, or stressful event. By reducing synaptic plasticity prior to the event, the subject may avoid forming memories that will subsequently be unpleasant to recall.

### REFERENCES

ABBOTT, F., VARELA, J A, SEN, K., AND NELSON, S B (1997). Synaptic depression and cortical gain control. Science 275, 220-224.
ALLEN, C., and STEVENS, C. F. (1994). An evaluation of causes for unreliability of synaptic transmission. Proc Natl Acad Sci USA 91, 10380-10383.
ANGEHAGEN M, BEN-MENACHEM E, RONNBACK L, HANSSON E. (2003) Novel mechanisms of action of three antiepileptic drugs, vigabatrin, tiagabine, and topiramate. Neurochem Res. 28(2):333-40
BEAR, M. F. (2003). Bidirectional synaptic plasticity: from theory to reality. Philos Trans R Soc Lond B Biol Sci 358, 649-655.
BEAR, M. F., KLEINSCHMIDT, A., GU, Q. A. & SINGER, W. (1990). Disruption of experience-dependent synaptic modifications in striate cortex by infusion of an NMDA receptor antagonist. J Neurosci 10, 909-925.
BETTLER B, KAUPMANN K, MOSBACHER J, GASSMANN M. (2004) Molecular structure and physiological functions of GABA(B) receptors. Physiol Rev. 84(3):835-67.
BETZ, W. J. & BEWICK, G. S. (1992). Optical analysis of synaptic vesicle recycling at the frog neuromuscular junction. Science 255, 200-203.
BI, G. Q. & POO, M. M. (1998). Synaptic modifications in cultured hippocampal neurons: dependence on spike timing, synaptic strength, and postsynaptic cell type. J Neurosci 18, 10464-10472.
BLISS, T. V., AND COLLINGRIDGE, G. L. (1993). A synaptic model of memory: long-term potentiation in the hippocampus. Nature 361, 31-39.
BOWERY N G, BETTLER B, FROESTL W, GALLAGHER J P, MARSHALL F, RAITERI M, BONNER T I, ENNA S J. (2002) International Union of Pharmacology. XXXIII. Mammalian gamma-aminobutyric acid(B) receptors: structure and function. Pharmacol Rev. 54(2):247-64.
BRENOWITZ, S., DAVID, J., AND TRUSSELL, L. (1998). Enhancement of Synaptic Efficacy by Presynaptic GABA_{B} Receptors. Neuron 20: 135-141.
BURNS, A. & ZAUDIG, M (2002). Mild cognitive impairment in older people. The Lancet 360, 1963-1965.
CARMIGNOTO, G. & VICINI, S. (1992). Activity-dependent decrease in NMDA receptor responses during development of the visual cortex. Science 258, 1007-1011.
CHOI, S., KLINGAUF, J. & TSIEN, R. W. (2000). Postfusional regulation of cleft glutamate concentration during LTP at ‘silent synapses’. Nat Neurosci 3, 330-336.
CHUTKOW, J. G. (1974). Metabolism of magnesium in central nervous system. Relationship between concentrations of magnesium in cerebrospinal fluid and brain in magnesium deficiency. Neurology 24, 780-787.
CLARK, C. M. & KARLAWISH, J. H. T. (2003). Alzheimer disease: current concepts and emerging diagnostic and therapeutic strategies. Ann. Intern. Med. 138: 400-410.
CLINE, H. T. & CONSTANTINE-PATON, M. (1989). NMDA receptor antagonists disrupt the retinotectal topographic map. Neuron 3, 413-426.
COLLINGRIDGE, G. L., KEHL, S. J. & McLENNAN, H. (1983). Excitatory amino acids in synaptic transmission in the Schaffer collateral-commissural pathway of the rat hippocampus. J Physiol 334, 33-46.
COTTRELL, J. R., DUBE, G. R., EGLES, C. & Liu, G. (2000). Distribution, density, and clustering of functional glutamate receptors before and after synaptogenesis in hippocampal neurons. J Neurophysiol 84, 1573-1587.
CRAIR, M. C. & MALENKA, R. C. (1995). A critical period for long-term potentiation at thalamocortical synapses. Nature 375, 325-328.
de Jager, C A, (2004) Changes over time in memory, processing speed and clock drawing tests help to discriminate between vascular cognitive impairment, mild cognitive impairment and Alzheimer's disease. Neurol Res. 26(5):481-7.
DUFFY S, NGUYEN P V, BAKER G B. (2004) Phenylethylidenehydrazine, a novel GABA-transaminase inhibitor, reduces epileptiform activity in rat hippocampal slices. Neuroscience. 126(2):423-32.
FAGIOLINI, M. & HENSCH, T. K. (2000). Inhibitory threshold for critical-period activation in primary visual cortex. Nature 404, 183-186.
FAGIOLINI, M., KATAGIRI, H., MIYAMOTO, H., MORI, H., GRANT, S. G., MISHINA, M. & HENSCH, T. K. (2003). Separable features of visual cortical plasticity revealed by N-methyl-D-aspartate receptor 2A signaling. Proc Natl Acad Sci USA 100, 2854-2859.
FAGIOLINI, M., PIZZORUSSO, T., BERARDI, N., DOMENICI, L. & MAFFEI, L. (1994). Functional postnatal development of the rat primary visual cortex and the role of visual experience: dark rearing and monocular deprivation. Vision Res 34, 709-720.
FELDMAN, D. E. (2001). A new critical period for sensory map plasticity. Neuron 31, 171-173.
FERGUSON, S. S. G. (2001). Evolving concepts in G protein-coupled receptor endocytosis: the role in receptor desensitization and signaling. Pharm. Rev. 53:1-24.
FLINT, A. C., MAISCH, U. S., WEISHAUPT, J. H., KRIEGSTEIN, A. R. & MONYER, H. (1997). NR2A subunit expression shortens NMDA receptor synaptic currents in developing neocortex. J Neurosci 17, 2469-2476.
Fox, K. & ZAHS, K. (1994). Critical period control in sensory cortex. Curr Opin Neurobiol 4, 112-119.
GARDONI, F., CAPUTI, A., CIMINO, M., PASTORINO, L., CATTABENI, F. & DI LUCA, M. (1998). Calcium/calmodulin-dependent protein kinase II is associated with NR2A/B subunits of NMDA receptor in postsynaptic densities. J Neurochem 71, 1733-1741.
GORDON, J. A. & STRYKER, M. P. (1996). Experience-dependent plasticity of binocular responses in the primary visual cortex of the mouse. J Neurosci 16, 3274-3286.
GU, Q. A., BEAR, M. F. & SINGER, W. (1989). Blockade of NMDA-receptors prevents ocularity changes in kitten visual cortex after reversed monocular deprivation. Brain Res Dev Brain Res 47, 281-288.
HENSCH, T. K., FAGIOLINI, M., MATAGA, N., STRYKER, M. P., BAEKKESKOV, S. & KASH, S. F. (1998). Local GABA circuit control of experience-dependent plasticity in developing visual cortex. Science 282, 1504-1508.
HOUX, P., et al., (2002) Testing cognitive function in elderly populations: the PROSPER study, Journal of Neurology Neurosurgery and Psychiatry 73:385-389.
HUANG, Z. J., KIRKWOOD, A., PIZZORUSSO, T., PORCIATTI, V., MORALES, B., BEAR, M. F., MAFFEI, L. & TONEGAWA, S. (1999). BDNF regulates the maturation of inhibition and the critical period of plasticity in mouse visual cortex. Cell 98, 739-755.
HUBEL, D. H. & WIESEL, T. N. (1970). The period of susceptibility to the physiological effects of unilateral eye closure in kittens. J Physiol 206, 419-436.
ISAACSON, J S AND HILLE, B. (1996). GABA_{B}-mediated presynaptic inhibition of excitatory transmission and synaptic vesicle dynamics in cultured hippocampal neurons. Neuron 18, 143-152.
IWASATO, T., DATWANI, A., WOLF, A. M., NISHIYAMA, H., TAGUCHI, Y., TONEGAWA, S., KNOPFEL, T., ERZURUMLU, R. S. & ITOHARA, S. (2000). Cortex-restricted disruption of NMDAR1 impairs neuronal patterns in the barrel cortex. Nature 406, 726-731.
JAHR, C. E. & STEVENS, C. F. (1990). A quantitative description of NMDA receptor-channel kinetic behavior. J Neurosci 10, 1830-1837.
JONES, M. V. & WESTBROOK, G. L. (1996). The impact of receptor desensitization on fast synaptic transmission. Trends in Neurosci. 19(3), 96-101.
JONES K A, BOROWSKY B, TAMM J A, CRAIG D A, DURKIN M M, DAI M, YAO W J, JOHNSON M, GUNWALDSEN C, HUANG L Y, TANG C, SHEN Q, SALON J A, MORSE K, LAZ T, SMITH K E, NAGARATHNAM D, NOBLE S A, BRANCHEK T A, GERALD C. (1998) GABA(B) receptors function as a heteromeric assembly of the subunits GABA(B)R1 and GABA(B)R2 Nature 396(6712):674-9.
KAPAKI, E., Segditsa, J., and Papageorgiou, C. (1989). Zinc, copper and magnesium concentration in serum and CSF of patients with neurological disorders. Acta Neurol Scand 79, 373-378.
KARLAWISH, J. H. T. & CLARK, C. M. (2003). Diagnostic evaluation of elderly patients with mild memory problems. Ann. Intern. Med. 138: 411-419.
KATZ, L. C. & SHATZ, C. J. (1996). Synaptic activity and the construction of cortical circuits. Science 274, 1133-1138.
KAUPMANN K, HUGGEL K, HEID J, FLOR P J, BISCHOFF S, MICKEL S J, MCMASTER G, ANGST C, BITTIGER H, FROESTL W, BETTLER B. (1997) Expression cloning of GABA(B) receptors uncovers similarity to metabotropic glutamate receptors, Nature. 20; 386(6622):239-46.
KAUPMANN K, MALITSCHEK B, SCHULER V, HEID J, FROESTL W, BECK P, MOSBACHER J, BISCHOFF S, KULIK A, SHIGEMOTO R, KARSCHIN A, BETTLER B. (1998) GABA(B)-receptor subtypes assemble into functional heteromeric complexes. Nature. 396(6712):683-7.
KERR D I, ONG J, PUSPAWATI N M, PRAGER R H. (2002) Arylalkylamines are a novel class of positive allosteric modulators at GABA(B) receptors in rat neocortex. Eur J. Pharmacol. 451(1):69-77.
KERR D I, ONG J. (2003) Potentiation of metabotropic GABA(B) receptors by L-amino acids and dipeptides in rat neocortex. Eur J Pharmacol. 468(2): 103-108.
KOVALCHUK, Y., Eilers, J., Lisman, J., and Konnerth, A. (2000). NMDA receptor-mediated subthreshold Ca(2+) signals in spines of hippocampal neurons. J Neurosci 20, 1791-1799.
KUTSUWADA, T., SAKIMURA, K., MANABE, T., TAKAYAMA, C., KATAKURA, N., KUSHIYA, E., NATSUME, R., WATANABE, M., INOUE, Y., YAGI, T., AIZAWA, S., ARAKAWA, M., TAKAHASHI, T., NAKAMURA, Y., MORI, H. & MISHINA, M. (1996). Impairment of suckling response, trigeminal neuronal pattern formation, and hippocampal LTD in NMDA receptor epsilon 2 subunit mutant mice. Neuron 16, 333-344.
LEONARD, A. S., LIM, I. A., HEMSWORTH, D. E., HORNE, M. C. & HELL, J. W. (1999). Calcium/calmodulin-dependent protein kinase II is associated with the N-methyl-D-aspartate receptor. Proc Natl Acad Sci USA 96, 3239-3244.
LI, Y., ERZURUMLU, R. S., CHEN, C., JHAVERI, S. & TONEGAWA, S. (1994). Whisker-related neuronal patterns fail to develop in the trigeminal brainstem nuclei of NMDAR1 knockout mice. Cell 76, 427-437.
LISMAN, J. E. (1997). Bursts as a unit of neural information: making unreliable synapses reliable. Trends Neurosci 20, 38-43.
LIU, G. (2003). Presynaptic control of quantal size: kinetic mechanisms and implications for synaptic transmission and plasticity. Curr Opin Neurobiol 13, 324-331.
LIU, G. & TSIEN, R. W. (1995). Properties of synaptic transmission at single hippocampal synaptic boutons. Nature 375, 404-408.
LU, H. C., GONZALEZ, E. & CRAIR, M. C. (2001). Barrel cortex critical period plasticity is independent of changes in NMDA receptor subunit composition. Neuron 32, 619-634.
MAEJIMA, T., HASHIMOTO, K., YOSHIDA, T., AIBA, A. & KANO, M. (2001). Presynaptic inhibition caused by retrograde signal from metabotropic glutamate to cannabinoid receptors. Neuron 31(3): 463-75.
MALINOW, R. & MALENKA, R. C. (2002). AMPA receptor trafficking and synaptic plasticity. Annu Rev Neurosci 25, 103-126.
MARKRAM, H. AND M. TSODYKS, M. (1996). Redistribution of synaptic efficacy between neocortical pyramidal neurons. Nature 382, 807-810.
MARTIN, S. J., GRIMWOOD, P. D. & MORRIS, R. G. M. (2000). Synaptic Plasticity and Memory: An Evaluation of the Hypothesis., Annu. Rev. Neurosci., 23: 649-711.
MARAGOS, W. F., CHU, D. C., YOUNG, A. B., D'AMATO, C. J. & PENNEY, J. B., JR. (1987). Loss of hippocampal [3H]TCP binding in Alzheimer's disease. Neurosci Lett 74, 371-376.
MAYER, M. L., WESTBROOK, G. L. & GUTHRIE, P. B. (1984). Voltage-dependent block by Mg2+ of NMDA responses in spinal cord neurones. Nature 309, 261-263.
McHUGH, T. J., BLUM, K. I., TSIEN, J. Z., TONEGAWA, S. & WILSON, M. A. (1996). Impaired hippocampal representation of space in CA1-specific NMDAR1 knockout mice. Cell 87, 1339-1349.
MEHTA, M. R., QUIRK, M. C. & WILSON, M. A. (2000). Experience-dependent asymmetric shape of hippocampal receptive fields. Neuron 25, 707-715.
MONTGOMERY, J. M., PAVLIDIS, P. & MADISON, D. V. (2001). Pair recordings reveal all-silent synaptic connections and the postsynaptic expression of long-term potentiation. Neuron 29, 691-701.
MUMBY, D. G. (2001). Perspectives on object-recognition memory following hippocampal damage: lessons from studies in rats. Behav. Brain Research, 127: 159-181
MURNICK, J. G., DUBE, G., KRUPA, B. & LIU, G. (2002). High-resolution iontophoresis for single-synapse stimulation. J Neurosci Methods 116, 65-75.
MURTHY, V. N., SCHIKORSKI, T., STEVENS, C. F. & ZHU, Y. (2001). Inactivity produces increases in neurotransmitter release and synapse size. Neuron 32, 673-682.
MURTHY, V. N., SEJNOWSKI, T. J. & STEVENS, C. F. (1997). Heterogeneous release properties of visualized individual hippocampal synapses. Neuron 18, 599-612.
NG G Y, CLARK J, COULOMBE N, ETHIER N, HEBERT T E, SULLIVAN R, KARGMAN S, CHATEAUNEUF A, TSUKAMOTO N, MCDONALD T, WHITING P, MEZEY E, JOHNSON M P, LIU Q, KOLAKOWSKI L F JR, EVANS J F, BONNER T I, O'NEILL G P. (1999) Identification of a GABAB receptor subunit, gb2, required for functional GABAB receptor activity J Biol. Chem. 274(12):7607-10.
NOWAK, L., BREGESTOVSKI, P., ASCHER, P., HERBET, A. & PROCHIANTZ, A. (1984). Magnesium gates glutamate-activated channels in mouse central neurones. Nature 307, 462-465.
O'BRIEN, J. A., LEMAIRE, W., CHEN, T. B., CHANG, R. S., JACOBSON, M. A., HA, S. N., LINDSLEY, C. W., SCHAFFHAUSER, J. H., SUR, C., PETTIBONE, D. J., CONN, P. J. & WILLIAMS, D. L. (2003). A family of highly selective allosteric modulators of the metabotropic glutamate receptor subtype 5. Mol. Pharmacol. 64(3): 731-740.
PAN Y, QIU J, SILVERMAN R B. (2003) Design, synthesis, and biological activity of a difluoro-substituted, conformationally rigid vigabatrin analogue as a potent gamma-aminobutyric acid aminotransferase inhibitor. J Med. Chem. 46(25):5292-3.
PETERSEN, R. C., DOODY, R., KURZ, A., MOHS, R. C., MORRIS, J. C., RABINS, P. V., RITCHIE, K., ROSSOR, M., THAL, L. & WINBLAD, B. (2001). Current Concepts in Mild Cognitive Impairment. Arch. Neurol. 58, 1985-1992.
PHILPOT, B. D., WEISBERG, M. P., RAMOS, M. S., SAWTELL, N. B., TANG, Y. P., TSIEN, J. Z. & BEAR, M. F. (2001). Effect of transgenic overexpression of NR2B on NMDA receptor function and synaptic plasticity in visual cortex. Neuropharmacology 41, 762-770.
QUINLAN, E. M., OLSTEIN, D. H. & BEAR, M. F. (1999a). Bidirectional, experience-dependent regulation of N-methyl-D-aspartate receptor subunit composition in the rat visual cortex during postnatal development. Proc Natl Acad Sci USA 96, 12876-12880.
QUINLAN, E. M., PHILPOT, B. D., HUGANIR, R. L. & BEAR, M. F. (1999b). Rapid, experience-dependent expression of synaptic NMDA receptors in visual cortex in vivo. Nat Neurosci 2, 352-357.
REISBERG, B., DOODY, R. STOFFLER, A., SCMITT, A., FERIS, S., MOBIUS, H. J., MEMANTINE STUDY GROUP. (2003) Memantine in moderate-to-severe Alzheimer's disease. N Engl J Med. 348(14): 1333-41
RENGER, J. J., EGLES, C. & LIU, G. (2001). A developmental switch in neurotransmitter flux enhances synaptic efficacy by affecting AMPA receptor activation. Neuron 29, 469-484.
RYAN, T. A., REUTER, H., WENDLAND, B., SCHWEIZER, F. E., TSIEN, R. W. & SMITH, S. J. (1993). The kinetics of synaptic vesicle recycling measured at single presynaptic boutons. Neuron 11, 713-724.
RYAN, T. A., SMITH, S. J. & REUTER, H. (1996). The timing of synaptic vesicle endocytosis. Proc Natl Acad Sci USA 93, 5567-5571.
SABATINI, B. L., OERTNER, T. G. & SVOBODA, K. (2002). The life cycle of Ca(2+) ions in dendritic spines. Neuron 33, 439-452.
SANES, J. R. & LICHTMAN, J. W. (1999). Development of the vertebrate neuromuscular junction. Annu Rev Neurosci 22, 389-442.
SAVICC, N., LULTHI, A., GAAHWILER, B. H. & MCKINNEY, R. A. (2003). N-methyl-D-aspartate receptor blockade during development lowers long-term potentiation threshold without affecting dynamic range of CA3-CA1 synapses. Proc Natl Acad Sci USA 100, 5503-5508.
SCHOUSBOE A, SARUP A, LARSSON O M, WHITE H S. (2004) GABA transporters as drug targets for modulation of GABAergic activity Biochem Pharmacol. 15; 68(8): 1557-63.
SELKOE, D. (2002). Alzheimer's disease is a synaptic failure. Science 298(5594):789-91.
SHATZ, C. J. & STRYKER, M. P. (1978). Ocular dominance in layer IV of the cat's visual cortex and the effects of monocular deprivation. J Physiol 281, 267-283.
SHENG, M., CUMMINGS, J., ROLDAN, L. A., JAN, Y. N. & JAN, L. Y. (1994). Changing subunit composition of heteromeric NMDA receptors during development of rat cortex. Nature 368, 144-147.
SHI, J., AAMODT, S. M. & CONSTANTINE-PATON, M. (1997). Temporal correlations between functional and molecular changes in NMDA receptors and GABA neurotransmission in the superior colliculus. J Neurosci 17, 6264-6276.
SZE, C., BI, H., KLEINSCHMIDT-DEMASTERS, B. K., FILLEY, C. M. & MARTIN, L. J. (2001). N-Methyl-D-aspartate receptor subunit proteins and their phosphorylation status are altered selectively in Alzheimer's disease. J Neurol Sci 182, 151-159.
TANG, Y. P., SHIMIZU, E., DUBE, G. R., RAMPON, C., KERCHNER, G. A., ZHUO, M., Liu, G. & TSIEN, J. Z. (1999a). Genetic enhancement of learning and memory in mice. Nature 401, 63-69.
TANG, Y. P., SHIMIZU, E., DUBE, G. R., RAMPON, C., KERCHNER, G. A., ZHUO, M., LIU, G. & TSIEN, J. Z. (1999b). Genetic enhancement of learning and memory in mice [see comments]. Nature 401, 63-69.
THOMAS, L. (1998). Clinical Laboratory diagnostics. 1^{st} ed. Frankfurt: TH-Books Verlagsgesellschaft, 231-41.
THIAGARAJAN, T. C., Piedras-Renteria, E. S., and Tsien, R. W. (2002). alpha- and betaCaMKII. Inverse regulation by neuronal activity and opposing effects on synaptic strength. Neuron 36, 1103-1114.
TOMINAGA, T., TOMINAGA, Y., YAMADA, H., MATSUMOTO, G. & ICHIKAWA, M. (2000). Quantification of optical signals with electrophysiological signals in neural activities of Di-4-ANEPPS stained rat hippocampal slices. J Neurosci Methods 102, 11-23.
TOVAR, K. R., SPROUFFSKE, K. & WESTBROOK, G. L. (2000). Fast NMDA receptor-mediated synaptic currents in neurons from mice lacking the epsilon2 (NR2B) subunit. J Neurophysiol 83, 616-620.
TURRIGIANO, G. G., LESLIE, K. R., DESAI, N. S., RUTHERFORD, L. C. & NELSON, S. B. (1998). Activity-dependent scaling of quantal amplitude in neocortical neurons. Nature 391, 892-896.
URWYLER S, MOSBACHER J, LINGENHOEHL K, HEID J, HOFSTETTER K, FROESTL W, BETTLER B, KAUPMANN K. (2001) Positive allosteric modulation of native and recombinant gamma-aminobutyric acid(B) receptors by 2,6-Di-tert-butyl-4-(3-hydroxy-2,2-dimethyl-propyl)-phenol (CGP7930) and its aldehyde analog CGP13501. Mol Pharmacol. 60(5):963-71.
URWYLER S, POZZA M F, LINGENHOEHL K, MOSBACHER J, LAMPERT C, FROESTL W, KOLLER M, KAUPMANN K. (2003) N,N′-Dicyclopentyl-2-methylsulfanyl-5-nitro-pyrimidine-4,6-diamine (GS39783) and structurally related compounds: novel allosteric enhancers of gamma-aminobutyric acidB receptor function. J Pharmacol Exp Ther. 307(1):322-30.
U.S. Xyrem Multicenter Study Group (2004). Sodium oxybate demonstrates long-term efficacy for the treatment of cataplexy in patients with narcolepsy. Sleep Med. 5(2):119-23.
VACHER C M, BETTLER B. (2003) GABA(B) receptors as potential therapeutic targets. Curr Drug Targets CNS Neurol Disord. 2(4):248-59
VICINI, S., WANG, J. F., LI, J. H., ZHU, W. J., WANG, Y. H., LUO, J. H., WOLFE, B. B. & GRAYSON, D. R. (1998). Functional and pharmacological differences between recombinant N-methyl-D-aspartate receptors. J Neurophysiol 79, 555-566.
WOODHULL, A. M. (1973). Ionic blockage of sodium channels in nerve. J Gen Physiol 61, 687-708.
WU, M., NEWTON, S. S., ATKINS, J. B., XU, C., DUMAN, R. S. & ALREJA, M. (2003). Acetylcholinesterase inhibitors activate septohippocampal GABAergic neurons via muscarinic but not nicotinic receptors. J. Pharmacol. Exp. Ther., Sep 9 [Epub ahead of print].
YUSTE, R., and DENK, W. (1995). Dendritic spines as basic functional units of neuronal integration. Nature 375, 682-684.
ZAKHARENKO, S. S., ZABLOW, L. & SIEGELBAUM, S. A. (2001). Visualization of changes in presynaptic function during long-term synaptic plasticity. Nat Neurosci 4, 711-717.

We claim:

1. A method of enhancing cognitive function in a subject comprising steps of:
(i) identifying a subject in need of enhancement of cognitive function; and
(ii) administering to the subject a composition comprising a compound that selectively decreases Ca⁺⁺ influx associated with uncorrelated activity into excitatory neurons.

2. The method of claim 1, wherein the composition comprises a GABA_{B} receptor activator.

3. The method of claim 2, wherein the composition comprises a GABA_{B} receptor agonist.

4. The method of claim 3, wherein the GABA_{B} receptor agonist is selected from the group consisting of: 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, 3-amino-2-(4-chlorophenyl)-1-nitropropane, (3-aminopropyl)phosphonous acid, (4-aminobut-2-yl)phosphonous acid, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, β-amino-2-(4-methylphenyl)propyl]phosphonous acid, β-amino-2-(4-trifluoromethylphenyl)propyl]phosphonous acid, β-amino-2-(4-methoxyphenyl)propyl]phosphonous acid, β-amino-2-(4-chlorophenyl)-2-hydroxypropyl]phosphonous acid, (3-amino propyl)methylphosphinic acid, (3-amino-2-hydroxypropyl)methylphosphinic acid, (3aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methyl phosphinic acid, (3-aminopropyl)hydroxymethylphosphinic acid, (5-aminopent-3-yl)methylphosphinic acid, (4-amino-1,1,1-trifluorobut-2-yl)methylphosphinic acid, and (3-amino-2-(4-chlorophenyl)propyl)sulfinic acid, and 3-aminopropylsulfinic acid.

5. The method of claim 3, wherein the GABA_{B} receptor agonist is selected from the group consisting of: 4-amino-3-(4-chlorophenyl)butanoic acid (baclofen), (3-aminopropyl)methylphosphinic acid, (3-amino-2-hydroxypropyl)methylphosphinic acid, 4-aminobutanoic acid (GABA), (3-amino-2-(4-chlorophenyl)propyl)sulfinic acid, (3-aminopropyl)(difluoromethyl)phosphinic acid, (3-amino-2-oxo-propyl)methyl phosphinic acid, 4-amino-3-(5-chlorothien-2-yl)butanoic acid, and (3-aminopropyl)phosphonous acid.

6. The method of claim 3, wherein the GABA_{B} receptor agonist is selected from the group consisting of: CGP27492, CGP35024), CGP44532, CGP44533, or CGP34938.

7. The method of claim 3, wherein the GABA_{B} receptor agonist is baclofen.

8. The method of claim 7, wherein the baclofen is administered at a total daily dose of 10 mg or less.

9. The method of claim 7, wherein the baclofen is administered at a total daily dose of 5 mg or less.

10. The method of claim 3, wherein the GABA_{B} receptor agonist is administered at a total daily dose that is equipotent to that of 10 mg or less of baclofen.

11. The method of claim 3, wherein the GABA_{B} receptor agonist is administered at a total daily dose that is equipotent to that of 5 mg or less of baclofen.

12. The method of claim 2, wherein the GABA_{B} receptor activator is provided in the form of a magnesium salt.

13. The method of claim 2, wherein the GABA_{B} receptor activator is selective for GABA_{B} receptors.

14. The method of claim 2, wherein the GABA_{B} receptor activator is substantially inactive at GABA_{A} and GABA_{C} receptors.

15. The method of claim 2, wherein the GABA_{B} receptor activator is administered for a period of at least two weeks.

16. The method of claim 2, wherein the composition comprises a positive GABA_{B} receptor modulator.

17. The method of claim 16, wherein the positive GABA_{B} receptor modulator is a positive allosteric modulator.

18. The method of claim 16, wherein the positive GABA_{B} receptor modulator is selected from the group consisting of: CGP7930 [2,6-Di-tert-butyl-4-(3-hydroxy-2,2-dimethyl-propyl)-phenol], CGP13501, N,N′-Dicyclopentyl-2-methylsulfanyl-5-nitro-pyrimidine-4,6-diamine (GS39783), and arylalkyl amines.

19. The method of claim 2, wherein the composition further comprises an acetylcholinesterase inhibitor.

20. The method of claim 2, wherein the composition further comprises magnesium.

21. The method of claim 1, wherein the compound that selectively decreases Ca⁺⁺ influx into excitatory neurons that is associated with uncorrelated activity imposes a voltage-dependent block on NMDA receptors.

22. The method of claim 21, wherein the voltage-dependent block is readily reversible.

23. The method of claim 21, wherein the compound is able to impose a voltage-dependent block on NMDA receptors under physiological conditions.

24. The method of claim 21, wherein the compound is able to impose a voltage-dependent block on NMDA receptors under pathological conditions.

25. The method of claim 21, wherein the compound is a divalent cation.

26. The method of claim 25, wherein the divalent cation is Mg⁺⁺ or Zn⁺⁺.

27. The method of claim 25, wherein the divalent cation is Mg⁺⁺.

28. The method of claim 27, wherein the Mg⁺⁺ is provided in an aqueous solution.

29. The method of claim 27, wherein the Mg⁺⁺ is provided in a readily bioavailable form.

30. The method of claim 27, wherein the composition comprises MgCl₂, magnesium lactate, magnesium citrate, magnesium aspartate, magnesium glycinate, magnesium chelazome®, and MgSO₄.

31. The method of claim 27, wherein the composition is administered in an amount sufficient to raise the magnesium concentration in the subject's CSF by between approximately 0.05 mM and approximately 0.5 mM.

32. The method of claim 27, wherein the composition is administered in an amount sufficient to raise the magnesium concentration in the subject's CSF by between approximately 0.05 mM and approximately 0.3 mM.

33. The method of claim 27, wherein the composition is administered in an amount sufficient to raise the magnesium concentration in the subject's CSF by between approximately 0.05 mM and approximately 0.2 mM.

34. The method of claim 27, wherein between 1-20 mg/kg/day Mg is administered.

35. The method of claim 27, wherein between 5-10 mg/kg/day Mg is administered.

36. The method of claim 27, wherein 800 mg/day Mg or less is administered.

37. The method of claim 27, wherein between 100 and 200 mg/day Mg is administered.

38. The method of claim 27, wherein between 200 and 400 mg/day Mg is administered.

39. The method of claim 27, wherein between 400 and 800 mg/day Mg is administered.

40. The method of claim 25, wherein the composition further comprises a GABA_{B} receptor activator.

41. The method of claim 40, wherein the GABA_{B} receptor activator is baclofen.

42. The method of claim 25, wherein the composition further comprises an acetylcholinesterase inhibitor.

43. The method of claim 21, wherein the composition is administered for at least two weeks.

44. The method of claim 1, wherein the subject is at risk of or suffering from a conditions selected from the group consisting of: memory impairment, dementia, cognitive deficit, or attention deficit disorder.

45. The method of claim 1, wherein the subject is at risk of or suffering from a disease or condition selected from the group consisting of: Alzheimer's disease, age-associated memory impairment, or mild cognitive impairment.

46. The method of claim 1, wherein the composition is administered in an amount and for a time sufficient to treat or prevent memory impairment.

47. The method of claim 1, wherein the composition is administered orally.

48. The method of claim 1, wherein the composition is administered for at least two weeks.

49. A method of enhancing cognitive function in a subject comprising steps of:
(i) identifying a subject in need of enhancement of cognitive function; and
(ii) administering to the subject a composition comprising a GABA_{B} receptor activator.

50. The method of claim 49, wherein the composition comprises a GABA_{B} receptor agonist.

51. The method of claim 50, wherein the GABA_{B} receptor agonist is selected from the group consisting of: 4-aminobutanoic acid (GABA), 4-amino-3-(4-chlorophenyl)butanoic acid (baclofen), 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, 3-amino-2-(4-chlorophenyl)-1-nitropropane, (3-aminopropyl)phosphonous acid, (4-aminobut-2-yl)phosphonous acid, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl)propyl]phosphonous acid, [3-amino-2-(4-methoxyphenyl)propyl]phosphonous acid, [3-amino-2-(4-chlorophenyl)-2-hydroxypropyl]phosphonous acid, (3-amino propyl)methylphosphinic acid, (3-amino-2-hydroxypropyl)methylphosphinic acid, (3-aminopropyl)(difluoromethyl)phosphinic acid, (4-aminobut-2-yl)methylphosphinic acid, (3-amino-1-hydroxypropyl)methylphosphinic acid, (3-amino-2-hydroxypropyl)(difluoromethyl)phosphinic acid, (E)-(3-aminopropen-1-yl)methylphosphinic acid, (3-amino-2-oxo-propyl)methyl phosphinic acid, (3-aminopropyl)hydroxymethylphosphinic acid, (5-aminopent-3-yl)methylphosphinic acid, (4-amino-1,1,1-trifluorobut-2-yl)methylphosphinic acid, and (3-amino-2-(4-chlorophenyl)propyl)sulfinic acid, and 3-aminopropylsulfinic acid.

52. The method of claim 50, wherein the GABA_{B} receptor agonist is selected from the group consisting of: 4-amino-3-(4-chlorophenyl)butanoic acid (baclofen), (3aminopropyl)methylphosphinic acid, (3-amino-2-hydroxypropyl)methylphosphinic acid, 4-aminobutanoic acid (GABA), (3-amino-2-(4-chlorophenyl)propyl)sulfinic acid, (3-aminopropyl)(difluoromethyl)phosphinic acid, (3-amino-2-oxo-propyl)methyl phosphinic acid, 4-amino-3-(5-chlorothien-2-yl)butanoic acid, and (3-aminopropyl)phosphonous acid.

53. The method of claim 50, wherein the GABA_{B} receptor agonist is selected from the group consisting of: CGP27492, CGP35024), CGP44532, CGP44533, or CGP34938.

54. The method of claim 50, wherein the GABA_{B} receptor agonist is baclofen.

55. The method of claim 54 wherein the baclofen is administered at a total daily dose of 10 mg or less.

56. The method of claim 54, wherein the baclofen is administered at a total daily dose of 5 mg or less.

57. The method of claim 50, wherein the GABA_{B} receptor agonist is administered at a total daily dose that is equipotent to that of 10 mg or less of baclofen.

58. The method of claim 50, wherein the GABA_{B} receptor agonist is administered at a total daily dose that is equipotent to that of 5 mg or less of baclofen.

59. The method of claim 49, wherein the GABA_{B} receptor activator is provided in the form of a magnesium salt.

60. The method of claim 49, wherein the GABA_{B} receptor activator is selective for GABA_{B} receptors.

61. The method of claim 49, wherein the GABA_{B} receptor activator is substantially inactive at GABA_{A} and GABA_{C} receptors.

62. The method of claim 49, wherein the GABA_{B} receptor activator is administered for a period of at least two weeks.

63. The method of claim 49, wherein the composition comprises a positive GABA_{B} receptor modulator.

64. The method of claim 63, wherein the positive GABA_{B} receptor modulator is a positive allosteric modulator.

65. The method of claim 63, wherein the positive GABA_{B} receptor modulator is selected from the group consisting of: CGP7930 [2,6-Di-tert-butyl-4-(3-hydroxy-2,2-dimethyl-propyl)-phenol], CGP 13501, N,N′-Dicyclopentyl-2-methylsulfanyl-5-nitro-pyrimidine-4,6-diamine (GS39783), and arylalkyl amines.

66. The method of claim 49, wherein the composition further comprises an acetylcholinesterase inhibitor.

67. The method of claim 49, wherein the composition further comprises magnesium.

68. The method of claim 49, wherein the subject is at risk of or suffering from a conditions selected from the group consisting of: memory impairment, dementia, cognitive deficit, or attention deficit disorder.

69. The method of claim 49, wherein the subject is at risk of or suffering from a disease or condition selected from the group consisting of: Alzheimer's disease, age-associated memory impairment, or mild cognitive impairment.

70. The method of claim 49, wherein the composition is administered in an amount and for a time sufficient to treat or prevent memory impairment.

71. The method of claim 49, wherein the composition is administered orally.

72. The method of claim 49, wherein the composition is administered for at least two weeks.

73. A composition comprising a solid dosage form containing 5 mg or less of baclofen.

74. The composition of claim 73, further comprising an acetylcholinesterase inhibitor.

75. A composition comprising a solid dosage form containing an amount of a GABA_{B} agonist that is equipotent to 5 mg or less of baclofen.

76. The composition of claim 75, further comprising an acetylcholinesterase inhibitor.

77. A method of enhancing cognitive function in a subject comprising steps of:
(i) identifying a subject in need of enhancement of cognitive function; and
(ii) administering to the subject a composition comprising a compound that imposes a voltage-dependent block on NMDA receptors.

78. The method of claim 77, wherein the voltage-dependent block is readily reversible.

79. The method of claim 77, wherein the compound is able to impose a voltage-dependent block on NMDA receptors under physiological conditions.

80. The method of claim 77, wherein the compound is able to impose a voltage-dependent block on NMDA receptors under pathological conditions.

81. The method of claim 77, wherein the compound is a divalent cation.

82. The method of claim 81, wherein the divalent cation is Mg⁺⁺ or Zn⁺⁺.

83. The method of claim 81, wherein the divalent cation is Mg⁺⁺.

84. The method of claim 83, wherein the Mg⁺⁺ is provided in an aqueous solution.

85. The method of claim 83, wherein the Mg⁺⁺ is provided in a readily bioavailable form.

86. The method of claim 83, wherein the composition comprises MgCl₂, magnesium lactate, magnesium citrate, magnesium aspartate, magnesium glycinate, magnesium chelazome®, and MgSO₄.

87. The method of claim 83, wherein the composition is administered in an amount sufficient to raise the magnesium concentration in the subject's CSF by between approximately 0.05 mM and approximately 0.5 mM.

88. The method of claim 83, wherein the composition is administered in an amount sufficient to raise the magnesium concentration in the subject's CSF by between approximately 0.05 mM and approximately 0.3 mM.

89. The method of claim 83, wherein the composition is administered in an amount sufficient to raise the magnesium concentration in the subject's CSF by between approximately 0.05 mM and approximately 0.2 mM.

90. The method of claim 83, wherein between 1-20 mg/kg/day Mg is administered.

91. The method of claim 83, wherein between 5-10 mg/kg/day Mg is administered.

92. The method of claim 83, wherein 800 mg/day Mg or less is administered.

93. The method of claim 83, wherein between 100 and 200 mg/day Mg is administered.

94. The method of claim 83, wherein between 200 and 400 mg/day Mg is administered.

95. The method of claim 83, wherein between 400 and 800 mg/day Mg is administered.

96. The method of claim 81, wherein the composition further comprises a GABA_{B} receptor activator.

97. The method of claim 96, wherein the GABA_{B} receptor activator is baclofen.

98. The method of claim 77, wherein the composition further comprises an acetylcholinesterase inhibitor.

99. The method of claim 77, wherein the composition is administered for at least two weeks.

100. The method of claim 77, wherein the subject is at risk of or suffering from a conditions selected from the group consisting of: memory impairment, dementia, cognitive deficit, or attention deficit disorder.

101. The method of claim 77, wherein the subject is at risk of or suffering from a disease or condition selected from the group consisting of: Alzheimer's disease, age-associated memory impairment, or mild cognitive impairment.

102. The method of claim 77, wherein the composition is administered in an amount and for a time sufficient to treat or prevent memory impairment.

103. The method of claim 77, wherein the composition is administered orally.

104. The method of claim 77, wherein the composition is administered for at least two weeks.

105. A vessel containing a fluid having magnesium dissolved therein, wherein the vessel is labeled to indicate that it contains magnesium and that its contents are of use for enhancement of cognitive function and/or memory, or for the treatment or prevention of Alzheimer's disease or forgetfulness.

106. A container containing a solid dosage form comprising magnesium, wherein the container is labeled to indicate that it contains magnesium and that its contents are of use for enhancement of cognitive function and/or memory, or for the treatment or prevention of Alzheimer's disease or forgetfulness.

107. A method for enhancing synaptic plasticity in a neural network comprising the steps of:
providing a neural network in which it is desired to enhance synaptic plasticity; and
exposing the neural network to a composition comprising a compound that reduces overall Ca⁺⁺ flux into excitatory synapses in the neural network.

108. The method of claim 107, wherein the compound selectively decreases Ca⁺⁺ influx associated with uncorrelated activity into excitatory neurons in the neural network.

109. The method of claim 107, wherein the compound reduces Ca⁺⁺ flux into postsynaptic terminals of excitatory synapses in the neural network.

110. The method of claim 107, wherein the composition does not reduce overall neural activity of excitatory neurons in the neural network.

111. The method of claim 107, wherein the composition comprises a compound that imposes a voltage-dependent block on NMDA receptors.

112. The method of claim 111, wherein the block is readily reversible.

113. The method of claim 107, wherein the composition comprises a compound of a class selected from the group consisting of: divalent cations, NMDA receptor inhibitors, AMPA receptor inhibitors, mGluR1 and/or mGluR5 activators, GABA_{B} receptor activators, GABA_{A} receptor activators, muscarinic ACh receptor activators including AChE inhibitors, A1 adenosine receptor activators, voltage-gated Ca⁺⁺ channel inhibitors, and voltage-gated Na⁺ channel inhibitors.

114. The method of claim 113, wherein the composition comprises compounds from at least two of the compound classes.

115. The method of claim 113, wherein the composition comprises compounds from at least three of the compound classes.

116. The method of claim 113, wherein the composition comprises compounds from at least four of the compound classes.

117. The method of claim 113, wherein the composition comprises a divalent cation, which may be present as a salt.

118. The method of claim 113, wherein the composition does not comprise both an NMDA receptor inhibitor other than Mg⁺⁺ and an ACHE inhibitor.

119. The method of claim 113, wherein the composition does not comprise both an NMDA receptor inhibitor and an ACHE inhibitor.

120. The method of claim 113, wherein if the composition comprises both an NMDA receptor inhibitor and an AChE inhibitor then it comprises an additional compound selected from a class other than the NMDA receptor inhibitor class or the ACHE inhibitor class.

121. The method of claim 113, wherein the composition comprises magnesium or a magnesium salt comprising Mg⁺⁺.

122. The method of claim 113, wherein the composition comprises an NMDA receptor inhibitor.

123. The method of claim 113, wherin the composition comprises a compound that blocks NMDA receptors in a voltage-dependent manner.

124. The method of claim 113, wherein the composition comprises an AMPA receptor inhibitor.

125. The method of claim 113, wherein the composition comprises an mGluR1 and/or mGluR5 activator.

126. The method of claim 113, wherein the composition comprises a GABA_{A} receptor activator.

127. The method of claim 113, wherein the composition comprises a GABA_{B} receptor activator.

128. The method of claim 113, wherein the composition comprises a muscarinic ACh receptor activator.

129. The method of claim 113, wherein the composition comprises an ACHE inhibitor.

130. The method of claim 113, wherein the composition comprises an A1 adenosine receptor activator.

131. The method of claim 113, wherein the composition comprises a GABA_{A} receptor activator.

132. The method of claim 113, wherein the composition comprises a GABA_{B} receptor activator.

133. The method of claim 113, wherein the composition comprises a voltage-gated Ca++channel inhibitor.

134. The method of claim 113, wherein the composition comprises a voltage-gated Na⁺ channel inhibitor.

135. The method of claim 113, wherein the composition comprises at least one compound approved by the U.S. Food and Drug Administration for treatment or prevention of a disease or condition, and wherein the neural network is exposed to a dose of the compound that is subtherapeutic for the disease or condition for which the compound is approved.

136. The method of claim 113, wherein the neural network is exposed to a dose of the compound that does not induce desensitization.

137. The method of claim 107, wherein the composition inhibits release of Ca⁺⁺ from the endoplasmic reticulum.

138. The method of claim 137, wherein the composition comprises a compound that inhibits IP₃ receptors.

139. The method of claim 137, wherein the composition comprises a compound that inhibits ryanodine receptors.

140. The method of claim 107, wherein the composition reduces neural activity of excitatory neurons in the neural network by stimulating the activity of inhibitory neurons that synapse on the excitatory neurons.

141. The method of claim 107, wherein the neural network is a cultured neural network.

142. The method of claim 107, wherein the neural network is present within the central nervous system of a mammalian subject.

143. The method of claim 107, wherein the neural network comprises hippocampal neurons.

144. The method of claim 107, wherein average probability of neurotransmitter release is increased following exposure to the compound.

145. The method of claim 107, wherein average amount of neurotransmitter released per active presynaptic terminal is increased following exposure to the compound.

146. The method of claim 107, wherein both average probability of neurotransmitter release and average amount of neurotransmitter released per active presynaptic terminal are increased following exposure to the compound.

147. The method of claim 107, wherein synaptic strength is increased by approximately two-fold following exposure to the compound.

148. The method of claim 107, wherein synaptic strength is increased by between two-fold and five-fold following exposure to the compound.

149. The method of claim 107, wherein synaptic strength is increased by approximately five-fold following exposure to the compound.

150. The method of claim 107, wherein the composition is administered for between 2 and 12 hours.

151. The method of claim 107, wherein the composition is administered for more than 12 hours.

152. The method of claim 107, wherein the composition is administered for at least 2 weeks.

153. The method of claim 107, wherein the composition is administered for months or years.

154. The method of claim 107, further comprising the step of:
measuring the synaptic plasticity of the neural network.

155. The method of claim 154, wherein the step of measuring comprises measuring (i) presynaptic strength.

156. The method of claim 154, wherein the step of measuring comprises measuring (i) postsynaptic strength.

157. The method of claim 154, wherein the step of measuring comprises measuring both presynaptic strength and postsynaptic strength.

158. A composition comprising at least two compounds, wherein the compounds are members of compound classes selected the group consisting of: divalent cations, NMDA receptor inhibitors, AMPA receptor inhibitors, mGluR1 and/or mGluR5 activators, GABA_{B} receptor activators, GABA_{A} receptor activators, muscarinic ACh receptor activators including ACHE inhibitors, A1 adenosine receptor activators, Ca⁺⁺ channel inhibitors, and Na⁺ channel inhibitors, and wherein at least two of the compounds are members of different compound classes.

159. The composition of claim 158, wherein the composition comprises at least three compounds.

160. The composition of claim 158, wherein the composition comprises compounds from at least three different classes.

161. The composition of claim 158, wherein the composition comprises at least four compounds.

162. The composition of claim 158, wherein the composition comprises compounds from at least four different classes.

163. The composition of claim 158, wherein the composition comprises at least five compounds.

164. The composition of claim 158, wherein the composition comprises compounds from at least five different classes.

165. The composition of claim 158, wherein the composition comprises a divalent cation.

166. The composition of claim 158, wherein the composition comprises magnesium or a salt thereof comprising Mg⁺⁺.

167. The composition of claim 158, wherein the composition comprises an NMDA receptor inhibitor.

168. The composition of claim 158, wherein the composition comprises an AMPA receptor inhibitor.

169. The composition of claim 158, wherein the composition comprises an mGluR1 and/or mGluR5 activator.

170. The composition of claim 158, wherein the composition comprises a GABA_{A} receptor activator.

171. The composition of claim 158, wherein the composition comprises a GABA_{B} receptor activator.

172. The composition of claim 158, wherein the composition comprises a muscarinic ACh receptor activator.

173. The composition of claim 158, wherein the composition comprises an ACHE inhibitor.

174. The composition of claim 158, wherein the composition comprises a Ca⁺⁺ channel inhibitor.

175. The composition of claim 158, wherein the composition comprises an A1 adenosine activator.

176. The composition of claim 158, wherein the composition comprises an Na⁺ channel inhibitor.

177. The composition of claim 158, wherein the composition comprises a divalent cation and a GABA_{A} receptor activator.

178. The composition of claim 158, wherein the composition comprises a divalent cation and a GABA_{B} receptor activator.

179. The composition of claim 158, wherein the composition comprises a divalent cation and a Ca⁺⁺ channel inhibitor.

180. The composition of claim 158, wherein the composition comprises a divalent cation and an ACHE inhibitor.

181. The composition of claim 158, wherein the composition comprises an NMDA receptor inhibitor and an ACHE inhibitor and a compound from a third compound class.

182. The composition of claim 158, wherein the compound classes further include compounds that inhibit release of Ca⁺⁺ from the endoplasmic reticulum.

183. The composition of claim 182, wherein the compound classes that inhibit release of Ca⁺⁺ from the endoplasmic reticulum include IP₃ receptor inhibitors.

184. The composition of claim 182, wherein the compound classes that inhibit release of Ca⁺⁺ from the endoplasmic reticulum include ryanodine receptor inhibitors.

185. The composition of claim 158, wherein if the composition comprises an NMDA receptor inhibitor then it does not comprise an AChE inhibitor.

186. The composition of claim 158, wherein if the composition comprises an NMDA receptor inhibitor and an ACHE inhibitor then it further comprises a third compound from a different compound class.

187. A method of treating or preventing memory impairment comprising steps of:
providing a subject suffering from or at risk of memory impairment; and administering the composition of claim 158, 160, 166, or 185 to the subject.

188. The method of claim 187, wherein the subject is at risk of or suffers from Alzheimer's disease.

189. The method of claim 187, wherein the subject is at risk of or suffers from age-associated memory impairment.

190. The method of claim 187, wherein the subject is at risk of or suffers from mild cognitive impairment.

191. A method of screening a compound comprising steps of:
(i) exposing neurons in a cultured neural network to a detectable substance, wherein the substance is taken up by presynaptic terminals that release neurotransmitter;
(ii) exposing neurons in the neural network to the compound;
(iii) administering a pattern of stimulus to the neurons in the network;
(iv) measuring synaptic plasticity; and
(v) identifying the substance as an enhancer of synaptic plasticity, cognitive function, or both, if the measured synaptic plasticity increases following exposure to the compound.

192. The method of claim 191, wherein the step of measuring synaptic plasticity comprises detecting presynaptic terminals that have taken up the detectable substance and comparing the synaptic strength before and after a stimulus.

193. The method of claim 191, wherein the neurons comprise hippocampal neurons.

194. The method of claim 191, wherein the neurons comprise cortical neurons.

195. The method of claim 191, wherein the detectable substance is a fluorescent dye.

196. The method of claim 192, wherein the stimulus is a theta-burst stimulus.

197. The method of claim 192, wherein the step of measuring comprises measuring (i) average probability of release, (ii) average amount of substance released, or both (i) and (ii).

198. The method of claim 191, wherein the step of measuring comprises acquiring an image of the neural network.
